SEQUENCE LISTING (146 sequences)

SEQ ID NO: 1 - DNA, 43 bp, Artificial
  PCR Primer
aataatggatccgtatccggatttaaaaggaaaagtcgtcgct

SEQ ID NO: 2 - DNA, 42 bp, Artificial
  PCR Primer
aataataagcttaccgcggcctgcctggaatgaaggatattg

SEQ ID NO: 3 - DNA, 42 bp, Artificial
  PCR Primer
aataatcatatgcttgtgaatgtactgatcgtccacgctcac

SEQ ID NO: 4 - DNA, 42 bp, Artificial
  PCR Primer
aataatctcgagtcacgtcagcgggtacaacgcttcatcgaa

SEQ ID NO: 5 - DNA, 31 bp, Artificial
  Linker [GGGSGGGS] direct, HindIII/NdeI
agcttggcggcggcagcggcggcggcagcca

SEQ ID NO: 6 - DNA, 29 bp, Artificial
  Linker [GGGSGGGS] complement, HindIII/NdeI
tatggctgccgccgccgctgccgccgcca

SEQ ID NO: 7 - DNA, 6824 bp, Artificial
  plasmid, pETDuet-busGDH::ppuDp
ggggaattgtgagcggataacaattcccctctagaaataattttgtttaactttaagaaggagatataccatgggcagcagccatcaccatcatcaccacagccaggatccgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 8 - DNA, 42 bp, Artificial
  PCR primer
aataatggatcctatccggatttaaaaggaaaagtcgtcgct

SEQ ID NO: 9 - DNA, 5146 bp, Artificial
  plasmid, pCDFDuet-busGDH-ppuDp
ggggaattgtgagcggataacaattcccctgtagaaataattttgtttaactttaataaggagatataccatggcgccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaattaatacgactcactata

SEQ ID NO: 10 - DNA, 1560 bp, Artificial
  fusion protein, His-busGDH::ppuDp
atgggcagcagccatcaccatcatcaccacagccaggatccgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttggcggcggcagcggcggcggcagccatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacg

SEQ ID NO: 11 - protein, 520 aa, Artificial
  fusion protein, His-busGDH::ppuDp
MGSSHHHHHHSQDPYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRGKLGGGSGGGSHMLVNVLIVHAHPEPQSFTAALRDQAVETFRAQGHQVQVSDLYAMGWNPVASADDFTQRENPEYLVYALEQRQGVKRGAIAADIQQELDKLLWADLLVLNFPIFWFSAPAMLKGWIDRVLVSGVCYGGKRFYDQGGLAGKRALVTVTLGGREHMFGDGAIHGPLEDMLRPILRGTLAYVGFEVLPPFVAWHVPYISADARQDFLQQYRQRLEQLSDDQPLVFPRLAQFDEALYPLT

SEQ ID NO: 12 - DNA, 42 bp, Artificial
  PCR Primer
aataatcatatgtatccggatttaaaaggaaaagtcgtcgct

SEQ ID NO: 13 - DNA, 42 bp, Artificial
  PCR Primer
aataatctcgagaccgcggcctgcctggaatgaaggatattg

SEQ ID NO: 14 - DNA, 39 bp, Artificial
  PCR Primer
aataatctcgagcgtcagcgggtacaacgcttcatcgaa

SEQ ID NO: 15 - DNA, 43 bp, Artificial
  PCR Primer
aataatggatccgaacattttaataacagcttcttcaagagga

SEQ ID NO: 16 - DNA, 42 bp, Artificial
  PCR Primer
aataataagcttaagaagaacgctcctagtcattgctccatc

SEQ ID NO: 17 - DNA, 42 bp, Artificial
  PCR Primer
aataatcatatgaagatagtagttataggatctggaactgct

SEQ ID NO: 18 - DNA, 42 bp, Artificial
  PCR Primer
aataatctcgagtcatgacttaaattttctcatggccatttc

SEQ ID NO: 19 - DNA, 7309 bp, Artificial
  plasmid, pETDuet-pfuGDH::phoDp
taatacgactcactataggggaattgtgagcggataacaattcccctctagaaataattttgtttaactttaagaaggagatataccatgggcagcagccatcaccatcatcaccacagccaggatccgaacattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttggcggcggcagcggcggcggcagccatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 20 - DNA, 50 bp, Artificial
  PCR Primer
aataatccatggcgattttaataacagcttcttcaagaggaataggcttc

SEQ ID NO: 21 - DNA, 5755 bp, Artificial
  plasmid, pCDFDuet-pfuGDH-phoDp
taatacgactcactataggggaattgtgagcggataacaattcccctgtagaaataattttgtttaactttaataaggagatataccatggcgattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 22 - DNA, 2169 bp, Artificial
  fusion protein, His-pfuGDH::phoDp
atgggcagcagccatcaccatcatcaccacagccaggatccgaacattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttggcggcggcagcggcggcggcagccatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtca

SEQ ID NO: 23 - protein, 723 aa, Artificial
  fusion protein, His-pfuGDH::phoDp
MGSSHHHHHHSQDPNILITASSRGIGFNVAKELLKKGHRVVISSSNEKNLEKALEELKEYEKVYPIKADLSKKEEIKNMVEKAWEILGKIDVLVWNAPNVKCEPCMIHELDYEDWVEGALLHIIAPGYLTTLLLNKWLKNNTRGIIIYLSSASVLEPMPPLLLADSTRAGLIQLAKGISRAYGKRGIRAYTVLLGSFDTPGARENLRRLAEKRGVDPEELWKKEVIERTPLQRTGKWEELGELIEFLLSDNAEYMLGSTILFDGAMTRSVLLKLGGGSGGGSHMKIVVIGSGTAGSNFALFMRKLDRKAEIIVIGKEPTMQYSPCALPHVISGTIEKPEDVIVFPNEFYEKQRIKMMLGVEAKKIDRERKVIITDKGEVPYDKLVLATGSRAFIPPIKGVENEGVFTLKSLDDVRRIKEYISKRKPKKVVVIGAGLIGLEGAEAFAKIGMDVLVVELLEHLLPTMLDKDMAKIVQEEMEKHGVKFKFGVGVSEIIGNPVKEVKIGEESVEADLVLVATGVRANVDLAKDAGLEVSRGIVVNEYLQTSDPDIYAIGDCAEVFDAVTGKRTLSQLGTSAVRMAKVAAENIAGKKVKFRPVFNTAITELFDLEIGTFGITEERAKKEGIEVVIGKFKGSTKPEYYPGGKPITVKLIFRKEDKRLIGAQIVGGERVWGRIMTLSALAQKGATVEDVAYLETSYAPPISPTIDPITVAAEMAMRKFKS

SEQ ID NO: 24 - DNA, 42 bp, Artificial
  PCR Primer
aataatcatatgaacattttaataacagcttcttcaagagga

SEQ ID NO: 25 - DNA, 42 bp, Artificial
  PCR Primer
aataatctcgagaagaagaacgctcctagtcattgctccatc

SEQ ID NO: 26 - DNA, 39 bp, Artificial
  PCR Primer
aataatctcgagtgacttaaattttctcatggccatttc

SEQ ID NO: 27 - DNA, 43 bp, Artificial
  PCR Primer
aataatggatccgccttcgcaagtcattcctgaaaaacaaaag

SEQ ID NO: 28 - DNA, 42 bp, Artificial
  PCR Primer
aataataagctttttagaagtctcaacaacatatctaccaac

SEQ ID NO: 29 - DNA, 37 bp, Artificial
  Linker [SGGSGSGGSG] direct, HindIII/NdeI
agcttagcggcggcagcggcagcggcggcagcggcca

SEQ ID NO: 30 - DNA, 35 bp, Artificial
  Linker [SGGSGSGGSG] complement, HindIII/NdeI
tatggccgctgccgccgctgccgctgccgccgcta

SEQ ID NO: 31 - DNA, 6976 bp, Artificial
  plasmid, pETDuet-sceADH::ppuDp
taatacgactcactataggggaattgtgagcggataacaattcccctctagaaataattttgtttaactttaagaaggagatataccatgggcagcagccatcaccatcatcaccacagccaggatccgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttagcggcggcagcggcagcggcggcagcggccatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 32 - DNA, 44 bp, Artificial
  PCR Primer
aataatccatggcgtcgcaagtcattcctgaaaaacaaaaggct

SEQ ID NO: 33 - DNA, 5416 bp, Artificial
  plasmid, pCDFDuet-sceADH-ppuDp
taatacgactcactataggggaattgtgagcggataacaattcccctgtagaaataattttgtttaactttaataaggagatataccatggcgtcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 34 - DNA, 1836 bp, Artificial
  fusion protein, His-sceADH::ppuDp
atgggcagcagccatcaccatcatcaccacagccaggatccgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttagcggcggcagcggcagcggcggcagcggccatatgcttgtgaatgtactgatcgtccacgctcaccccgaaccgcaatcgttcacggcagccctgcgtgaccaggctgtggaaaccttccgtgcccaaggtcatcaggtgcaggtcagtgacttgtacgccatgggctggaacccggtggccagtgccgatgactttacccagcgcgagaaccccgagtacctcgtgtatgcgctggagcagcgtcagggcgtcaagcgcggtgccattgccgcagatatccagcaggagctggacaagctgttgtgggccgatctgctggtgctgaacttcccgatcttctggttctcggccccggccatgctcaagggctggatcgaccgggtgctggtgtcgggggtgtgctatggcggcaagcgcttctacgaccagggcgggttggccggcaagagggcgctggtgaccgtgaccctgggcgggcgcgagcacatgtttggcgacggcgcgattcacgggccgctggaagacatgctgcggccgattctgcggggcacgctggcctatgtcgggttcgaggtgctgccgccgttcgtggcgtggcatgtgccgtacatcagtgctgatgcgcggcaggactttctgcagcaataccggcagcgtctggagcagctctcggacgaccagccactggtgttcccgcgcctggcgcagttcgatgaagcgttgtacccgctgacg

SEQ ID NO: 35 - protein, 612 aa, Artificial
  fusion protein, His-sceADH::ppuDp
MGSSHHHHHHSQDPPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSKKLSGGSGSGGSGHMLVNVLIVHAHPEPQSFTAALRDQAVETFRAQGHQVQVSDLYAMGWNPVASADDFTQRENPEYLVYALEQRQGVKRGAIAADIQQELDKLLWADLLVLNFPIFWFSAPAMLKGWIDRVLVSGVCYGGKRFYDQGGLAGKRALVTVTLGGREHMFGDGAIHGPLEDMLRPILRGTLAYVGFEVLPPFVAWHVPYISADARQDFLQQYRQRLEQLSDDQPLVFPRLAQFDEALYPLT

SEQ ID NO: 36 - DNA, 42 bp, Artificial
  PCR Primer
aataatcatatgccttcgcaagtcattcctgaaaaacaaaag

SEQ ID NO: 37 - DNA, 42 bp, Artificial
  PCR Primer
aataatctcgagtttagaagtctcaacaacatatctaccaac

SEQ ID NO: 38 - DNA, 43 bp, Artificial
  PCR Primer
aataatggatccgtttttcctaaagactaagattatcgaggga

SEQ ID NO: 39 - DNA, 42 bp, Artificial
  PCR Primer
aataataagcttatctccatagaaggccctctcataaattct

SEQ ID NO: 40 - DNA, 7663 bp, Artificial
  plasmid, pETDuet-pfuADH::phoDp
taatacgactcactataggggaattgtgagcggataacaattcccctctagaaataattttgtttaactttaagaaggagatataccatgggcagcagccatcaccatcatcaccacagccaggatccgtttttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaagctggatgaaatggtagagagagcctatgaagatcccctcgttaacttcaacccagttgagccgagaaaagaggacattagaagaatttatgagagggccttctatggagataagcttagcggcggcagcggcagcggcggcagcggccatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 41 - DNA, 44 bp, Artificial
  PCR Primer
aataatccatggcgttcctaaagactaagattatcgagggaagg

SEQ ID NO: 42 - DNA, 6103 bp, Artificial
  plasmid, pCDFDuet-pfuADH-phoDp
taatacgactcactataggggaattgtgagcggataacaattcccctgtagaaataattttgtttaactttaataaggagatataccatggcgttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaagctggatgaaatggtagagagagcctatgaagatcccctcgttaacttcaacccagttgagccgagaaaagaggacattagaagaatttatgagagggccttctatggagataagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtcatgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 43 - DNA, 2523 bp, Artificial
  fusion protein, His-pfuADH::phoDp
atgggcagcagccatcaccatcatcaccacagccaggatccgtttttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaagctggatgaaatggtagagagagcctatgaagatcccctcgttaacttcaacccagttgagccgagaaaagaggacattagaagaatttatgagagggccttctatggagataagcttagcggcggcagcggcagcggcggcagcggccatatgaagatagtagttataggatctggaactgctggaagcaattttgctctcttcatgaggaagctcgataggaaggccgagataatagttataggaaaggaacctacaatgcagtactccccctgtgcccttccacacgttattagcggaaccatagagaagcctgaagacgttatagtgtttccgaatgagttctatgagaagcagagaataaagatgatgcttggagtcgaagctaaaaagatagatagggagagaaaggttataattaccgataagggggaggttccctacgataagctggttcttgctactggatcaagggcttttattcccccgataaagggtgtcgaaaacgagggagtgttcacgctaaagagcctcgatgacgtaaggaggataaaggaatacataagcaagaggaaacctaaaaaggttgtcgttattggcgcaggtttaataggtctagaaggagcagaagcctttgcaaagattggaatggatgttctagttgttgaattactggagcatctattaccgactatgctcgacaaagacatggccaagatcgttcaagaggagatggagaaacatggagttaagttcaagtttggagttggagtcagcgagataataggaaatcctgtcaaggaagttaaaattggagaagagagtgtagaagcagatctagtcctagtggcaacgggagttagggctaatgttgacctagcgaaagatgctgggctggaagtgagtagggggatagttgttaacgagtatcttcagaccagtgatcctgatatatatgctataggagactgtgcagaggtattcgatgccgtaactggaaagagaaccctaagccaattgggaacttctgctgtcagaatggccaaagttgccgctgagaacatagctggaaagaaggtgaaatttaggccagtcttcaacacagctataactgaattattcgacctggaaataggaacatttggaataacagaagagagggctaaaaaggagggaattgaagtcgtaattggaaagtttaaaggttcaactaagccagagtattatcctggaggaaaaccaattacggttaagttaatatttagaaaggaagataagagactaataggggcccaaatagttggaggagagagagtttgggggaggataatgactttatcggcactcgcccagaaaggagctaccgttgaagatgttgcctatctagaaacgagttatgcaccacccataagcccaacgatagacccaatcacggtagccgctgaaatggccatgagaaaatttaagtca

SEQ ID NO: 44 - protein, 841 aa, Artificial
  fusion protein, His-pfuADH::phoDp
MGSSHHHHHHSQDPFFLKTKIIEGRGSLEYLQKEAIGFERALILASSSMKRLGFLKEAEDYLREGGVEVISIAGLPPEPTKDVVDEFLPKVREFSPEILIAIGGGSVIDMAKALKVFYDVPGISFEEVAIKSRFSKPRPVPKLKTPLIAIPSTSGAGSEVSAASVIKVGDVKYTIVTPEIAPEVAILDPRLPEGMPPEVARNSGMDVLVHAIEAYVSKASSPFTDAMAVKAAKAVLEHLEESIRGDKNAREKIHYAATMAGIAFLNARLGLVHAMSHKAAWIGPHGLVNAILLPYVMEYNMARRREKYDALARELGFDNAEELLEEIKALNERLGIGPISSYVSEEEFLAKLDEMVERAYEDPLVNFNPVEPRKEDIRRIYERAFYGDKLSGGSGSGGSGHMKIVVIGSGTAGSNFALFMRKLDRKAEIIVIGKEPTMQYSPCALPHVISGTIEKPEDVIVFPNEFYEKQRIKMMLGVEAKKIDRERKVIITDKGEVPYDKLVLATGSRAFIPPIKGVENEGVFTLKSLDDVRRIKEYISKRKPKKVVVIGAGLIGLEGAEAFAKIGMDVLVVELLEHLLPTMLDKDMAKIVQEEMEKHGVKFKFGVGVSEIIGNPVKEVKIGEESVEADLVLVATGVRANVDLAKDAGLEVSRGIVVNEYLQTSDPDIYAIGDCAEVFDAVTGKRTLSQLGTSAVRMAKVAAENIAGKKVKFRPVFNTAITELFDLEIGTFGITEERAKKEGIEVVIGKFKGSTKPEYYPGGKPITVKLIFRKEDKRLIGAQIVGGERVWGRIMTLSALAQKGATVEDVAYLETSYAPPISPTIDPITVAAEMAMRKFKS

SEQ ID NO: 45 - DNA, 42 bp, Artificial
  PCR Primer
aataatcatatgtttttcctaaagactaagattatcgaggga

SEQ ID NO: 46 - DNA, 42 bp, Artificial
  PCR Primer
aataatctcgagatctccatagaaggccctctcataaattct

SEQ ID NO: 47 - DNA, 43 bp, Artificial
  PCR Primer
aataatggatccgtcaagcatgccaaatcatcaaaaagttgtg

SEQ ID NO: 48 - DNA, 42 bp, Artificial
  PCR Primer
aataataagctttttattttctaattcagctaaaccgtcgtt

SEQ ID NO: 49 - DNA, 42 bp, Artificial
  PCR Primer
aataatcatatgtgcgatacgttcgatctgattgttgttggt

SEQ ID NO: 50 - DNA, 42 bp, Artificial
  PCR Primer
aataatctcgagtcagatatgcagcgggccgtcgaaggccgc

SEQ ID NO: 51 - DNA, 47 bp, Artificial
  Linker [EGKSSGSGSESKST], direct, HindIII/NdeI
agcttgaaggcaaaagcagcggcagcggcagcgaaagcaaaagcacc

SEQ ID NO: 52 - DNA, 46 bp, Artificial
  Linker [EGKSSGSGSESKST], complement, HindIII/ NdeI
tatgggtgcttttgctttcgctgccgctgccgctgcttttgccttc

SEQ ID NO: 53 - DNA, 7594 bp, Artificial
  plasmid, pETDuet-lplLDH::goxDp
taatacgactcactataggggaattgtgagcggataacaattcccctctagaaataattttgtttaactttaagaaggagatataccatgggcagcagccatcaccatcatcaccacagccaggatccgtcaagcatgccaaatcatcaaaaagttgtgttagtcggcgacggcgctgttggttctagttacgcttttgcgatggcacaacaaggaattgctgaagaatttgtaattgtcgatgttgttaaagatcggacaaagggtgacgcccttgatcttgaagacgcccaagcattcaccgctcccaagaagatttactcaggcgaatattcagattgtaaggacgctgacttagttgttattacagccggtgcgcctcaaaagcctggtgaatcacgtttagacttagttaacaagaatttaaatatcctatcatccattgtcaaaccagttgttgactccggctttgacggcatcttcttagttgctgctaaccctgttgacatcttaacttacgctacttggaaattctcaggtttcccaaaggatcgtgtcattggttcagggacttccttagactcttcacgtttacgcgttgcgttaggcaaacaattcaatgttgatcctcgttccgttgatgcttacatcatgggtgaacacggtgattctgaatttgctgcttactcaactgcaaccatcgggacacgtccagttcgcgatgtcgctaaggaacaaggcgtttctgacgaagatttagccaagttagaagacggtgttcgtaacaaagcgtacgacatcatcaacttgaagggtgccacgttctacggtatcgggactgctttaatgcggatttccaaagccattttacgtgatgaaaatgccgttttaccagtaggtgcctacatggacggccaatacggcttaaacgacatttatatcgggactccggctgtgattggtggaactggtttgaaacaaatcatcgaatcaccactttcagctgacgaactcaagaagatgcaagattccgccgcaactttgaaaaaagtgcttaacgacggtttagctgaattagaaaataaaaagcttgaaggcaaaagcagcggcagcggcagcgaaagcaaaagcacccatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 54 - DNA, 44 bp, Artificial
  PCR Primer
aataatccatggcgagcatgccaaatcatcaaaaagttgtgtta

SEQ ID NO: 55 - DNA, 6022 bp, Artificial
  plasmid, pCDFDuet-lplLDH-goxDp
taatacgactcactataggggaattgtgagcggataacaattcccctgtagaaataattttgtttaactttaataaggagatataccatggcgagcatgccaaatcatcaaaaagttgtgttagtcggcgacggcgctgttggttctagttacgcttttgcgatggcacaacaaggaattgctgaagaatttgtaattgtcgatgttgttaaagatcggacaaagggtgacgcccttgatcttgaagacgcccaagcattcaccgctcccaagaagatttactcaggcgaatattcagattgtaaggacgctgacttagttgttattacagccggtgcgcctcaaaagcctggtgaatcacgtttagacttagttaacaagaatttaaatatcctatcatccattgtcaaaccagttgttgactccggctttgacggcatcttcttagttgctgctaaccctgttgacatcttaacttacgctacttggaaattctcaggtttcccaaaggatcgtgtcattggttcagggacttccttagactcttcacgtttacgcgttgcgttaggcaaacaattcaatgttgatcctcgttccgttgatgcttacatcatgggtgaacacggtgattctgaatttgctgcttactcaactgcaaccatcgggacacgtccagttcgcgatgtcgctaaggaacaaggcgtttctgacgaagatttagccaagttagaagacggtgttcgtaacaaagcgtacgacatcatcaacttgaagggtgccacgttctacggtatcgggactgctttaatgcggatttccaaagccattttacgtgatgaaaatgccgttttaccagtaggtgcctacatggacggccaatacggcttaaacgacatttatatcgggactccggctgtgattggtggaactggtttgaaacaaatcatcgaatcaccactttcagctgacgaactcaagaagatgcaagattccgccgcaactttgaaaaaagtgcttaacgacggtttagctgaattagaaaataaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 56 - DNA, 2454 bp, Artificial
  fusion protein, His-lplLDH::goxDp
atgggcagcagccatcaccatcatcaccacagccaggatccgtcaagcatgccaaatcatcaaaaagttgtgttagtcggcgacggcgctgttggttctagttacgcttttgcgatggcacaacaaggaattgctgaagaatttgtaattgtcgatgttgttaaagatcggacaaagggtgacgcccttgatcttgaagacgcccaagcattcaccgctcccaagaagatttactcaggcgaatattcagattgtaaggacgctgacttagttgttattacagccggtgcgcctcaaaagcctggtgaatcacgtttagacttagttaacaagaatttaaatatcctatcatccattgtcaaaccagttgttgactccggctttgacggcatcttcttagttgctgctaaccctgttgacatcttaacttacgctacttggaaattctcaggtttcccaaaggatcgtgtcattggttcagggacttccttagactcttcacgtttacgcgttgcgttaggcaaacaattcaatgttgatcctcgttccgttgatgcttacatcatgggtgaacacggtgattctgaatttgctgcttactcaactgcaaccatcgggacacgtccagttcgcgatgtcgctaaggaacaaggcgtttctgacgaagatttagccaagttagaagacggtgttcgtaacaaagcgtacgacatcatcaacttgaagggtgccacgttctacggtatcgggactgctttaatgcggatttccaaagccattttacgtgatgaaaatgccgttttaccagtaggtgcctacatggacggccaatacggcttaaacgacatttatatcgggactccggctgtgattggtggaactggtttgaaacaaatcatcgaatcaccactttcagctgacgaactcaagaagatgcaagattccgccgcaactttgaaaaaagtgcttaacgacggtttagctgaattagaaaataaaaagcttgaaggcaaaagcagcggcagcggcagcgaaagcaaaagcacccatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatc

SEQ ID NO: 57 - protein, 818 aa, Artificial
  fusion protein, His-lplLDH::goxDp
MGSSHHHHHHSQDPSSMPNHQKVVLVGDGAVGSSYAFAMAQQGIAEEFVIVDVVKDRTKGDALDLEDAQAFTAPKKIYSGEYSDCKDADLVVITAGAPQKPGESRLDLVNKNLNILSSIVKPVVDSGFDGIFLVAANPVDILTYATWKFSGFPKDRVIGSGTSLDSSRLRVALGKQFNVDPRSVDAYIMGEHGDSEFAAYSTATIGTRPVRDVAKEQGVSDEDLAKLEDGVRNKAYDIINLKGATFYGIGTALMRISKAILRDENAVLPVGAYMDGQYGLNDIYIGTPAVIGGTGLKQIIESPLSADELKKMQDSAATLKKVLNDGLAELENKKLEGKSSGSGSESKSTHMCDTFDLIVVGGGPGGYVAALRASQLGMSVALVESTHFGGVCLNWGCIPTKALLRSSEIHHLLHELGTFGLSADNISFDLSKIVGRSRSIARRMGGGIAHLLKKTKVTTFDGRAKLAGRSGEAHQVAITKDGAAVATIKAPHVILATGARGRQLPGLETDGTLIWGAREAMTPKELPKRLLVIGSGAIGIEFASFYRNMGSEVTIAEVADRILIAEDPEISAAARKAFEKQGMKIITSAKVGPLNKGENEVSTTIESPTGKVDLTVDRVICAVGIVGNVEDLGLEGTKVQVERTHIVTDGFCRTGEPGIYAIGDVAGAPWLAHKASHEGILCVEKIAGRSPQPLHPLNIPGCTYSRPQIASVGLSEEKAIAAGHKVKVGRFPFIANGKAVAMGETDGMVKTVFDATSGELLGAHMIGAEVTEMIQGYVITRTGELTEAELVETVFPHPTISETMHEATLAAFDGPLHI

SEQ ID NO: 58 - DNA, 42 bp, Artificial
  PCR Primer
aataatcatatgtcaagcatgccaaatcatcaaaaagttgtg

SEQ ID NO: 59 - DNA, 42 bp, Artificial
  PCR Primer
aataatctcgagtttattttctaattcagctaaaccgtcgtt

SEQ ID NO: 60 - DNA, 42 bp, Artificial
  PCR Primer
aataatctcgaggatatgcagcgggccgtcgaaggccgccag

SEQ ID NO: 61 - DNA, 43 bp, Artificial
  PCR Primer
aataatggatccgaaaataggtatcgtaggactcggaagggtt

SEQ ID NO: 62 - DNA, 42 bp, Artificial
  PCR Primer
aataataagcttaccgctggtgttctggtgcttgttctcttc

SEQ ID NO: 63 - DNA, 42 bp, Artificial
  PCR Primer
aataatcatatgtacgatgctgtgatcataggaggaggaccc

SEQ ID NO: 64 - DNA, 42 bp, Artificial
  PCR Primer
aataatctcgagtcacagatgaatgggttttcctgagacccc

SEQ ID NO: 65 - DNA, 7534 bp, Artificial
  plasmid, pETDuet-tmaLDH::tmaDp
taatacgactcactataggggaattgtgagcggataacaattcccctctagaaataattttgtttaactttaagaaggagatataccatgggcagcagccatcaccatcatcaccacagccaggatccgaaaataggtatcgtaggactcggaagggttggttccagcacggcttttgcactcctgatgaaaggttttgcgagggaaatggttctgatagacgtcgataagaaaagagcagaaggagacgctctcgatctcattcacggaacacctttcacgagaagagcgaacatctacgctggagactatgcggatctgaaaggatcagatgtggtgatcgtcgctgcgggagtacctcagaaaccaggagagacgaggcttcagcttcttgggagaaatgcaagagtgatgaaagaaatagcgcgaaacgtctccaaatacgctcctgattcgattgtcatcgtggtcacgaatcccgtcgatgtcctcacgtatttcttcctcaaagagtccggaatggaccccaggaaggtgttcggttccggaacggttctcgacacagcaaggctcagaactttgatagcgcaacactgcggcttttctccaagaagtgtgcacgtgtacgtgataggagaacatggagattcagaggttcctgtgtggagcggtgccatgataggaggtatccccctacagaacatgtgtcagatttgtcagaagtgcgattcgaagatactggaaaacttcgctgaaaaaaccaaaagagccgcgtacgagatcatagagagaaagggagccacccattacgccatagctctcgccgtggccgacatagtggagagcatcttcttcgacgaaaagagagtgcttacgctttccgtttatctcgaagattacctcggtgtaaaagatctctgtatcagtgttcctgttacccttggaaagcacggagtggaaagaatcctcgaactgaacctgaacgaagaagaactggaagccttcagaaagtcagcttccatattgaaaaacgccatcaacgaaataacggccgaagagaacaagcaccagaacaccagcggtaagcttgaaggcaaaagcagcggcagcggcagcgaaagcaaaagcacccatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 66 - DNA, 44 bp, Artificial
  PCR Primer
aataatccatggcgataggtatcgtaggactcggaagggttggt

SEQ ID NO: 67 - DNA, 5962 bp, Artificial
  plasmid, pCDFDuet-tmaLDH-tmaDp
taatacgactcactataggggaattgtgagcggataacaattcccctgtagaaataattttgtttaactttaataaggagatataccatggcgataggtatcgtaggactcggaagggttggttccagcacggcttttgcactcctgatgaaaggttttgcgagggaaatggttctgatagacgtcgataagaaaagagcagaaggagacgctctcgatctcattcacggaacacctttcacgagaagagcgaacatctacgctggagactatgcggatctgaaaggatcagatgtggtgatcgtcgctgcgggagtacctcagaaaccaggagagacgaggcttcagcttcttgggagaaatgcaagagtgatgaaagaaatagcgcgaaacgtctccaaatacgctcctgattcgattgtcatcgtggtcacgaatcccgtcgatgtcctcacgtatttcttcctcaaagagtccggaatggaccccaggaaggtgttcggttccggaacggttctcgacacagcaaggctcagaactttgatagcgcaacactgcggcttttctccaagaagtgtgcacgtgtacgtgataggagaacatggagattcagaggttcctgtgtggagcggtgccatgataggaggtatccccctacagaacatgtgtcagatttgtcagaagtgcgattcgaagatactggaaaacttcgctgaaaaaaccaaaagagccgcgtacgagatcatagagagaaagggagccacccattacgccatagctctcgccgtggccgacatagtggagagcatcttcttcgacgaaaagagagtgcttacgctttccgtttatctcgaagattacctcggtgtaaaagatctctgtatcagtgttcctgttacccttggaaagcacggagtggaaagaatcctcgaactgaacctgaacgaagaagaactggaagccttcagaaagtcagcttccatattgaaaaacgccatcaacgaaataacggccgaagagaacaagcaccagaacaccagcggtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 68 - DNA, 2394 bp, Artificial
  fusion protein, His-tmaLDH::tmaDp
atgggcagcagccatcaccatcatcaccacagccaggatccgaaaataggtatcgtaggactcggaagggttggttccagcacggcttttgcactcctgatgaaaggttttgcgagggaaatggttctgatagacgtcgataagaaaagagcagaaggagacgctctcgatctcattcacggaacacctttcacgagaagagcgaacatctacgctggagactatgcggatctgaaaggatcagatgtggtgatcgtcgctgcgggagtacctcagaaaccaggagagacgaggcttcagcttcttgggagaaatgcaagagtgatgaaagaaatagcgcgaaacgtctccaaatacgctcctgattcgattgtcatcgtggtcacgaatcccgtcgatgtcctcacgtatttcttcctcaaagagtccggaatggaccccaggaaggtgttcggttccggaacggttctcgacacagcaaggctcagaactttgatagcgcaacactgcggcttttctccaagaagtgtgcacgtgtacgtgataggagaacatggagattcagaggttcctgtgtggagcggtgccatgataggaggtatccccctacagaacatgtgtcagatttgtcagaagtgcgattcgaagatactggaaaacttcgctgaaaaaaccaaaagagccgcgtacgagatcatagagagaaagggagccacccattacgccatagctctcgccgtggccgacatagtggagagcatcttcttcgacgaaaagagagtgcttacgctttccgtttatctcgaagattacctcggtgtaaaagatctctgtatcagtgttcctgttacccttggaaagcacggagtggaaagaatcctcgaactgaacctgaacgaagaagaactggaagccttcagaaagtcagcttccatattgaaaaacgccatcaacgaaataacggccgaagagaacaagcaccagaacaccagcggtaagcttgaaggcaaaagcagcggcagcggcagcgaaagcaaaagcacccatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctg

SEQ ID NO: 69 - protein, 798 aa, Artificial
  fusion protein, His-tmaLDH::tmaDp
MGSSHHHHHHSQDPKIGIVGLGRVGSSTAFALLMKGFAREMVLIDVDKKRAEGDALDLIHGTPFTRRANIYAGDYADLKGSDVVIVAAGVPQKPGETRLQLLGRNARVMKEIARNVSKYAPDSIVIVVTNPVDVLTYFFLKESGMDPRKVFGSGTVLDTARLRTLIAQHCGFSPRSVHVYVIGEHGDSEVPVWSGAMIGGIPLQNMCQICQKCDSKILENFAEKTKRAAYEIIERKGATHYAIALAVADIVESIFFDEKRVLTLSVYLEDYLGVKDLCISVPVTLGKHGVERILELNLNEEELEAFRKSASILKNAINEITAEENKHQNTSGKLEGKSSGSGSESKSTHMYDAVIIGGGPGGYVCAIKLAQLGKKVALVEKDALGGTCTNRGCIPTKAMLTVSHLMDEMKEKASKYGLKVSGVEYDVAAIMKHVQKSVMMSRKGIEYLLKKNGVEVFKGTAVVENKNTVVVQETGEKLEAKNLVLAHGSVPSVFSPFDIDGVWTSDDVFNLKEFPKSLVIVGGGVIGVEFATFFGSFGVDVTIVEIAEHILPYEDSDVAEEVKKALKRKGVKILEKTKISSLSKVDDGFEVALENGETLKAEKVLLAAGRKPNIPEDVKALGVKIEKGVVTDSRMRTNVENVYAIGDIRSGIMLAHVAMYEGIVAAKNIAGEEEEMDYSAVPSIIFSSPEVASVGVREKDVNPEEVVISKFPVSANGRARTMLENIGFAKVIADKKDGTVLGMSIVSPSATDMIMEGVIAVKFRMKAEDLEKAIHPHPTLTETILGALEGVSGKPIHL

SEQ ID NO: 70 - DNA, 42 bp, Artificial
  PCR Primer
aataatcatatgaaaataggtatcgtaggactcggaagggtt

SEQ ID NO: 71 - DNA, 42 bp, Artificial
  PCR Primer
aataatctcgagaccgctggtgttctggtgcttgttctcttc

SEQ ID NO: 72 - DNA, 42 bp, Artificial
  PCR Primer
aataatctcgagcagatgaatgggttttcctgagaccccttc

SEQ ID NO: 73 - DNA, 43 bp, Artificial
  PCR Primer
aataatggatccgtcagacctgaaaaccgccgctctcgaatat

SEQ ID NO: 74 - DNA, 42 bp, Artificial
  PCR Primer
aataataagcttgccgttgaacacttcatccacgctggtcag

SEQ ID NO: 75 - DNA, 7900 bp, Artificial
  plasmid, pETDuet-ppuMDH::goxDp
taatacgactcactataggggaattgtgagcggataacaattcccctctagaaataattttgtttaactttaagaaggagatataccatgggcagcagccatcaccatcatcaccacagccaggatccgtcagacctgaaaaccgccgctctcgaatatcacgctcaacctcgtccggggaaactgagcgttgaactctccaagcccactgccaccgcccgtgacctcgccctggcctacagcccaggtgttgcagagccggtgcgcgaaattggccgtgatccagagctggcttacaaatacaccggcaaaggcaacctggttgcggtgatttccgatggcactgccatcctcggtctgggtgacctcggcccactggcttccaagccggtcatggaaggcaagggtgttctgttcaagcgtttcgccggtatcgatgtgttcgacatcgaagtcgagtcggaaagcccgcaagcgttcatcgacaccgttcgccgcatttcgatcaccttcggtggcatcaaccttgaagacatcaaggcacctgagtgctttgaaatcgaacgtaccctgatcgaacagtgcgacatcccggtgttccacgatgaccagcacggtactgccatcgtgactgccgctggcatgatcaacgccctggaaatcgccggcaagaaactggaagaagccaagatcgtctgcctgggcgccggcgctgctgccatttcctgcatgaagctgctggtaagcatgggtgcgaaggtcgagaacatcttcatgatcgaccgtagtggcgtgatccacgctggccgtgacgacctgaaccagtacaaggcgcagttcgcccacgcgaccgacaagcgcaccctggccgacgcccttgacggtgccgacgtgttcgtaggtctgtctggcccgaacctgctgagcccggaaggcctgaagtcgatggctgccaacccgatcgtgttcgcctgctcgaacccggacccggaaatctcgccagagctggcgcatgccactcgcaacgacgtgatcatggctaccggtcgttccgactacccgaaccaggtcaacaacgtactgggcttcccgttcatcttccgtggtgctctggacgttcgcgccaagcgtatcaacgaagaaatgaagatcgctgccgctatcgccctgaaagacctggccaagctgccagtgccgaaggaagtttgcgaagcctatggcgttgaaggcctggagttcggtcgtgagtacatcattccgaagccgctggatgcacgcttgatcaccgtcgtttccgatgctgtggccaaggctgccatcgaatccggcgtggctaccctgccttatccgaagcattacccgctgaccagcgtggatgaagtgttcaacggcaagcttgaaggcaaaagcagcggcagcggcagcgaaagcaaaagcacccatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 76 - DNA, 44 bp, Artificial
  PCR Primer
aataatccatggcggacctgaaaaccgccgctctcgaatatcac

SEQ ID NO: 77 - DNA, 6328 bp, Artificial
  plasmid, pCDFDuet-ppuMDH-goxDp
taatacgactcactataggggaattgtgagcggataacaattcccctgtagaaataattttgtttaactttaataaggagatataccatggcggacctgaaaaccgccgctctcgaatatcacgctcaacctcgtccggggaaactgagcgttgaactctccaagcccactgccaccgcccgtgacctcgccctggcctacagcccaggtgttgcagagccggtgcgcgaaattggccgtgatccagagctggcttacaaatacaccggcaaaggcaacctggttgcggtgatttccgatggcactgccatcctcggtctgggtgacctcggcccactggcttccaagccggtcatggaaggcaagggtgttctgttcaagcgtttcgccggtatcgatgtgttcgacatcgaagtcgagtcggaaagcccgcaagcgttcatcgacaccgttcgccgcatttcgatcaccttcggtggcatcaaccttgaagacatcaaggcacctgagtgctttgaaatcgaacgtaccctgatcgaacagtgcgacatcccggtgttccacgatgaccagcacggtactgccatcgtgactgccgctggcatgatcaacgccctggaaatcgccggcaagaaactggaagaagccaagatcgtctgcctgggcgccggcgctgctgccatttcctgcatgaagctgctggtaagcatgggtgcgaaggtcgagaacatcttcatgatcgaccgtagtggcgtgatccacgctggccgtgacgacctgaaccagtacaaggcgcagttcgcccacgcgaccgacaagcgcaccctggccgacgcccttgacggtgccgacgtgttcgtaggtctgtctggcccgaacctgctgagcccggaaggcctgaagtcgatggctgccaacccgatcgtgttcgcctgctcgaacccggacccggaaatctcgccagagctggcgcatgccactcgcaacgacgtgatcatggctaccggtcgttccgactacccgaaccaggtcaacaacgtactgggcttcccgttcatcttccgtggtgctctggacgttcgcgccaagcgtatcaacgaagaaatgaagatcgctgccgctatcgccctgaaagacctggccaagctgccagtgccgaaggaagtttgcgaagcctatggcgttgaaggcctggagttcggtcgtgagtacatcattccgaagccgctggatgcacgcttgatcaccgtcgtttccgatgctgtggccaaggctgccatcgaatccggcgtggctaccctgccttatccgaagcattacccgctgaccagcgtggatgaagtgttcaacggcaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 78 - DNA, 2760 bp, Artificial
  fusion protein, His-ppuMDH::goxDp
atgggcagcagccatcaccatcatcaccacagccaggatccgtcagacctgaaaaccgccgctctcgaatatcacgctcaacctcgtccggggaaactgagcgttgaactctccaagcccactgccaccgcccgtgacctcgccctggcctacagcccaggtgttgcagagccggtgcgcgaaattggccgtgatccagagctggcttacaaatacaccggcaaaggcaacctggttgcggtgatttccgatggcactgccatcctcggtctgggtgacctcggcccactggcttccaagccggtcatggaaggcaagggtgttctgttcaagcgtttcgccggtatcgatgtgttcgacatcgaagtcgagtcggaaagcccgcaagcgttcatcgacaccgttcgccgcatttcgatcaccttcggtggcatcaaccttgaagacatcaaggcacctgagtgctttgaaatcgaacgtaccctgatcgaacagtgcgacatcccggtgttccacgatgaccagcacggtactgccatcgtgactgccgctggcatgatcaacgccctggaaatcgccggcaagaaactggaagaagccaagatcgtctgcctgggcgccggcgctgctgccatttcctgcatgaagctgctggtaagcatgggtgcgaaggtcgagaacatcttcatgatcgaccgtagtggcgtgatccacgctggccgtgacgacctgaaccagtacaaggcgcagttcgcccacgcgaccgacaagcgcaccctggccgacgcccttgacggtgccgacgtgttcgtaggtctgtctggcccgaacctgctgagcccggaaggcctgaagtcgatggctgccaacccgatcgtgttcgcctgctcgaacccggacccggaaatctcgccagagctggcgcatgccactcgcaacgacgtgatcatggctaccggtcgttccgactacccgaaccaggtcaacaacgtactgggcttcccgttcatcttccgtggtgctctggacgttcgcgccaagcgtatcaacgaagaaatgaagatcgctgccgctatcgccctgaaagacctggccaagctgccagtgccgaaggaagtttgcgaagcctatggcgttgaaggcctggagttcggtcgtgagtacatcattccgaagccgctggatgcacgcttgatcaccgtcgtttccgatgctgtggccaaggctgccatcgaatccggcgtggctaccctgccttatccgaagcattacccgctgaccagcgtggatgaagtgttcaacggcaagcttgaaggcaaaagcagcggcagcggcagcgaaagcaaaagcacccatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatc

SEQ ID NO: 79 - protein, 920 aa, Artificial
  fusion protein, His-ppuMDH::goxDp
MGSSHHHHHHSQDPSDLKTAALEYHAQPRPGKLSVELSKPTATARDLALAYSPGVAEPVREIGRDPELAYKYTGKGNLVAVISDGTAILGLGDLGPLASKPVMEGKGVLFKRFAGIDVFDIEVESESPQAFIDTVRRISITFGGINLEDIKAPECFEIERTLIEQCDIPVFHDDQHGTAIVTAAGMINALEIAGKKLEEAKIVCLGAGAAAISCMKLLVSMGAKVENIFMIDRSGVIHAGRDDLNQYKAQFAHATDKRTLADALDGADVFVGLSGPNLLSPEGLKSMAANPIVFACSNPDPEISPELAHATRNDVIMATGRSDYPNQVNNVLGFPFIFRGALDVRAKRINEEMKIAAAIALKDLAKLPVPKEVCEAYGVEGLEFGREYIIPKPLDARLITVVSDAVAKAAIESGVATLPYPKHYPLTSVDEVFNGKLEGKSSGSGSESKSTHMCDTFDLIVVGGGPGGYVAALRASQLGMSVALVESTHFGGVCLNWGCIPTKALLRSSEIHHLLHELGTFGLSADNISFDLSKIVGRSRSIARRMGGGIAHLLKKTKVTTFDGRAKLAGRSGEAHQVAITKDGAAVATIKAPHVILATGARGRQLPGLETDGTLIWGAREAMTPKELPKRLLVIGSGAIGIEFASFYRNMGSEVTIAEVADRILIAEDPEISAAARKAFEKQGMKIITSAKVGPLNKGENEVSTTIESPTGKVDLTVDRVICAVGIVGNVEDLGLEGTKVQVERTHIVTDGFCRTGEPGIYAIGDVAGAPWLAHKASHEGILCVEKIAGRSPQPLHPLNIPGCTYSRPQIASVGLSEEKAIAAGHKVKVGRFPFIANGKAVAMGETDGMVKTVFDATSGELLGAHMIGAEVTEMIQGYVITRTGELTEAELVETVFPHPTISETMHEATLAAFDGPLHI

SEQ ID NO: 80 - DNA, 42 bp, Artificial
  PCR Primer
aataatcatatgtcagacctgaaaaccgccgctctcgaatat

SEQ ID NO: 81 - DNA, 42 bp, Artificial
  PCR Primer
aataatctcgaggccgttgaacacttcatccacgctggtcag

SEQ ID NO: 82 - DNA, 43 bp, Artificial
  PCR Primer
aataatggatccggctaggatcactgaggaacaaagaaggaaa

SEQ ID NO: 83 - DNA, 42 bp, Artificial
  PCR Primer
aataataagcttagtaatgtactgtttccttctttcatttaa

SEQ ID NO: 84 - DNA, 7882 bp, Artificial
  plasmid, pETDuet-pfuMDH::tmaDp
ggggaattgtgagcggataacaattcccctctagaaataattttgtttaactttaagaaggagatataccatgggcagcagccatcaccatcatcaccacagccaggatccggctaggatcactgaggaacaaagaaggaaacttgctcaagatgcgttggagtatcacagaaataatttcccagggaatggaaaaattgaagtgataccaaaggttcctcttagagatttctatgatttgagcttagcatataccccaggagtagcggagccatgtaaagtaattgcaaagaagggaaactatgaagattacactataattccaaatacagttgccgtcgttacagacggatcggcaatcttgggattaggaaacataggtgttctggcaggaatgccagttatggaaggtaaatgtgttctctttaaagctctagctggaatagatgcgtttcctatcttaattgacacacaggatgtcgagaaaatcgtggaaacaataaaacttatatcaaagggatttggcgggataaacctagaagatatctccgctccaagatgcttcgaaattgaaatgaggttgaagagagagcttgatattcccgtatttcacgatgatcaacatggaactgctgtagtaactttagcgggtctaattaacgcattaaagttagtgggcaaaaaattcaacgagataaaagttgctataagtggagctggagctgctggaatcgcaatagcaaagttgctccatcatgttggggttaaagaaatattgcttgttgatagagctggaataatctatgaagggagaaaagaaaacatgaatccttacaaagaggaagtcgcaaggttcaacattcatggggttcaaggagatttggcaaaagctatggagggtgctgatgtttttattggagtaagcgtaggaggaatagtaaaaccctggatgataaagaaaatggccgatgatcccatagtgtttgcaatggcgaatccaattccagaaattatgccagaagatgcgaaaaaggctggggcaaaaatagtggccacggggaggagtgactatccaaaccaaataaacaatgtcttgggctttcctgggatatttaggggggcactagatgttagggctaccgatataactttaaatatgaacattgcagcggctgaggcaatagcatctgttgtgagcgatgaagaacttagtgaggactacataataccctctccactacatcccgacgtttatccaaaagaagccagagcagttgcagaacaagcaattagggatggtgtagctagaagaaagctatctggggagtgggtagaagagcatactaggaagctccgtgaattttatgccgcactcattgagcccttaaatgaaagaaggaaacagtacattactaagcttgaaggcaaaagcagcggcagcggcagcgaaagcaaaagcacccatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 85 - DNA, 44 bp, Artificial
  PCR Primer
aataatccatggcgaggatcactgaggaacaaagaaggaaactt

SEQ ID NO: 86 - DNA, 6310 bp, Artificial
  plasmid, pCDFDuet-pfuMDH-tmaDp
taatacgactcactataggggaattgtgagcggataacaattcccctgtagaaataattttgtttaactttaataaggagatataccatggcgaggatcactgaggaacaaagaaggaaacttgctcaagatgcgttggagtatcacagaaataatttcccagggaatggaaaaattgaagtgataccaaaggttcctcttagagatttctatgatttgagcttagcatataccccaggagtagcggagccatgtaaagtaattgcaaagaagggaaactatgaagattacactataattccaaatacagttgccgtcgttacagacggatcggcaatcttgggattaggaaacataggtgttctggcaggaatgccagttatggaaggtaaatgtgttctctttaaagctctagctggaatagatgcgtttcctatcttaattgacacacaggatgtcgagaaaatcgtggaaacaataaaacttatatcaaagggatttggcgggataaacctagaagatatctccgctccaagatgcttcgaaattgaaatgaggttgaagagagagcttgatattcccgtatttcacgatgatcaacatggaactgctgtagtaactttagcgggtctaattaacgcattaaagttagtgggcaaaaaattcaacgagataaaagttgctataagtggagctggagctgctggaatcgcaatagcaaagttgctccatcatgttggggttaaagaaatattgcttgttgatagagctggaataatctatgaagggagaaaagaaaacatgaatccttacaaagaggaagtcgcaaggttcaacattcatggggttcaaggagatttggcaaaagctatggagggtgctgatgtttttattggagtaagcgtaggaggaatagtaaaaccctggatgataaagaaaatggccgatgatcccatagtgtttgcaatggcgaatccaattccagaaattatgccagaagatgcgaaaaaggctggggcaaaaatagtggccacggggaggagtgactatccaaaccaaataaacaatgtcttgggctttcctgggatatttaggggggcactagatgttagggctaccgatataactttaaatatgaacattgcagcggctgaggcaatagcatctgttgtgagcgatgaagaacttagtgaggactacataataccctctccactacatcccgacgtttatccaaaagaagccagagcagttgcagaacaagcaattagggatggtgtagctagaagaaagctatctggggagtgggtagaagagcatactaggaagctccgtgaattttatgccgcactcattgagcccttaaatgaaagaaggaaacagtacattactaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 87 - DNA, 2742 bp, Artificial
  fusion protein, His-pfuMDH::tmaDp
atgggcagcagccatcaccatcatcaccacagccaggatccggctaggatcactgaggaacaaagaaggaaacttgctcaagatgcgttggagtatcacagaaataatttcccagggaatggaaaaattgaagtgataccaaaggttcctcttagagatttctatgatttgagcttagcatataccccaggagtagcggagccatgtaaagtaattgcaaagaagggaaactatgaagattacactataattccaaatacagttgccgtcgttacagacggatcggcaatcttgggattaggaaacataggtgttctggcaggaatgccagttatggaaggtaaatgtgttctctttaaagctctagctggaatagatgcgtttcctatcttaattgacacacaggatgtcgagaaaatcgtggaaacaataaaacttatatcaaagggatttggcgggataaacctagaagatatctccgctccaagatgcttcgaaattgaaatgaggttgaagagagagcttgatattcccgtatttcacgatgatcaacatggaactgctgtagtaactttagcgggtctaattaacgcattaaagttagtgggcaaaaaattcaacgagataaaagttgctataagtggagctggagctgctggaatcgcaatagcaaagttgctccatcatgttggggttaaagaaatattgcttgttgatagagctggaataatctatgaagggagaaaagaaaacatgaatccttacaaagaggaagtcgcaaggttcaacattcatggggttcaaggagatttggcaaaagctatggagggtgctgatgtttttattggagtaagcgtaggaggaatagtaaaaccctggatgataaagaaaatggccgatgatcccatagtgtttgcaatggcgaatccaattccagaaattatgccagaagatgcgaaaaaggctggggcaaaaatagtggccacggggaggagtgactatccaaaccaaataaacaatgtcttgggctttcctgggatatttaggggggcactagatgttagggctaccgatataactttaaatatgaacattgcagcggctgaggcaatagcatctgttgtgagcgatgaagaacttagtgaggactacataataccctctccactacatcccgacgtttatccaaaagaagccagagcagttgcagaacaagcaattagggatggtgtagctagaagaaagctatctggggagtgggtagaagagcatactaggaagctccgtgaattttatgccgcactcattgagcccttaaatgaaagaaggaaacagtacattactaagcttgaaggcaaaagcagcggcagcggcagcgaaagcaaaagcacccatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctg

SEQ ID NO: 88 - protein, 914 aa, Artificial
  fusion protein, His-pfuMDH::tmaDp
MGSSHHHHHHSQDPARITEEQRRKLAQDALEYHRNNFPGNGKIEVIPKVPLRDFYDLSLAYTPGVAEPCKVIAKKGNYEDYTIIPNTVAVVTDGSAILGLGNIGVLAGMPVMEGKCVLFKALAGIDAFPILIDTQDVEKIVETIKLISKGFGGINLEDISAPRCFEIEMRLKRELDIPVFHDDQHGTAVVTLAGLINALKLVGKKFNEIKVAISGAGAAGIAIAKLLHHVGVKEILLVDRAGIIYEGRKENMNPYKEEVARFNIHGVQGDLAKAMEGADVFIGVSVGGIVKPWMIKKMADDPIVFAMANPIPEIMPEDAKKAGAKIVATGRSDYPNQINNVLGFPGIFRGALDVRATDITLNMNIAAAEAIASVVSDEELSEDYIIPSPLHPDVYPKEARAVAEQAIRDGVARRKLSGEWVEEHTRKLREFYAALIEPLNERRKQYITKLEGKSSGSGSESKSTHMYDAVIIGGGPGGYVCAIKLAQLGKKVALVEKDALGGTCTNRGCIPTKAMLTVSHLMDEMKEKASKYGLKVSGVEYDVAAIMKHVQKSVMMSRKGIEYLLKKNGVEVFKGTAVVENKNTVVVQETGEKLEAKNLVLAHGSVPSVFSPFDIDGVWTSDDVFNLKEFPKSLVIVGGGVIGVEFATFFGSFGVDVTIVEIAEHILPYEDSDVAEEVKKALKRKGVKILEKTKISSLSKVDDGFEVALENGETLKAEKVLLAAGRKPNIPEDVKALGVKIEKGVVTDSRMRTNVENVYAIGDIRSGIMLAHVAMYEGIVAAKNIAGEEEEMDYSAVPSIIFSSPEVASVGVREKDVNPEEVVISKFPVSANGRARTMLENIGFAKVIADKKDGTVLGMSIVSPSATDMIMEGVIAVKFRMKAEDLEKAIHPHPTLTETILGALEGVSGKPIHL

SEQ ID NO: 89 - DNA, 43 bp, Artificial
  PCR Primer
aataatcatatggctaggatcactgaggaacaaagaaggaaat

SEQ ID NO: 90 - DNA, 42 bp, Artificial
  PCR Primer
aataatctcgagagtaatgtactgtttccttctttcatttaa

SEQ ID NO: 91 - DNA, 43 bp, Artificial
  PCR Primer
aataatggatccgtcttcgcaatcgcagtccccgtccgtcgcg

SEQ ID NO: 92 - DNA, 42 bp, Artificial
  PCR Primer
aataataagcttggggtacagaccgcgcatttcgcgcgccat

SEQ ID NO: 93 - DNA, 7936 bp, Artificial
  plasmid, pETDuet-bmaEDH::goxDp
ggggaattgtgagcggataacaattcccctctagaaataattttgtttaactttaagaaggagatataccatgggcagcagccatcaccatcatcaccacagccaggatccgtcttcgcaatcgcagtccccgtccgtcgcgcagtccattccgtcgtacctgcacgcggacgatctcggcccctggggcaactatttgcagcaagtcgatcgcgtcgcgccgtatctcggctcgctgtcgcgctggatcgagacgctcaagcgcccgaaacgcatcctgatcgtcgacgtgccgatcgagctggacaacggcaccgtcgcgcacttcgagggctaccgcgtgcagcacaacgtgtcgcgcggcccgggcaagggcggcgtgcgctaccaccaggacgtgacgctgtccgaggtgatggcgctgtccgcatggatgtcggtgaagaacgcggccgtgaacgtgccgtacggcggcgcgaagggcggtatccgcgtcgatccgcgcaagctctcgcgcggcgagctggagcgcgtgacgcgccgctacacgagcgaaatcggcatcatcatcggcccgaacaccgatatccccgcgcctgacgtcaacacgaacgagcagatcatggcgtggatgatggacacgtactcgatgaaccagggccagacggccacgggcgtcgtgaccggcaagccgatctcgctcggtggctcgctcggccgcaaggaagcgacgggccgcggcgtgttcgtcgtcggctgcgaggcggcgaagaagaaggggctggagatcgaaggcgcgcgcatcgcggtccagggcttcggcaatgtcggcgggatcgccgcgaagctgttccaggaagcgggcgcgaaggtgatcgcggtgcaggatcacacgggcacgattcaccagccggcgggcgtcgatacggtcaagctgctggagcacgtcggccgcacgggcggcgtcgcgggcttcgaaggcgcggaaccgatgccgaacgacgagttctggaccgtcgaaaccgatatcctgattccggccgcgctggaaaaccagatcaccgagaagaacgcgtcgaagatccgcacgaagatcatcgtcgagggcgcgaacggcccgacgacgacggccgcggacgatatcctgagcgcgaacggcgtgctcgtgatccccgacgtgatcgcgaacgcgggcggcgtgaccgtgtcgtatttcgaatgggtgcaggatttctggagcttcttctggacggaggacgagatcaatcaccgtctggagcgcgtgatgcgcgaggcgttcgccggcgtatgggcggtggccgaagagcacaacgtgtcggtgcgcacggcggcgttcatcgtcgcgtgcaagcgcatcctgatggcgcgcgaaatgcgcggtctgtaccccaagcttgaaggcaaaagcagcggcagcggcagcgaaagcaaaagcacccatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 94 - DNA, 44 bp, Artificial
  PCR Primer
aataatccatggcgtcgcaatcgcagtccccgtccgtcgcgcag

SEQ ID NO: 95 - DNA, 6364 bp, Artificial
  plasmid, pCDFDuet-bmaEDH-goxDp
taatacgactcactataggggaattgtgagcggataacaattcccctgtagaaataattttgtttaactttaataaggagatataccatggcgtcgcaatcgcagtccccgtccgtcgcgcagtccattccgtcgtacctgcacgcggacgatctcggcccctggggcaactatttgcagcaagtcgatcgcgtcgcgccgtatctcggctcgctgtcgcgctggatcgagacgctcaagcgcccgaaacgcatcctgatcgtcgacgtgccgatcgagctggacaacggcaccgtcgcgcacttcgagggctaccgcgtgcagcacaacgtgtcgcgcggcccgggcaagggcggcgtgcgctaccaccaggacgtgacgctgtccgaggtgatggcgctgtccgcatggatgtcggtgaagaacgcggccgtgaacgtgccgtacggcggcgcgaagggcggtatccgcgtcgatccgcgcaagctctcgcgcggcgagctggagcgcgtgacgcgccgctacacgagcgaaatcggcatcatcatcggcccgaacaccgatatccccgcgcctgacgtcaacacgaacgagcagatcatggcgtggatgatggacacgtactcgatgaaccagggccagacggccacgggcgtcgtgaccggcaagccgatctcgctcggtggctcgctcggccgcaaggaagcgacgggccgcggcgtgttcgtcgtcggctgcgaggcggcgaagaagaaggggctggagatcgaaggcgcgcgcatcgcggtccagggcttcggcaatgtcggcgggatcgccgcgaagctgttccaggaagcgggcgcgaaggtgatcgcggtgcaggatcacacgggcacgattcaccagccggcgggcgtcgatacggtcaagctgctggagcacgtcggccgcacgggcggcgtcgcgggcttcgaaggcgcggaaccgatgccgaacgacgagttctggaccgtcgaaaccgatatcctgattccggccgcgctggaaaaccagatcaccgagaagaacgcgtcgaagatccgcacgaagatcatcgtcgagggcgcgaacggcccgacgacgacggccgcggacgatatcctgagcgcgaacggcgtgctcgtgatccccgacgtgatcgcgaacgcgggcggcgtgaccgtgtcgtatttcgaatgggtgcaggatttctggagcttcttctggacggaggacgagatcaatcaccgtctggagcgcgtgatgcgcgaggcgttcgccggcgtatgggcggtggccgaagagcacaacgtgtcggtgcgcacggcggcgttcatcgtcgcgtgcaagcgcatcctgatggcgcgcgaaatgcgcggtctgtaccccaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 96 - DNA, 2796 bp, Artificial
  fusion protein, His-bmaEDH::goxDp
atgggcagcagccatcaccatcatcaccacagccaggatccgtcttcgcaatcgcagtccccgtccgtcgcgcagtccattccgtcgtacctgcacgcggacgatctcggcccctggggcaactatttgcagcaagtcgatcgcgtcgcgccgtatctcggctcgctgtcgcgctggatcgagacgctcaagcgcccgaaacgcatcctgatcgtcgacgtgccgatcgagctggacaacggcaccgtcgcgcacttcgagggctaccgcgtgcagcacaacgtgtcgcgcggcccgggcaagggcggcgtgcgctaccaccaggacgtgacgctgtccgaggtgatggcgctgtccgcatggatgtcggtgaagaacgcggccgtgaacgtgccgtacggcggcgcgaagggcggtatccgcgtcgatccgcgcaagctctcgcgcggcgagctggagcgcgtgacgcgccgctacacgagcgaaatcggcatcatcatcggcccgaacaccgatatccccgcgcctgacgtcaacacgaacgagcagatcatggcgtggatgatggacacgtactcgatgaaccagggccagacggccacgggcgtcgtgaccggcaagccgatctcgctcggtggctcgctcggccgcaaggaagcgacgggccgcggcgtgttcgtcgtcggctgcgaggcggcgaagaagaaggggctggagatcgaaggcgcgcgcatcgcggtccagggcttcggcaatgtcggcgggatcgccgcgaagctgttccaggaagcgggcgcgaaggtgatcgcggtgcaggatcacacgggcacgattcaccagccggcgggcgtcgatacggtcaagctgctggagcacgtcggccgcacgggcggcgtcgcgggcttcgaaggcgcggaaccgatgccgaacgacgagttctggaccgtcgaaaccgatatcctgattccggccgcgctggaaaaccagatcaccgagaagaacgcgtcgaagatccgcacgaagatcatcgtcgagggcgcgaacggcccgacgacgacggccgcggacgatatcctgagcgcgaacggcgtgctcgtgatccccgacgtgatcgcgaacgcgggcggcgtgaccgtgtcgtatttcgaatgggtgcaggatttctggagcttcttctggacggaggacgagatcaatcaccgtctggagcgcgtgatgcgcgaggcgttcgccggcgtatgggcggtggccgaagagcacaacgtgtcggtgcgcacggcggcgttcatcgtcgcgtgcaagcgcatcctgatggcgcgcgaaatgcgcggtctgtaccccaagcttgaaggcaaaagcagcggcagcggcagcgaaagcaaaagcacccatatgtgcgatacgttcgatctgattgttgttggtggcggccccggcggctatgtggcggcgctgcgggccagccagcttggcatgagcgtggcgctcgtggaaagcacgcatttcggcggcgtgtgcctcaactggggctgcattccgaccaaggcgcttctacggtcttcggaaattcaccacctgcttcacgaactggggacgttcggactttccgccgacaatatctcgttcgacctgtccaagatcgttggccgttcacgcagcatcgcacgccggatgggtggcggcattgcacatctgctgaagaagaccaaggtcacgacctttgacggtcgtgcaaagctggccggccgttcgggtgaggcgcatcaggttgccatcaccaaggatggcgcagcagttgccaccatcaaggcgccgcatgtcatccttgcaacgggtgcgcgcggtcgtcagcttccgggtcttgagacagacggcaccctgatctggggcgcacgcgaggccatgacaccgaaggaactccccaagcggctcctcgtcatcggttcgggcgctattggcatcgagttcgcatccttctaccgaaacatgggcagcgaggtcacgattgccgaagtggcggaccggatcctgattgccgaagatccggaaatcagcgccgctgcccgcaaggcttttgagaagcaggggatgaagatcatcaccagcgcgaaggtcggaccgctgaacaagggtgagaacgaggtctccacgacgatcgagagcccgaccgggaaggtcgatctgacggtggatcgcgttatctgcgcggttggcattgtcggcaatgtcgaggatctggggcttgagggcaccaaggttcaggtcgagcggacccatatcgtgacggacggcttctgccgcaccggcgaaccgggcatctatgcgatcggcgacgttgcgggtgctccgtggctggcacacaaggcgagccatgaaggcatcctgtgcgtcgagaagattgctggccgttcgccgcagccgctccatccgctgaatatcccgggctgtacctattcgcgtccgcagatcgcctctgtcgggcttagcgaagagaaggccattgctgccggccacaaggtcaaggtcggccgcttcccgttcatcgccaacggcaaggccgtcgcgatgggtgagacggacggcatggtcaaaaccgtgttcgatgccacgagcggcgaacttcttggcgcccacatgatcggcgcggaagtcacggaaatgatccaaggctacgtcatcacccgcacaggcgaactgaccgaagcggagctggtcgagacggttttcccgcatccgaccatttccgaaaccatgcatgaggcaactctggcggccttcgacggcccgctgcatatc

SEQ ID NO: 97 - protein, 932 aa, Artificial
  fusion protein, His-bmaEDH::goxDp
MGSSHHHHHHSQDPSSQSQSPSVAQSIPSYLHADDLGPWGNYLQQVDRVAPYLGSLSRWIETLKRPKRILIVDVPIELDNGTVAHFEGYRVQHNVSRGPGKGGVRYHQDVTLSEVMALSAWMSVKNAAVNVPYGGAKGGIRVDPRKLSRGELERVTRRYTSEIGIIIGPNTDIPAPDVNTNEQIMAWMMDTYSMNQGQTATGVVTGKPISLGGSLGRKEATGRGVFVVGCEAAKKKGLEIEGARIAVQGFGNVGGIAAKLFQEAGAKVIAVQDHTGTIHQPAGVDTVKLLEHVGRTGGVAGFEGAEPMPNDEFWTVETDILIPAALENQITEKNASKIRTKIIVEGANGPTTTAADDILSANGVLVIPDVIANAGGVTVSYFEWVQDFWSFFWTEDEINHRLERVMREAFAGVWAVAEEHNVSVRTAAFIVACKRILMAREMRGLYPKLEGKSSGSGSESKSTHMCDTFDLIVVGGGPGGYVAALRASQLGMSVALVESTHFGGVCLNWGCIPTKALLRSSEIHHLLHELGTFGLSADNISFDLSKIVGRSRSIARRMGGGIAHLLKKTKVTTFDGRAKLAGRSGEAHQVAITKDGAAVATIKAPHVILATGARGRQLPGLETDGTLIWGAREAMTPKELPKRLLVIGSGAIGIEFASFYRNMGSEVTIAEVADRILIAEDPEISAAARKAFEKQGMKIITSAKVGPLNKGENEVSTTIESPTGKVDLTVDRVICAVGIVGNVEDLGLEGTKVQVERTHIVTDGFCRTGEPGIYAIGDVAGAPWLAHKASHEGILCVEKIAGRSPQPLHPLNIPGCTYSRPQIASVGLSEEKAIAAGHKVKVGRFPFIANGKAVAMGETDGMVKTVFDATSGELLGAHMIGAEVTEMIQGYVITRTGELTEAELVETVFPHPTISETMHEATLAAFDGPLHI

SEQ ID NO: 98 - DNA, 42 bp, Artificial
  PCR Primer
aataatcatatgtcttcgcaatcgcagtccccgtccgtcgcg

SEQ ID NO: 99 - DNA, 42 bp, Artificial
  PCR Primer
aataatctcgagggggtacagaccgcgcatttcgcgcgccat

SEQ ID NO: 100 - DNA, 43 bp, Artificial
  PCR Primer
aataatggatccggttgagcaagacccctatgaaattgttatt

SEQ ID NO: 101 - DNA, 42 bp, Artificial
  PCR Primer
aataataagcttgtgcttgacccatccacggtcaagcattgc

SEQ ID NO: 102 - DNA, 7837 bp, Artificial
  plasmid, pETDuet-pfuEDH::tmaDp
ggggaattgtgagcggataacaattcccctctagaaataattttgtttaactttaagaaggagatataccatgggcagcagccatcaccatcatcaccacagccaggatccggttgagcaagacccctatgaaattgttattaagcaacttgaaagagctgcccaatatatggagataagtgaagaagctcttgagttcttaaagagacctcaaagaattgttgaggtcacaattccagtagaaatggatgacggttctgtaaaagttttcactggatttagagtacaacacaactgggctagaggtccaactaagggtggaattagatggcatccagaagaaacccttagcactgttaaagctcttgcagcttggatgacatggaagactgctgtaatggatctcccgtatggtggaggtaagggtggaataattgtagatccaaagaagctctccgacagagagaaggagaggcttgcaagaggttacattagagcaatttatgatgttattagcccgtatgaagacattccagcacccgatgtttatacaaacccacaaataatggcatggatgatggatgagtacgagacaataagcaggagaaagacaccggcctttggaattatcactggaaagcctcttagcattggtggatcacttggaagaattgaggcaactgcaagaggtgcaagttacacaattagagaggctgcaaaggttcttggatgggacaccctcaagggcaagacaatagcaatccagggttacggtaacgcgggttattatcttgcaaagatcatgagtgaagactttggaatgaaggttgtagctgtgagcgacagcaagggtggaatatacaaccccgatggtcttaatgctgacgaggttctcaagtggaagaatgagcatggaagcgttaaagacttcccaggagcaaccaacataacgaatgaggagctacttgagcttgaggttgatgttctcgctccggcagctatagaagaagtgataactaagaagaacgcagacaacattaaggctaagatcgttgcagaagtagcaaacggtccagttactccagaagctgatgagatactattcgagaaaggaatccttcagatcccagacttcctatgtaatgctggtggagttacagtcagctacttcgagtgggtacagaacataactggatactactggacaattgaggaggttagagagagactcgacaagaagatgacaaaagcattctacgacgtctacaacatagcaaaggagaagaacatacacatgagagatgcagcttacgtagttgcagtccagagagtttatcaagcaatgcttgaccgtggatgggtcaagcacaagcttgaaggcaaaagcagcggcagcggcagcgaaagcaaaagcacccatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 103 - DNA, 44 bp, Artificial
  PCR Primer
aataatccatggcggagcaagacccctatgaaattgttattaag

SEQ ID NO: 104 - DNA, 6265 bp, Artificial
  plasmid, pCDFDuet-pfuEDH-tmaDp
taatacgactcactataggggaattgtgagcggataacaattcccctgtagaaataattttgtttaactttaataaggagatataccatggcggagcaagacccctatgaaattgttattaagcaacttgaaagagctgcccaatatatggagataagtgaagaagctcttgagttcttaaagagacctcaaagaattgttgaggtcacaattccagtagaaatggatgacggttctgtaaaagttttcactggatttagagtacaacacaactgggctagaggtccaactaagggtggaattagatggcatccagaagaaacccttagcactgttaaagctcttgcagcttggatgacatggaagactgctgtaatggatctcccgtatggtggaggtaagggtggaataattgtagatccaaagaagctctccgacagagagaaggagaggcttgcaagaggttacattagagcaatttatgatgttattagcccgtatgaagacattccagcacccgatgtttatacaaacccacaaataatggcatggatgatggatgagtacgagacaataagcaggagaaagacaccggcctttggaattatcactggaaagcctcttagcattggtggatcacttggaagaattgaggcaactgcaagaggtgcaagttacacaattagagaggctgcaaaggttcttggatgggacaccctcaagggcaagacaatagcaatccagggttacggtaacgcgggttattatcttgcaaagatcatgagtgaagactttggaatgaaggttgtagctgtgagcgacagcaagggtggaatatacaaccccgatggtcttaatgctgacgaggttctcaagtggaagaatgagcatggaagcgttaaagacttcccaggagcaaccaacataacgaatgaggagctacttgagcttgaggttgatgttctcgctccggcagctatagaagaagtgataactaagaagaacgcagacaacattaaggctaagatcgttgcagaagtagcaaacggtccagttactccagaagctgatgagatactattcgagaaaggaatccttcagatcccagacttcctatgtaatgctggtggagttacagtcagctacttcgagtgggtacagaacataactggatactactggacaattgaggaggttagagagagactcgacaagaagatgacaaaagcattctacgacgtctacaacatagcaaaggagaagaacatacacatgagagatgcagcttacgtagttgcagtccagagagtttatcaagcaatgcttgaccgtggatgggtcaagcacaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctgtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 105 - DNA, 2697 bp, Artificial
  fusion protein, His-pfuEDH::tmaDp
atgggcagcagccatcaccatcatcaccacagccaggatccggttgagcaagacccctatgaaattgttattaagcaacttgaaagagctgcccaatatatggagataagtgaagaagctcttgagttcttaaagagacctcaaagaattgttgaggtcacaattccagtagaaatggatgacggttctgtaaaagttttcactggatttagagtacaacacaactgggctagaggtccaactaagggtggaattagatggcatccagaagaaacccttagcactgttaaagctcttgcagcttggatgacatggaagactgctgtaatggatctcccgtatggtggaggtaagggtggaataattgtagatccaaagaagctctccgacagagagaaggagaggcttgcaagaggttacattagagcaatttatgatgttattagcccgtatgaagacattccagcacccgatgtttatacaaacccacaaataatggcatggatgatggatgagtacgagacaataagcaggagaaagacaccggcctttggaattatcactggaaagcctcttagcattggtggatcacttggaagaattgaggcaactgcaagaggtgcaagttacacaattagagaggctgcaaaggttcttggatgggacaccctcaagggcaagacaatagcaatccagggttacggtaacgcgggttattatcttgcaaagatcatgagtgaagactttggaatgaaggttgtagctgtgagcgacagcaagggtggaatatacaaccccgatggtcttaatgctgacgaggttctcaagtggaagaatgagcatggaagcgttaaagacttcccaggagcaaccaacataacgaatgaggagctacttgagcttgaggttgatgttctcgctccggcagctatagaagaagtgataactaagaagaacgcagacaacattaaggctaagatcgttgcagaagtagcaaacggtccagttactccagaagctgatgagatactattcgagaaaggaatccttcagatcccagacttcctatgtaatgctggtggagttacagtcagctacttcgagtgggtacagaacataactggatactactggacaattgaggaggttagagagagactcgacaagaagatgacaaaagcattctacgacgtctacaacatagcaaaggagaagaacatacacatgagagatgcagcttacgtagttgcagtccagagagtttatcaagcaatgcttgaccgtggatgggtcaagcacaagcttgaaggcaaaagcagcggcagcggcagcgaaagcaaaagcacccatatgtacgatgctgtgatcataggaggaggacccggaggttacgtctgcgctataaaactcgcacagcttggaaagaaggtcgcacttgtcgaaaaagatgccctcgggggaacctgtaccaacaggggatgcattccaacgaaggcgatgctcacagtctcccatctcatggatgaaatgaaagagaaagcgtcaaagtacggcttgaaagtctcaggagtcgaatacgacgtcgccgccataatgaaacacgttcaaaagagtgtgatgatgtcaagaaagggaatagagtacctgctgaaaaagaatggggttgaagtcttcaaaggaacggccgtcgtggaaaacaagaacacggttgttgtccaggaaacgggagagaaactggaagcaaagaatcttgttctcgcacacggatccgttccctccgtgttttctcccttcgatatcgacggagtctggacgagtgatgacgtgttcaatttgaaagaatttccaaagtcgcttgtgatagtgggcggtggtgtcatcggagttgaattcgcgacgtttttcggttctttcggtgtggatgtgacgatcgttgagatcgcagaacacatcctcccctatgaagactcagatgtggcagaagaagtgaaaaaggccctcaaaaggaagggtgttaagatcctggaaaaaacaaaaatttcctccctgagcaaagtcgatgacggctttgaagtcgcactcgaaaatggagaaacccttaaggcagaaaaggtcctgctcgccgcaggaagaaagcccaacataccggaggacgtgaaagctctcggagtgaagatagaaaaaggagtcgtcactgatagcaggatgagaacgaacgtggaaaacgtttacgcgataggagatatcagaagtggaatcatgctcgctcatgtggccatgtacgagggaatagtcgctgcaaagaacatcgcgggagaagaggaagaaatggattactcggccgttccgagtatcatcttctcctctccggaagtggcatctgtcggtgtgagagaaaaagacgtgaaccccgaagaagttgtgatctccaagttcccagtctccgcaaacggaagggcaagaacgatgcttgaaaacatagggtttgccaaggtcatagcggacaagaaagacggaacggttctcggcatgagcatcgtttcaccatcggctaccgatatgatcatggaaggtgtcatagccgtgaagttcaggatgaaagcggaagatctggagaaagccattcacccgcatccgactttgacagaaaccatactcggggctcttgaaggggtctcaggaaaacccattcatctg

SEQ ID NO: 106 - protein, 899 aa, Artificial
  fusion protein, His-pfuEDH::tmaDp
MGSSHHHHHHSQDPVEQDPYEIVIKQLERAAQYMEISEEALEFLKRPQRIVEVTIPVEMDDGSVKVFTGFRVQHNWARGPTKGGIRWHPEETLSTVKALAAWMTWKTAVMDLPYGGGKGGIIVDPKKLSDREKERLARGYIRAIYDVISPYEDIPAPDVYTNPQIMAWMMDEYETISRRKTPAFGIITGKPLSIGGSLGRIEATARGASYTIREAAKVLGWDTLKGKTIAIQGYGNAGYYLAKIMSEDFGMKVVAVSDSKGGIYNPDGLNADEVLKWKNEHGSVKDFPGATNITNEELLELEVDVLAPAAIEEVITKKNADNIKAKIVAEVANGPVTPEADEILFEKGILQIPDFLCNAGGVTVSYFEWVQNITGYYWTIEEVRERLDKKMTKAFYDVYNIAKEKNIHMRDAAYVVAVQRVYQAMLDRGWVKHKLEGKSSGSGSESKSTHMYDAVIIGGGPGGYVCAIKLAQLGKKVALVEKDALGGTCTNRGCIPTKAMLTVSHLMDEMKEKASKYGLKVSGVEYDVAAIMKHVQKSVMMSRKGIEYLLKKNGVEVFKGTAVVENKNTVVVQETGEKLEAKNLVLAHGSVPSVFSPFDIDGVWTSDDVFNLKEFPKSLVIVGGGVIGVEFATFFGSFGVDVTIVEIAEHILPYEDSDVAEEVKKALKRKGVKILEKTKISSLSKVDDGFEVALENGETLKAEKVLLAAGRKPNIPEDVKALGVKIEKGVVTDSRMRTNVENVYAIGDIRSGIMLAHVAMYEGIVAAKNIAGEEEEMDYSAVPSIIFSSPEVASVGVREKDVNPEEVVISKFPVSANGRARTMLENIGFAKVIADKKDGTVLGMSIVSPSATDMIMEGVIAVKFRMKAEDLEKAIHPHPTLTETILGALEGVSGKPIHL

SEQ ID NO: 107 - DNA, 42 bp, Artificial
  PCR Primer
aataatcatatggttgagcaagacccctatgaaattgttatt

SEQ ID NO: 108 - DNA, 42 bp, Artificial
  PCR Primer
aataatctcgaggtgcttgacccatccacggtcaagcattgc

SEQ ID NO: 109 - DNA, 42 bp, Artificial
  PCR Primer
aataatcatatgacggcatctctccgttgcgaagtggcagcg

SEQ ID NO: 110 - DNA, 42 bp, Artificial
  PCR Primer
aataatctcgagtcaggcgtccgccgaaccgccatacacatc

SEQ ID NO: 111 - DNA, 7807 bp, Artificial
  plasmid, pETDuet-sceADH::goxALDH
ggggaattgtgagcggataacaattcccctctagaaataattttgtttaactttaagaaggagatataccatgggcagcagccatcaccatcatcaccacagccaggatccgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttagcggcggcagcggcagcggcggcagcggccatatgacggcatctctccgttgcgaagtggcagcgctcctcgacagccttggcgtgtcacctgatctgtatacggggggcagtctggcggtgaagtctccgctgacgggagaggtgattgccgaggtctcggaagtcagcgcggagcaggcgaaggagacgatcgccagttcactcgacgcgttcaaagcctggcgcagggttccggctccgcgtcgcggtgagcttgtccgcctgctgggtgaggagcttcgggccagcaaggaggcgctcggacggctcgtgacgctggaagtgggcaaggtgccgtccgaaggtctgggcgaagtgcaggaaatgatcgatatctgcgacttcgcggtcggtctgtcccgtcagctttatggcctgacgatccagtccgaacgtccggatcatcgcctgacagagcagtggcatcccgccgggccggtgggcatcatttcggcattcaatttccccgtggccgtctggtcctggaatgctgcgctggcactggtttgcggtgacagcgtgatctggaaaccttcggaaaagaccccgctgacagcgctggccacgcaggccctgttcctgaaagccgctgcccgtttcggcagtgacgcaccggtcgccctcagcacgcttctggtcggtggccgcgagatcggcgaactgatggtgaaagaccgtcgtgtccctgtcatctcggcgacgggttcgacgcggatgggccgcgatgtcggtgagcgtgtggcgcggcgtttcgggcgctccattctggaacttggcgggaacaatgcgtccatcgtcaccccctccgccgatctggatctaacgctgcgggctgttgctttcggtgctatgggaaccgccggccagcgctgcacgaccctgcgtcgcctgttcgtgcattccagcgtctatgacacgctcgtgccaaagctggtcaatgtctacaagacgatttcggtcggtaacccggttgagggggacgcgctcgttggtccgctgatcgatgtggcgtccttcaaaatgatgcaggacagcctgaaagccgccgcggacgcaggtggcacggttcatggcggaacgcgtgtggatgtgaacggcgaggcgtcgttctatgtccgtccggcaatggtcgagatgccgtcgcagacggggccggtgctggaagaaaccttcgcgccgatcctttacgtcatgaaatatgacacgctcgatgaagccatcgcgcttcagaacgacgtggtgcagggcctgtcgtcttcgatctttgcgaccgatatccgcgaggtcgagcagttcctgtctgcgcagggttccgactgcggtattgcgaacgtcaatatggggccgtccggcgcagaaatcgggggagccttcgggggcgagaaggaaactggcggcgggcgggagtccggttccgatgcgtggaaagcctatatgcgccggcagaccaatgccatcaattacgggcggaccctgccgcttgcgcagggtgtgaagttcgatgtgtatggcggttcggcggacgcctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 112 - DNA, 6247 bp, Artificial
  plasmid, pCDFDuet-sceADH-goxALDH
taatacgactcactataggggaattgtgagcggataacaattcccctgtagaaataattttgtttaactttaataaggagatataccatggcgtcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgacggcatctctccgttgcgaagtggcagcgctcctcgacagccttggcgtgtcacctgatctgtatacggggggcagtctggcggtgaagtctccgctgacgggagaggtgattgccgaggtctcggaagtcagcgcggagcaggcgaaggagacgatcgccagttcactcgacgcgttcaaagcctggcgcagggttccggctccgcgtcgcggtgagcttgtccgcctgctgggtgaggagcttcgggccagcaaggaggcgctcggacggctcgtgacgctggaagtgggcaaggtgccgtccgaaggtctgggcgaagtgcaggaaatgatcgatatctgcgacttcgcggtcggtctgtcccgtcagctttatggcctgacgatccagtccgaacgtccggatcatcgcctgacagagcagtggcatcccgccgggccggtgggcatcatttcggcattcaatttccccgtggccgtctggtcctggaatgctgcgctggcactggtttgcggtgacagcgtgatctggaaaccttcggaaaagaccccgctgacagcgctggccacgcaggccctgttcctgaaagccgctgcccgtttcggcagtgacgcaccggtcgccctcagcacgcttctggtcggtggccgcgagatcggcgaactgatggtgaaagaccgtcgtgtccctgtcatctcggcgacgggttcgacgcggatgggccgcgatgtcggtgagcgtgtggcgcggcgtttcgggcgctccattctggaacttggcgggaacaatgcgtccatcgtcaccccctccgccgatctggatctaacgctgcgggctgttgctttcggtgctatgggaaccgccggccagcgctgcacgaccctgcgtcgcctgttcgtgcattccagcgtctatgacacgctcgtgccaaagctggtcaatgtctacaagacgatttcggtcggtaacccggttgagggggacgcgctcgttggtccgctgatcgatgtggcgtccttcaaaatgatgcaggacagcctgaaagccgccgcggacgcaggtggcacggttcatggcggaacgcgtgtggatgtgaacggcgaggcgtcgttctatgtccgtccggcaatggtcgagatgccgtcgcagacggggccggtgctggaagaaaccttcgcgccgatcctttacgtcatgaaatatgacacgctcgatgaagccatcgcgcttcagaacgacgtggtgcagggcctgtcgtcttcgatctttgcgaccgatatccgcgaggtcgagcagttcctgtctgcgcagggttccgactgcggtattgcgaacgtcaatatggggccgtccggcgcagaaatcgggggagccttcgggggcgagaaggaaactggcggcgggcgggagtccggttccgatgcgtggaaagcctatatgcgccggcagaccaatgccatcaattacgggcggaccctgccgcttgcgcagggtgtgaagttcgatgtgtatggcggttcggcggacgcctgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 113 - DNA, 2667 bp, Artificial
  fusion protein, His-sceADH::goxALDH
atgggcagcagccatcaccatcatcaccacagccaggatccgccttcgcaagtcattcctgaaaaacaaaaggctattgtcttttatgagacagatggaaaattggaatataaagacgtcacagttccggaacctaagcctaacgaaattttagtccacgttaaatattctggtgtttgtcatagtgacttgcacgcgtggcacggtgattggccatttcaattgaaatttccattaatcggtggtcacgaaggtgctggtgttgttgttaagttgggatctaacgttaagggctggaaagtcggtgattttgcaggtataaaatggttgaatgggacttgcatgtcctgtgaatattgtgaagtaggtaatgaatctcaatgtccttatttggatggtactggcttcacacatgatggtacttttcaagaatacgcaactgccgatgccgttcaagctgcccatattccaccaaacgtcaatcttgctgaagttgccccaatcttgtgtgcaggtatcactgtttataaggcgttgaaaagagccaatgtgataccaggccaatgggtcactatatccggtgcatgcggtggcttgggttctctggcaatccaatacgcccttgctatgggttacagggtcattggtatcgatggtggtaatgccaagcgaaagttatttgaacaattaggcggagaaatattcatcgatttcacggaagaaaaagacattgttggtgctataataaaggccactaatggcggttctcatggagttattaatgtgtctgtttctgaagcagctatcgaggcttctacgaggtattgtaggcccaatggtactgtcgtcctggttggtatgccagctcatgcttactgcaattccgatgttttcaatcaagttgtaaaatcaatctccatcgttggatcttgtgttggaaatagagctgatacaagggaggctttagatttcttcgccagaggtttgatcaaatctccgatccacttagctggcctatcggatgttcctgaaatttttgcaaagatggagaagggtgaaattgttggtagatatgttgttgagacttctaaaaagcttagcggcggcagcggcagcggcggcagcggccatatgacggcatctctccgttgcgaagtggcagcgctcctcgacagccttggcgtgtcacctgatctgtatacggggggcagtctggcggtgaagtctccgctgacgggagaggtgattgccgaggtctcggaagtcagcgcggagcaggcgaaggagacgatcgccagttcactcgacgcgttcaaagcctggcgcagggttccggctccgcgtcgcggtgagcttgtccgcctgctgggtgaggagcttcgggccagcaaggaggcgctcggacggctcgtgacgctggaagtgggcaaggtgccgtccgaaggtctgggcgaagtgcaggaaatgatcgatatctgcgacttcgcggtcggtctgtcccgtcagctttatggcctgacgatccagtccgaacgtccggatcatcgcctgacagagcagtggcatcccgccgggccggtgggcatcatttcggcattcaatttccccgtggccgtctggtcctggaatgctgcgctggcactggtttgcggtgacagcgtgatctggaaaccttcggaaaagaccccgctgacagcgctggccacgcaggccctgttcctgaaagccgctgcccgtttcggcagtgacgcaccggtcgccctcagcacgcttctggtcggtggccgcgagatcggcgaactgatggtgaaagaccgtcgtgtccctgtcatctcggcgacgggttcgacgcggatgggccgcgatgtcggtgagcgtgtggcgcggcgtttcgggcgctccattctggaacttggcgggaacaatgcgtccatcgtcaccccctccgccgatctggatctaacgctgcgggctgttgctttcggtgctatgggaaccgccggccagcgctgcacgaccctgcgtcgcctgttcgtgcattccagcgtctatgacacgctcgtgccaaagctggtcaatgtctacaagacgatttcggtcggtaacccggttgagggggacgcgctcgttggtccgctgatcgatgtggcgtccttcaaaatgatgcaggacagcctgaaagccgccgcggacgcaggtggcacggttcatggcggaacgcgtgtggatgtgaacggcgaggcgtcgttctatgtccgtccggcaatggtcgagatgccgtcgcagacggggccggtgctggaagaaaccttcgcgccgatcctttacgtcatgaaatatgacacgctcgatgaagccatcgcgcttcagaacgacgtggtgcagggcctgtcgtcttcgatctttgcgaccgatatccgcgaggtcgagcagttcctgtctgcgcagggttccgactgcggtattgcgaacgtcaatatggggccgtccggcgcagaaatcgggggagccttcgggggcgagaaggaaactggcggcgggcgggagtccggttccgatgcgtggaaagcctatatgcgccggcagaccaatgccatcaattacgggcggaccctgccgcttgcgcagggtgtgaagttcgatgtgtatggcggttcggcggacgcc

SEQ ID NO: 114 - protein, 889 aa, Artificial
  fusion protein, His-sceADH::goxALDH
MGSSHHHHHHSQDPPSQVIPEKQKAIVFYETDGKLEYKDVTVPEPKPNEILVHVKYSGVCHSDLHAWHGDWPFQLKFPLIGGHEGAGVVVKLGSNVKGWKVGDFAGIKWLNGTCMSCEYCEVGNESQCPYLDGTGFTHDGTFQEYATADAVQAAHIPPNVNLAEVAPILCAGITVYKALKRANVIPGQWVTISGACGGLGSLAIQYALAMGYRVIGIDGGNAKRKLFEQLGGEIFIDFTEEKDIVGAIIKATNGGSHGVINVSVSEAAIEASTRYCRPNGTVVLVGMPAHAYCNSDVFNQVVKSISIVGSCVGNRADTREALDFFARGLIKSPIHLAGLSDVPEIFAKMEKGEIVGRYVVETSKKLSGGSGSGGSGHMTASLRCEVAALLDSLGVSPDLYTGGSLAVKSPLTGEVIAEVSEVSAEQAKETIASSLDAFKAWRRVPAPRRGELVRLLGEELRASKEALGRLVTLEVGKVPSEGLGEVQEMIDICDFAVGLSRQLYGLTIQSERPDHRLTEQWHPAGPVGIISAFNFPVAVWSWNAALALVCGDSVIWKPSEKTPLTALATQALFLKAAARFGSDAPVALSTLLVGGREIGELMVKDRRVPVISATGSTRMGRDVGERVARRFGRSILELGGNNASIVTPSADLDLTLRAVAFGAMGTAGQRCTTLRRLFVHSSVYDTLVPKLVNVYKTISVGNPVEGDALVGPLIDVASFKMMQDSLKAAADAGGTVHGGTRVDVNGEASFYVRPAMVEMPSQTGPVLEETFAPILYVMKYDTLDEAIALQNDVVQGLSSSIFATDIREVEQFLSAQGSDCGIANVNMGPSGAEIGGAFGGEKETGGGRESGSDAWKAYMRRQTNAINYGRTLPLAQGVKFDVYGGSADA

SEQ ID NO: 115 - DNA, 42 bp, Artificial
  PCR Primer
aataatctcgagggcgtccgccgaaccgccatacacatcgaa

SEQ ID NO: 116 - DNA, 42 bp, Artificial
  PCR Primer
aataatcatatgcgcaaggcggcaggcaagtacgggaacacc

SEQ ID NO: 117 - DNA, 42 bp, Artificial
  PCR Primer
aataatctcgagttaaagccccagcacctccccccagggcgt

SEQ ID NO: 118 - DNA, 7933 bp, Artificial
  plasmid, pETDuet-pfuADH::tthALDH
taatacgactcactataggggaattgtgagcggataacaattcccctctagaaataattttgtttaactttaagaaggagatataccatgggcagcagccatcaccatcatcaccacagccaggatccgtttttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaaacttgatgaaatggtagagagagcctatgaagatcccctcgttaacttcaacccagttgagccgagaaaagaggacattagaagaatttatgagagggccttctatggagataagcttagcggcggcagcggcagcggcggcagcggccatatgcgcaaggcggcaggcaagtacgggaacaccttggagtttggccacctcgtgggcggggaggaggtcctggagggcccccttctggagcgccgcaacccctcggaccgggaggacgtggttgcccgctttcccgaggcggacaaggacctggtgcgcaaggccgccttgaaggcccgggaggccttcgccgagtggagccggacccccgcccccatccggggccaggtcctcttcaacctggtgaagatcctggagcgggagaagcccaccctcacccggctcatggtgcgggaggtgggcaagacccccaaggaggcggcgggggacgtgcaggaggccatagacaccgccctcttcttcgcctcggagggccgcaggctttacggccagaccgtccccagcgagatgcgggacaaggagcttttcaccttccgcaggcccttgggcgtggtggggatcatcaccgccgggaacttccccatcgccgtccccagctggaagctcatccccgccgtcctcacggggaacaccgtcgtgtggaagccttccgaggacgcccccaccctctccttcgtcttcgccaagctctttgaggaggcgggcctgcccccgggcgtcctcaacgtcgtcttcggcggcgggaagggctccacgggccagtggatggtggagctcatggacgagggcctcttccagaagttcgccttcaccggctccacccaggtggggcgctggatcggggaggtggcggggcggaacctgatcaggcccaccctggagcttgggggcaagaaccccttggtggtcatgcgggacgccgacctggacctcgccgtggagggggcctggtggagcgccttcgccacgggagggcagcggtgcacctccgcggggaacatcctggtggatgcccccatctacgaggagttcaagcgccgcttcctggagcgggtggaggccaccctggtgggcaaccccctcctccaccccgaggtcacctacgggcccttcatcaacgagcgcttcttcgcccgctggcaggagcactaccgggtgggggaggcggagggggcgaggctcctcttcggccggggccggatcacgcgggaaaacccctacccccgcttcctgggggacccggaggccgggctttacggctggcccacggtctgggaggtgaggccggggacgcgcctcttcacggaggaggtcttcgggcccacgatcaacctcgtcaaggtggacgggattgaggaggccatagcggtggccaacagcaccccctacggcctttctagcgccatctacaccaaccaccgccactgggcctacctcttcaaggtgggcatccgggccggcatgaccagcatcaacaacgccaccgtgggcgccgaggcccacctccccttcggcggggtgaaggcgagcggcaacggggggagggagtcggggatctgggtcttggaggagtacacctactggcacgccgtgaacgaggagtactcggggaggctgcagctcgcccagatggacacgggctacgtgagccccaaggcgcccacgccctggggggaggtgctggggctttaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 119 - DNA, 6373 bp, Artificial
  plasmid, pCDFDuet-pfuADH-tthALDH
taatacgactcactataggggaattgtgagcggataacaattcccctgtagaaataattttgtttaactttaataaggagatataccatggcgttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaaacttgatgaaatggtagagagagcctatgaagatcccctcgttaacttcaacccagttgagccgagaaaagaggacattagaagaatttatgagagggccttctatggagataagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcgcaaggcggcaggcaagtacgggaacaccttggagtttggccacctcgtgggcggggaggaggtcctggagggcccccttctggagcgccgcaacccctcggaccgggaggacgtggttgcccgctttcccgaggcggacaaggacctggtgcgcaaggccgccttgaaggcccgggaggccttcgccgagtggagccggacccccgcccccatccggggccaggtcctcttcaacctggtgaagatcctggagcgggagaagcccaccctcacccggctcatggtgcgggaggtgggcaagacccccaaggaggcggcgggggacgtgcaggaggccatagacaccgccctcttcttcgcctcggagggccgcaggctttacggccagaccgtccccagcgagatgcgggacaaggagcttttcaccttccgcaggcccttgggcgtggtggggatcatcaccgccgggaacttccccatcgccgtccccagctggaagctcatccccgccgtcctcacggggaacaccgtcgtgtggaagccttccgaggacgcccccaccctctccttcgtcttcgccaagctctttgaggaggcgggcctgcccccgggcgtcctcaacgtcgtcttcggcggcgggaagggctccacgggccagtggatggtggagctcatggacgagggcctcttccagaagttcgccttcaccggctccacccaggtggggcgctggatcggggaggtggcggggcggaacctgatcaggcccaccctggagcttgggggcaagaaccccttggtggtcatgcgggacgccgacctggacctcgccgtggagggggcctggtggagcgccttcgccacgggagggcagcggtgcacctccgcggggaacatcctggtggatgcccccatctacgaggagttcaagcgccgcttcctggagcgggtggaggccaccctggtgggcaaccccctcctccaccccgaggtcacctacgggcccttcatcaacgagcgcttcttcgcccgctggcaggagcactaccgggtgggggaggcggagggggcgaggctcctcttcggccggggccggatcacgcgggaaaacccctacccccgcttcctgggggacccggaggccgggctttacggctggcccacggtctgggaggtgaggccggggacgcgcctcttcacggaggaggtcttcgggcccacgatcaacctcgtcaaggtggacgggattgaggaggccatagcggtggccaacagcaccccctacggcctttctagcgccatctacaccaaccaccgccactgggcctacctcttcaaggtgggcatccgggccggcatgaccagcatcaacaacgccaccgtgggcgccgaggcccacctccccttcggcggggtgaaggcgagcggcaacggggggagggagtcggggatctgggtcttggaggagtacacctactggcacgccgtgaacgaggagtactcggggaggctgcagctcgcccagatggacacgggctacgtgagccccaaggcgcccacgccctggggggaggtgctggggctttaactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 120 - DNA, 2793 bp, Artificial
  fusion protein, His-pfuADH::tthALDH
atgggcagcagccatcaccatcatcaccacagccaggatccgtttttcctaaagactaagattatcgagggaaggggaagtttggagtatcttcaaaaggaagctattgggttcgagagggctttaatcttagcgtcaagctctatgaaaaggttagggtttctgaaggaagctgaagactatctaagggaaggtggagttgaggtaatttcaatagcaggccttcctcctgagcccacgaaagacgttgtcgacgagttcctcccaaaagttagggagttttctccagagatcctcattgcaattggtggaggaagtgttatcgatatggccaaagctctaaaggtattttacgatgtcccaggaatttcatttgaggaagttgcaatcaagagtagattttcgaagccgagacccgttccaaagctgaaaactccccttatagctattccctcaacgagcggtgctggaagtgaggtttcagcagcgagcgtgattaaggtaggggacgttaagtacacgattgtaactcccgagatagctcctgaagtggctatacttgaccctagattacccgaaggaatgcccccagaagttgccaggaattctggaatggatgttttagttcatgcgatagaggcttatgtttcaaaggcttcctcaccctttacagatgcaatggcagttaaagcggcaaaagctgtgttggagcatttagaggagagcattagaggagacaaaaatgccagagagaaaatccactatgcagctacaatggctggcatagctttcttaaacgcaaggcttggactcgttcatgcgatgagtcataaggccgcatggataggaccgcatggtctcgtaaatgccattctacttccctatgtgatggagtataacatggcacgcaggagagagaaatatgatgcattggcgagggaattgggatttgacaatgctgaagagctccttgaagagataaaagctctgaacgaaaggcttggaataggacccatctcaagttacgtcagcgaggaggagttcttagcgaaacttgatgaaatggtagagagagcctatgaagatcccctcgttaacttcaacccagttgagccgagaaaagaggacattagaagaatttatgagagggccttctatggagataagcttagcggcggcagcggcagcggcggcagcggccatatgcgcaaggcggcaggcaagtacgggaacaccttggagtttggccacctcgtgggcggggaggaggtcctggagggcccccttctggagcgccgcaacccctcggaccgggaggacgtggttgcccgctttcccgaggcggacaaggacctggtgcgcaaggccgccttgaaggcccgggaggccttcgccgagtggagccggacccccgcccccatccggggccaggtcctcttcaacctggtgaagatcctggagcgggagaagcccaccctcacccggctcatggtgcgggaggtgggcaagacccccaaggaggcggcgggggacgtgcaggaggccatagacaccgccctcttcttcgcctcggagggccgcaggctttacggccagaccgtccccagcgagatgcgggacaaggagcttttcaccttccgcaggcccttgggcgtggtggggatcatcaccgccgggaacttccccatcgccgtccccagctggaagctcatccccgccgtcctcacggggaacaccgtcgtgtggaagccttccgaggacgcccccaccctctccttcgtcttcgccaagctctttgaggaggcgggcctgcccccgggcgtcctcaacgtcgtcttcggcggcgggaagggctccacgggccagtggatggtggagctcatggacgagggcctcttccagaagttcgccttcaccggctccacccaggtggggcgctggatcggggaggtggcggggcggaacctgatcaggcccaccctggagcttgggggcaagaaccccttggtggtcatgcgggacgccgacctggacctcgccgtggagggggcctggtggagcgccttcgccacgggagggcagcggtgcacctccgcggggaacatcctggtggatgcccccatctacgaggagttcaagcgccgcttcctggagcgggtggaggccaccctggtgggcaaccccctcctccaccccgaggtcacctacgggcccttcatcaacgagcgcttcttcgcccgctggcaggagcactaccgggtgggggaggcggagggggcgaggctcctcttcggccggggccggatcacgcgggaaaacccctacccccgcttcctgggggacccggaggccgggctttacggctggcccacggtctgggaggtgaggccggggacgcgcctcttcacggaggaggtcttcgggcccacgatcaacctcgtcaaggtggacgggattgaggaggccatagcggtggccaacagcaccccctacggcctttctagcgccatctacaccaaccaccgccactgggcctacctcttcaaggtgggcatccgggccggcatgaccagcatcaacaacgccaccgtgggcgccgaggcccacctccccttcggcggggtgaaggcgagcggcaacggggggagggagtcggggatctgggtcttggaggagtacacctactggcacgccgtgaacgaggagtactcggggaggctgcagctcgcccagatggacacgggctacgtgagccccaaggcgcccacgccctggggggaggtgctggggctt

SEQ ID NO: 121 - protein, 931 aa, Artificial
  fusion protein, His-pfuADH::tthALDH
MGSSHHHHHHSQDPFFLKTKIIEGRGSLEYLQKEAIGFERALILASSSMKRLGFLKEAEDYLREGGVEVISIAGLPPEPTKDVVDEFLPKVREFSPEILIAIGGGSVIDMAKALKVFYDVPGISFEEVAIKSRFSKPRPVPKLKTPLIAIPSTSGAGSEVSAASVIKVGDVKYTIVTPEIAPEVAILDPRLPEGMPPEVARNSGMDVLVHAIEAYVSKASSPFTDAMAVKAAKAVLEHLEESIRGDKNAREKIHYAATMAGIAFLNARLGLVHAMSHKAAWIGPHGLVNAILLPYVMEYNMARRREKYDALARELGFDNAEELLEEIKALNERLGIGPISSYVSEEEFLAKLDEMVERAYEDPLVNFNPVEPRKEDIRRIYERAFYGDKLSGGSGSGGSGHMRKAAGKYGNTLEFGHLVGGEEVLEGPLLERRNPSDREDVVARFPEADKDLVRKAALKAREAFAEWSRTPAPIRGQVLFNLVKILEREKPTLTRLMVREVGKTPKEAAGDVQEAIDTALFFASEGRRLYGQTVPSEMRDKELFTFRRPLGVVGIITAGNFPIAVPSWKLIPAVLTGNTVVWKPSEDAPTLSFVFAKLFEEAGLPPGVLNVVFGGGKGSTGQWMVELMDEGLFQKFAFTGSTQVGRWIGEVAGRNLIRPTLELGGKNPLVVMRDADLDLAVEGAWWSAFATGGQRCTSAGNILVDAPIYEEFKRRFLERVEATLVGNPLLHPEVTYGPFINERFFARWQEHYRVGEAEGARLLFGRGRITRENPYPRFLGDPEAGLYGWPTVWEVRPGTRLFTEEVFGPTINLVKVDGIEEAIAVANSTPYGLSSAIYTNHRHWAYLFKVGIRAGMTSINNATVGAEAHLPFGGVKASGNGGRESGIWVLEEYTYWHAVNEEYSGRLQLAQMDTGYVSPKAPTPWGEVLGL

SEQ ID NO: 122 - DNA, 42 bp, Artificial
  PCR Primer
aataatctcgagaagccccagcacctccccccagggcgtggg

SEQ ID NO: 123 - DNA, 42 bp, Artificial
  PCR Primer
aataatcatatgcaagcctattttgaccagctcgatcgcgtt

SEQ ID NO: 124 - DNA, 42 bp, Artificial
  PCR Primer
aataatctcgagttatttgtcgaacagataatggtttaccag

SEQ ID NO: 125 - DNA, 7321 bp, Artificial
  plasmid, pETDuet-busGDH::ecoISO
ggggaattgtgagcggataacaattcccctctagaaataattttgtttaactttaagaaggagatataccatgggcagcagccatcaccatcatcaccacagccaggatccgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttagcggcggcagcggcagcggcggcagcggccatatgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaattaatacgactcactata

SEQ ID NO: 126 - DNA, 5761 bp, Artificial
  plasmid, pCDFDuet-busGDH-ecoISO
taatacgactcactataggggaattgtgagcggataacaattcccctgtagaaataattttgtttaactttaataaggagatataccatggcgccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaataactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 127 - DNA, 2181 bp, Artificial
  fusion protein, His-busGDH::ecoISO
atgggcagcagccatcaccatcatcaccacagccaggatccgtatccggatttaaaaggaaaagtcgtcgctattacaggagctgcttcagggctcggaaaggcgatggccattcgcttcggcaaggagcaggcaaaagtggttatcaactattatagtaataaacaagatccgaacgaggtaaaagaagaggtcatcaaggcgggcggtgaagctgttgtcgtccaaggagatgtcacgaaagaggaagatgtaaaaaatatcgtgcaaacggcaattaaggagttcggcacactcgatattatgattaataatgccggtcttgaaaatcctgtgccatctcacgaaatgccgctcaaggattgggataaagtcatcggcacgaacttaacgggtgcctttttaggaagccgtgaagcgattaaatatttcgtagaaaacgatatcaagggaaatgtcattaacatgtccagtgtgcacgaagtgattccttggccgttatttgtccactatgcggcaagtaaaggcgggataaagctgatgacagaaacattagcgttggaatacgcgccgaagggcattcgcgtcaataatattgggccaggtgcgatcaacacgccaatcaatgctgaaaaattcgctgaccctaaacagaaagctgatgtagaaagcatgattccaatgggatatatcggcgaaccggaggagatcgccgcagtagcagcctggcttgcttcgaaggaagccagctacgtcacaggcatcacgttattcgcggacggcggtatgacacaatatccttcattccaggcaggccgcggtaagcttagcggcggcagcggcagcggcggcagcggccatatgcaagcctattttgaccagctcgatcgcgttcgttatgaaggctcaaaatcctcaaacccgttagcattccgtcactacaatcccgacgaactggtgttgggtaagcgtatggaagagcacttgcgttttgccgcctgctactggcacaccttctgctggaacggggcggatatgtttggtgtgggggcgtttaatcgtccgtggcagcagcctggtgaggcactggcgttggcgaagcgtaaagcagatgtcgcatttgagtttttccacaagttacatgtgccattttattgcttccacgatgtggatgtttcccctgagggcgcgtcgttaaaagagtacatcaataattttgcgcaaatggttgatgtcctggcaggcaagcaagaagagagcggcgtgaagctgctgtggggaacggccaactgctttacaaaccctcgctacggcgcgggtgcggcgacgaacccagatcctgaagtcttcagctgggcggcaacgcaagttgttacagcgatggaagcaacccataaattgggcggtgaaaactatgtcctgtggggcggtcgtgaaggttacgaaacgctgttaaataccgacttgcgtcaggagcgtgaacaactgggccgctttatgcagatggtggttgagcataaacataaaatcggtttccagggcacgttgcttatcgaaccgaaaccgcaagaaccgaccaaacatcaatatgattacgatgccgcgacggtctatggcttcctgaaacagtttggtctggaaaaagagattaaactgaacattgaagctaaccacgcgacgctggcaggtcactctttccatcatgaaatagccaccgccattgcgcttggcctgttcggttctgtcgacgccaaccgtggcgatgcgcaactgggctgggacaccgaccagttcccgaacagtgtggaagagaatgcgctggtgatgtatgaaattctcaaagcaggcggtttcaccaccggtggtctgaacttcgatgccaaagtacgtcgtcaaagtactgataaatatgatctgttttacggtcatatcggcgcgatggatacgatggcactggcgctgaaaattgcagcgcgcatgattgaagatggcgagctggataaacgcatcgcgcagcgttattccggctggaatagcgaattgggccagcaaatcctgaaaggccaaatgtcactggcagatttagccaaatatgctcaggaacatcatttgtctccggtgcatcagagtggtcgccaggaacaactggaaaatctggtaaaccattatctgttcgacaaa

SEQ ID NO: 128 - protein, 727 aa, Artificial
  fusion protein, His-busGDH::ecoISO
MGSSHHHHHHSQDPYPDLKGKVVAITGAASGLGKAMAIRFGKEQAKVVINYYSNKQDPNEVKEEVIKAGGEAVVVQGDVTKEEDVKNIVQTAIKEFGTLDIMINNAGLENPVPSHEMPLKDWDKVIGTNLTGAFLGSREAIKYFVENDIKGNVINMSSVHEVIPWPLFVHYAASKGGIKLMTETLALEYAPKGIRVNNIGPGAINTPINAEKFADPKQKADVESMIPMGYIGEPEEIAAVAAWLASKEASYVTGITLFADGGMTQYPSFQAGRGKLSGGSGSGGSGHMQAYFDQLDRVRYEGSKSSNPLAFRHYNPDELVLGKRMEEHLRFAACYWHTFCWNGADMFGVGAFNRPWQQPGEALALAKRKADVAFEFFHKLHVPFYCFHDVDVSPEGASLKEYINNFAQMVDVLAGKQEESGVKLLWGTANCFTNPRYGAGAATNPDPEVFSWAATQVVTAMEATHKLGGENYVLWGGREGYETLLNTDLRQEREQLGRFMQMVVEHKHKIGFQGTLLIEPKPQEPTKHQYDYDAATVYGFLKQFGLEKEIKLNIEANHATLAGHSFHHEIATAIALGLFGSVDANRGDAQLGWDTDQFPNSVEENALVMYEILKAGGFTTGGLNFDAKVRRQSTDKYDLFYGHIGAMDTMALALKIAARMIEDGELDKRIAQRYSGWNSELGQQILKGQMSLADLAKYAQEHHLSPVHQSGRQEQLENLVNHYLFDK

SEQ ID NO: 129 - DNA, 42 bp, Artificial
  PCR Primer
aataatctcgagtttgtcgaacagataatggtttaccagatt

SEQ ID NO: 130 - DNA, 42 bp, Artificial
  PCR Primer
aataatcatatggcagaatttttcccagaaatcccaaagatt

SEQ ID NO: 131 - DNA, 42 bp, Artificial
  PCR Primer
aataatctcgagtcacctcagttctgctattgtcttcactat

SEQ ID NO: 132 - DNA, 7327 bp, Artificial
  plasmid, pETDuet-pfuGDH::tmaISO
taatacgactcactataggggaattgtgagcggataacaattcccctctagaaataattttgtttaactttaagaaggagatataccatgggcagcagccatcaccatcatcaccacagccaggatccgaacattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttagcggcggcagcggcagcggcggcagcggccatatggcagaatttttcccagaaatcccaaagattcagtttgaaggtaaggaaagcacgaacccgctggcgttcaggttctacgatccgaacgaggtgatcgatgggaagcccctcaaggatcatctgaagttttccgtcgccttctggcacactttcgtgaacgagggaagagatccgtttggagatccaaccgctgaacgcccatggaacaggttctccgatcccatggacaaagcgtttgcaagggtggacgctctcttcgagttctgcgaaaagctcaacatcgagtacttctgtttccacgacagagacatcgctcccgaaggaaagacgctcagagagacaaacaagatactggacaaagtggtggaaagaattaaggaaagaatgaaggacagcaacgtgaaactcctctggggaacggcgaatctgttctcccacccgaggtacatgcacggcgccgcgacgacatgcagtgcggatgtctttgcctacgcggcggcccaggtgaagaaggccctggagatcacaaaagaactaggaggagaaggctacgtcttctggggtggaagagaaggctatgaaacactcctcaacacagatctcgggctggaactcgaaaatctcgcgagatttctcagaatggcagtggagtacgcaaagaagatcggcttcaccggacagttcctcatagaacccaagccgaaagaacccaccaagcaccagtacgatttcgatgtggcaacggcctacgctttcctgaagaaccacggccttgatgagtacttcaagttcaacattgaagcgaaccacgcgacacttgccggtcacacattccagcacgaactgaggatggcaaggatcctcggaaaacttggaagcatcgacgccaaccagggagatctcctgctcggctgggacaccgaccagttcccaacgaacatctacgacacaacacttgccatgtacgaagtgataaaagcaggaggattcacaaagggtggcctcaacttcgacgcgaaggtgaggagggcttcctacaaggtagaagatctcttcatcggtcacatcgcaggaatggacaccttcgccctcggcttcaagatagcgtacaaactcgcgaaagacggagtgttcgacaagttcatcgaggaaaaataccgtagcttcaaagaaggtatcggaaaagagatcgtagaaggaaaaaccgatttcgaaaaactcgaagagtatataatagacaaagaagatatcgaacttccatctggaaagcaggagtacctcgaaagcctgctcaacagctacatagtgaagacaatagcagaactgaggtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaaggaggaactatatccggattggcgaatgggacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttctggcggcacgatggcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatcatgattgaagcatttatcagggttattgtctcatgagcggatacatatttgaatgtatttagaaaaataaacaaataggtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcctgatgcggtattttctccttacgcatctgtgcggtatttcacaccgcatatatggtgcactctcagtacaatctgctctgatgccgcatagttaagccagtatacactccgctatcgctacgtgactgggtcatggctgcgccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgaggcagctgcggtaaagctcatcagcgtggtcgtgaagcgattcacagatgtctgcctgttcatccgcgtccagctcgttgagtttctccagaagcgttaatgtctggcttctgataaagcgggccatgttaagggcggttttttcctgtttggtcactgatgcctccgtgtaagggggatttctgttcatgggggtaatgataccgatgaaacgagagaggatgctcacgatacgggttactgatgatgaacatgcccggttactggaacgttgtgagggtaaacaactggcggtatggatgcggcgggaccagagaaaaatcactcagggtcaatgccagcgcttcgttaatacagatgtaggtgttccacagggtagccagcagcatcctgcgatgcagatccggaacataatggtgcagggcgctgacttccgcgtttccagactttacgaaacacggaaaccgaagaccattcatgttgttgctcaggtcgcagacgttttgcagcagcagtcgcttcacgttcgctcgcgtatcggtgattcattctgctaaccagtaaggcaaccccgccagcctagccgggtcctcaacgacaggagcacgatcatgctagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgatgccggccacgatgcgtccggcgtagaggatcgagatcgatctcgatcccgcgaaat

SEQ ID NO: 133 - DNA, 5767 bp, Artificial
  plasmid, pCDFDuet-pfuGDH-tmaISO
taatacgactcactataggggaattgtgagcggataacaattcccctgtagaaataattttgtttaactttaataaggagatataccatggcgattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttgcggccgcataatgcttaagtcgaacagaaagtaatcgtattgtacacggccgcataatcgaaattaatacgactcactataggggaattgtgagcggataacaattccccatcttagtatattagttaagtataagaaggagatatacatatggcagaatttttcccagaaatcccaaagattcagtttgaaggtaaggaaagcacgaacccgctggcgttcaggttctacgatccgaacgaggtgatcgatgggaagcccctcaaggatcatctgaagttttccgtcgccttctggcacactttcgtgaacgagggaagagatccgtttggagatccaaccgctgaacgcccatggaacaggttctccgatcccatggacaaagcgtttgcaagggtggacgctctcttcgagttctgcgaaaagctcaacatcgagtacttctgtttccacgacagagacatcgctcccgaaggaaagacgctcagagagacaaacaagatactggacaaagtggtggaaagaattaaggaaagaatgaaggacagcaacgtgaaactcctctggggaacggcgaatctgttctcccacccgaggtacatgcacggcgccgcgacgacatgcagtgcggatgtctttgcctacgcggcggcccaggtgaagaaggccctggagatcacaaaagaactaggaggagaaggctacgtcttctggggtggaagagaaggctatgaaacactcctcaacacagatctcgggctggaactcgaaaatctcgcgagatttctcagaatggcagtggagtacgcaaagaagatcggcttcaccggacagttcctcatagaacccaagccgaaagaacccaccaagcaccagtacgatttcgatgtggcaacggcctacgctttcctgaagaaccacggccttgatgagtacttcaagttcaacattgaagcgaaccacgcgacacttgccggtcacacattccagcacgaactgaggatggcaaggatcctcggaaaacttggaagcatcgacgccaaccagggagatctcctgctcggctgggacaccgaccagttcccaacgaacatctacgacacaacacttgccatgtacgaagtgataaaagcaggaggattcacaaagggtggcctcaacttcgacgcgaaggtgaggagggcttcctacaaggtagaagatctcttcatcggtcacatcgcaggaatggacaccttcgccctcggcttcaagatagcgtacaaactcgcgaaagacggagtgttcgacaagttcatcgaggaaaaataccgtagcttcaaagaaggtatcggaaaagagatcgtagaaggaaaaaccgatttcgaaaaactcgaagagtatataatagacaaagaagatatcgaacttccatctggaaagcaggagtacctcgaaagcctgctcaacagctacatagtgaagacaatagcagaactgaggtgactcgagtctggtaaagaaaccgctgctgcgaaatttgaacgccagcacatggactcgtctactagcgcagcttaattaacctaggctgctgccaccgctgagcaataactagcataaccccttggggcctctaaacgggtcttgaggggttttttgctgaaacctcaggcatttgagaagcacacggtcacactgcttccggtagtcaataaaccggtaaaccagcaatagacataagcggctatttaacgaccctgccctgaaccgacgaccgggtcatcgtggccggatcttgcggcccctcggcttgaacgaattgttagacattatttgccgactaccttggtgatctcgcctttcacgtagtggacaaattcttccaactgatctgcgcgcgaggccaagcgatcttcttcttgtccaagataagcctgtctagcttcaagtatgacgggctgatactgggccggcaggcgctccattgcccagtcggcagcgacatccttcggcgcgattttgccggttactgcgctgtaccaaatgcgggacaacgtaagcactacatttcgctcatcgccagcccagtcgggcggcgagttccatagcgttaaggtttcatttagcgcctcaaatagatcctgttcaggaaccggatcaaagagttcctccgccgctggacctaccaaggcaacgctatgttctcttgcttttgtcagcaagatagccagatcaatgtcgatcgtggctggctcgaagatacctgcaagaatgtcattgcgctgccattctccaaattgcagttcgcgcttagctggataacgccacggaatgatgtcgtcgtgcacaacaatggtgacttctacagcgcggagaatctcgctctctccaggggaagccgaagtttccaaaaggtcgttgatcaaagctcgccgcgttgtttcatcaagccttacggtcaccgtaaccagcaaatcaatatcactgtgtggcttcaggccgccatccactgcggagccgtacaaatgtacggccagcaacgtcggttcgagatggcgctcgatgacgccaactacctctgatagttgagtcgatacttcggcgatcaccgcttccctcatactcttcctttttcaatattattgaagcatttatcagggttattgtctcatgagcggatacatatttgaatgtatttagaaaaataaacaaatagctagctcactcggtcgctacgctccgggcgtgagactgcggcgggcgctgcggacacatacaaagttacccacagattccgtggataagcaggggactaacatgtgaggcaaaacagcagggccgcgccggtggcgtttttccataggctccgccctcctgccagagttcacataaacagacgcttttccggtgcatctgtgggagccgtgaggctcaaccatgaatctgacagtacgggcgaaacccgacaggacttaaagatccccaccgtttccggcgggtcgctccctcttgcgctctcctgttccgaccctgccgtttaccggatacctgttccgcctttctcccttacgggaagtgtggcgctttctcatagctcacacactggtatctcggctcggtgtaggtcgttcgctccaagctgggctgtaagcaagaactccccgttcagcccgactgctgcgccttatccggtaactgttcacttgagtccaacccggaaaagcacggtaaaacgccactggcagcagccattggtaactgggagttcgcagaggatttgtttagctaaacacgcggttgctcttgaagtgtgcgccaaagtccggctacactggaaggacagatttggttgctgtgctctgcgaaagccagttaccacggttaagcagttccccaactgacttaaccttcgatcaaaccacctccccaggtggttttttcgtttacagggcaaaagattacgcgcagaaaaaaaggatctcaagaagatcctttgatcttttctactgaaccgctctagatttcagtgcaatttatctcttcaaatgtagcacctgaagtcagccccatacgatataagttgtaattctcatgttagtcatgccccgcgcccaccggaaggagctgactgggttgaaggctctcaagggcatcggtcgagatcccggtgcctaatgagtgagctaacttacattaattgcgttgcgctcactgcccgctttccagtcgggaaacctgtcgtgccagctgcattaatgaatcggccaacgcgcggggagaggcggtttgcgtattgggcgccagggtggtttttcttttcaccagtgagacgggcaacagctgattgcccttcaccgcctggccctgagagagttgcagcaagcggtccacgctggtttgccccagcaggcgaaaatcctgtttgatggtggttaacggcgggatataacatgagctgtcttcggtatcgtcgtatcccactaccgagatgtccgcaccaacgcgcagcccggactcggtaatggcgcgcattgcgcccagcgccatctgatcgttggcaaccagcatcgcagtgggaacgatgccctcattcagcatttgcatggtttgttgaaaaccggacatggcactccagtcgccttcccgttccgctatcggctgaatttgattgcgagtgagatatttatgccagccagccagacgcagacgcgccgagacagaacttaatgggcccgctaacagcgcgatttgctggtgacccaatgcgaccagatgctccacgcccagtcgcgtaccgtcttcatgggagaaaataatactgttgatgggtgtctggtcagagacatcaagaaataacgccggaacattagtgcaggcagcttccacagcaatggcatcctggtcatccagcggatagttaatgatcagcccactgacgcgttgcgcgagaagattgtgcaccgccgctttacaggcttcgacgccgcttcgttctaccatcgacaccaccacgctggcacccagttgatcggcgcgagatttaatcgccgcgacaatttgcgacggcgcgtgcagggccagactggaggtggcaacgccaatcagcaacgactgtttgcccgccagttgttgtgccacgcggttgggaatgtaattcagctccgccatcgccgcttccactttttcccgcgttttcgcagaaacgtggctggcctggttcaccacgcgggaaacggtctgataagagacaccggcatactctgcgacatcgtataacgttactggtttcacattcaccaccctgaattgactctcttccgggcgctatcatgccataccgcgaaaggttttgcgccattcgatggtgtccgggatctcgacgctctcccttatgcgactcctgcattaggaaat

SEQ ID NO: 134 - DNA, 2187 bp, Artificial
  fusion protein, His-pfuGDH::tmaISO
atgggcagcagccatcaccatcatcaccacagccaggatccgaacattttaataacagcttcttcaagaggaataggcttcaatgtggccaaggaactcctcaaaaaaggacacagagtagtaatatcgtctagcaatgaaaaaaatttagaaaaagccctagaagagctaaaagaatatgaaaaagtctatccgattaaagcagatctctcaaaaaaagaagaaattaaaaacatggtagaaaaggcctgggaaattttaggaaaaatagatgtccttgtttggaatgcccctaatgtaaagtgtgaaccctgcatgattcacgaattagattatgaagattgggtagaaggagccttgcttcacataattgccccaggctacctaacaactcttctccttaataaatggttgaaaaataacacaagagggataataatctacttaagttctgcttcagttttagagccaatgcctcccctccttctcgcagacagtactagggcaggattaattcagcttgctaaaggaatatctagggcctatggaaagagaggaataagggcatacacagtactacttgggagctttgataccccaggagctagagaaaacctaagaagactagcagaaaaaaggggtgttgatccagaagagctatggaaaaaggaagttattgaaagaactcctcttcaaagaactggaaaatgggaagaactaggagaattaatagaattcttacttagcgataacgccgaatatatgctaggctctacaattctctttgatggagcaatgactaggagcgttcttcttaagcttagcggcggcagcggcagcggcggcagcggccatatggcagaatttttcccagaaatcccaaagattcagtttgaaggtaaggaaagcacgaacccgctggcgttcaggttctacgatccgaacgaggtgatcgatgggaagcccctcaaggatcatctgaagttttccgtcgccttctggcacactttcgtgaacgagggaagagatccgtttggagatccaaccgctgaacgcccatggaacaggttctccgatcccatggacaaagcgtttgcaagggtggacgctctcttcgagttctgcgaaaagctcaacatcgagtacttctgtttccacgacagagacatcgctcccgaaggaaagacgctcagagagacaaacaagatactggacaaagtggtggaaagaattaaggaaagaatgaaggacagcaacgtgaaactcctctggggaacggcgaatctgttctcccacccgaggtacatgcacggcgccgcgacgacatgcagtgcggatgtctttgcctacgcggcggcccaggtgaagaaggccctggagatcacaaaagaactaggaggagaaggctacgtcttctggggtggaagagaaggctatgaaacactcctcaacacagatctcgggctggaactcgaaaatctcgcgagatttctcagaatggcagtggagtacgcaaagaagatcggcttcaccggacagttcctcatagaacccaagccgaaagaacccaccaagcaccagtacgatttcgatgtggcaacggcctacgctttcctgaagaaccacggccttgatgagtacttcaagttcaacattgaagcgaaccacgcgacacttgccggtcacacattccagcacgaactgaggatggcaaggatcctcggaaaacttggaagcatcgacgccaaccagggagatctcctgctcggctgggacaccgaccagttcccaacgaacatctacgacacaacacttgccatgtacgaagtgataaaagcaggaggattcacaaagggtggcctcaacttcgacgcgaaggtgaggagggcttcctacaaggtagaagatctcttcatcggtcacatcgcaggaatggacaccttcgccctcggcttcaagatagcgtacaaactcgcgaaagacggagtgttcgacaagttcatcgaggaaaaataccgtagcttcaaagaaggtatcggaaaagagatcgtagaaggaaaaaccgatttcgaaaaactcgaagagtatataatagacaaagaagatatcgaacttccatctggaaagcaggagtacctcgaaagcctgctcaacagctacatagtgaagacaatagcagaactgagg

SEQ ID NO: 135 - protein, 729 aa, Artificial
  fusion protein, His-pfuGDH::tmaISO
MGSSHHHHHHSQDPNILITASSRGIGFNVAKELLKKGHRVVISSSNEKNLEKALEELKEYEKVYPIKADLSKKEEIKNMVEKAWEILGKIDVLVWNAPNVKCEPCMIHELDYEDWVEGALLHIIAPGYLTTLLLNKWLKNNTRGIIIYLSSASVLEPMPPLLLADSTRAGLIQLAKGISRAYGKRGIRAYTVLLGSFDTPGARENLRRLAEKRGVDPEELWKKEVIERTPLQRTGKWEELGELIEFLLSDNAEYMLGSTILFDGAMTRSVLLKLSGGSGSGGSGHMAEFFPEIPKIQFEGKESTNPLAFRFYDPNEVIDGKPLKDHLKFSVAFWHTFVNEGRDPFGDPTAERPWNRFSDPMDKAFARVDALFEFCEKLNIEYFCFHDRDIAPEGKTLRETNKILDKVVERIKERMKDSNVKLLWGTANLFSHPRYMHGAATTCSADVFAYAAAQVKKALEITKELGGEGYVFWGGREGYETLLNTDLGLELENLARFLRMAVEYAKKIGFTGQFLIEPKPKEPTKHQYDFDVATAYAFLKNHGLDEYFKFNIEANHATLAGHTFQHELRMARILGKLGSIDANQGDLLLGWDTDQFPTNIYDTTLAMYEVIKAGGFTKGGLNFDAKVRRASYKVEDLFIGHIAGMDTFALGFKIAYKLAKDGVFDKFIEEKYRSFKEGIGKEIVEGKTDFEKLEEYIIDKEDIELPSGKQEYLESLLNSYIVKTIAELR

SEQ ID NO: 136 - DNA, 42 bp, Artificial
  PCR Primer
aataatctcgagcctcagttctgctattgtcttcactatgta

SEQ ID NO: 137 - protein, 5 aa, Artificial
  spacer sequence
GGGGS

SEQ ID NO: 138 - protein, 8 aa, Artificial
  spacer sequence
STGDKQNA

SEQ ID NO: 139 - protein, 5 aa, Artificial
  spacer sequence
GSGSG

SEQ ID NO: 140 - protein, 5 aa, Artificial
  spacer sequence
SGGSG

SEQ ID NO: 141 - protein, 5 aa, Artificial
  spacer sequence
NGGGG

SEQ ID NO: 142 - protein, 14 aa, Artificial
  spacer sequence
EGKSSGSGSESKST

SEQ ID NO: 143 - protein, 5 aa, Artificial
  spacer sequence
SKSTS

SEQ ID NO: 144 - protein, 19 aa, Artificial
  spacer sequence
PVPSTPPTPSPSTPPTPSM

SEQ ID NO: 145 - protein, 8 aa, Artificial
  spacer sequence
GGGSGGGS

SEQ ID NO: 146 - protein, 10 aa, Artificial
  spacer sequence
SGGSGSGGSG

(19) United States
(12) Patent Application Publication
(10) Pub. No.: US 2007/0131546 A1
(43) Pub. Date: Jun. 14, 2007

(54) Enzyme electrode

(75) Inventors: Tsuyoshi Nomoto, Tokyo (JP); Wataru Kubo, Tokyo (JP); Tetsuya Yano, Tokyo (JP)

Correspondence Address: FITZPATRICK CELLA HARPER & SCINTO, 30 ROCKEFELLER PLAZA, NEW YORK, NY, 10112, US

(73) Assignee: CANON KABUSHIKI KAISHA, Tokyo (JP)

(21) Appl. No.: 11/634,921

(22) Filed: Dec. 7, 2006

(30) Foreign Application Priority Data
Dec. 9, 2005 (JP) 2005-356930 (PAT.

(51) Int. Cl. G01N 33/487; H01M 4/90

(52) U.S. Cl. 204/403.01; 429/43

(57) ABSTRACT

There is provided with an enzyme electrode which can be used as a sensor with high sensitivity, a biofuel cell with high output, and an electrochemical reaction device with high reaction efficiency. The enzyme electrode has a conductive base plate, a fusion protein immobilized to the conductive base plate and an electron transfer mediator, wherein the fusion protein is a fusion protein of a enzyme 1 to catalyze a chemical reaction for producing a reaction product 1 from a reaction substrate 1 and a enzyme 2 to catalyze a chemical reaction for producing a reaction product 2 from a reaction substrate 2, and at least one chemical substance of the reaction product 1 is identical to at least one chemical substance of the reaction substrate 2.

### BACKGROUND OF THE INVENTION

1. Field of the Invention

The present invention relates to an enzyme electrode, which can be used as an electrode of a biosensor, a biofuel cell or an electrochemical reaction device.

2. Description of the Related Art

An enzyme, which is a protein biocatalyst created in a living cell, strongly acts under a condition milder than that of a normal catalyst. Also, a substrate which is a substance for causing a chemical reaction under the action of the enzyme has high specificity, and each of the enzymes generally catalyzes only a constant reaction of a constant substrate. If such a characteristic of an enzyme, in particular an oxidoreductase can be ideally utilized for an oxidation-reduction reaction of an electrode, an electrode with low overvoltage and high selectivity can be created.

A constitution using two enzymes relating to associated reactions has been proposed as a technique for achieving an electron transport reaction with low overvoltage of the enzyme electrode. That is to say, an enzyme electrode, which simultaneously uses an enzyme to catalyze a reaction for producing a reaction product 1 from a reaction substrate 1 and an enzyme 2 to catalyze a reaction for producing a reaction product 2 from a reaction substrate 2, wherein a part of the reaction product 1 includes a part of the reaction substrate 2 has been proposed.

For example, such an electrode can include an example as described below.

In Ikura et al. (Japanese Patent Application Laid-Open No. 2003-279525), at least one part of a reaction layer including diaphorase, dehydrogenase and nicotinamide-adeninedinucleotidesynthetase is formed on a reaction electrode. Also, Ikura et al. discloses an enzyme electrode, wherein these enzymes are immobilized on the surface of the reaction electrode.

### SUMMARY OF THE INVENTION

As described above, an enzyme, which uses an enzyme to catalyze a reaction for producing a reaction product 1 from a reaction substrate 1 and an enzyme 2 to catalyze a reaction for producing a reaction product 2 from a reaction substrate 2 in the same reaction layer, is known. This enzyme is thermodynamically advantageous in an electron transfer reaction with low overvoltage from the reaction substrate 1 to the electrode. However, a relative distance and an orientation of the enzyme 1 and the enzyme 2 are random, and a diffusion process till one part of the reaction product 1 produced by the enzyme 1 is utilized as one part of the reaction substrate 2 of the enzyme 2 is a rate-determining step. Therefore, the entire electrode transfer reaction from the reaction substrate 1 to the electrode is not optimized in terms of reaction speed or reaction efficiency in the presence of a substrate with low density. That is to say, when such a conventional electrode is used as a sensor, a measurement value of current observed at the electrode together with an oxidation reaction of the reaction substrate 1 is small and sensitivity to the reaction substrate 1 is low, which causes a problem.

When the above-mentioned enzyme electrode using two enzymes is used as an electrode of a biofuel cell, output as a battery is small, and deterioration of the output is fast, which causes a problem.

Additionally, to improve sensitivity as a sensor, and to improve the output of the biofuel cell, it is normally considered that densities of the enzyme 1 and the enzyme 2 are made high and contained in the same reaction layer. In this case, a large amount of expensive enzyme has to be used, which is a factor for increasing costs of the enzyme electrode itself.

According to a first feature of the present invention, in an enzyme electrode having a conductive base plate, and an enzyme electrically connected with the conductive base plate, the enzyme is comprised of a fusion protein of a first enzyme to catalyze a chemical reaction for producing a first reaction product from a first reaction substrate and a second enzyme to catalyze a chemical reaction for producing a second reaction product from a second substrate, and at least one part of the first reaction product is identical to at least one part of the second reaction substrate.

It is preferable that the enzyme is configured to be immobilized to the conductive base plate together with an electron transfer mediator.

According to a second feature of the present invention, in a sensor, the enzyme electrode with the above-mentioned structure is used as a detection portion for detecting a base plate.

According to a third feature of the present invention, in a fuel cell of the present invention, the enzyme electrode with the above-mentioned structure is used as an anode.

According to a fourth feature of the present invention, in an electrochemical reaction device of the present invention, the enzyme electrode with the above-mentioned structure is used as a reaction pole.

In the enzyme electrode according to the present invention, a relative distance between the enzyme 1 and the enzyme 2 constituting the fusion protein is small, so that the electron transfer reaction from the reaction substrate 1 of the enzyme 1 to the electrode efficiently advances.

Therefore, the sensor using the enzyme electrode according to the present invention has a large current value together with oxidation of the substrate and high sensitivity. Also, in the fuel cell using the enzyme electrode according to the present invention, a high current value can be picked up. Furthermore, the electrochemical reaction device using the enzyme electrode according to the present invention shows high reaction efficiency.

Also, the enzyme electrode according to the present invention using an enzyme derived from a thermophile has superior storage stability compared to a conventional enzyme electrode.

The sensor, the fuel cell and the electrochemical reaction device using the enzyme electrode according to the present invention using the enzyme derived from the thermophile can be operated at high temperature. By the operation at high temperature, a diffusion rate-determining step is accelerated, so that larger sensitivity and output can be obtained.

Furthermore, an oxidoreductase used for the enzyme electrode according to the present invention does not require complicated operations such as a chromatography, and can be purified to high purity by a simple warming operation.

Further features of the present invention will become apparent from the following description of exemplary embodiments with reference to the attached drawings.

### BRIEF DESCRIPTION OF THE DRAWINGS

FIG. 1 is a schematic view of an enzyme electrode, which is comprised of a fusion protein of glucose dehydrogenase (GDH) and diaphorase (Dp) in one embodiment of the enzyme electrode according to the present invention.

FIG. 2 is a schematic view of an enzyme electrode, which is comprised of a fusion protein of alcohol dehydrogenase (ADH) and diaphorase (Dp) in one embodiment of the enzyme electrode according to the present invention;

FIG. 3 is a schematic view of an enzyme electrode, which is comprised of a fusion protein of lactic dehydrogenase (LDH) and diaphorase (Dp) in one embodiment of the enzyme electrode according to the present invention.

FIG. 4 is a schematic view of an enzyme electrode, which is comprised of a fusion protein of malic dehydrogenase (MDH) and diaphorase (Dp) in one embodiment of the enzyme electrode according to the present invention.

FIG. 5 is a schematic view of an enzyme electrode, which is comprised of a fusion protein of glutamic dehydrogenase (EDH) and diaphorase (Dp) in one embodiment of the enzyme electrode according to the present invention.

FIG. 6 is a schematic view of an enzyme electrode, which is comprised of a fusion protein of alcohol dehydrogenase (ADH) and aldehyde dehydrogenase (ALDH) in one embodiment of the enzyme electrode according to the present invention.

FIG. 7 is a schematic view of an enzyme electrode, which is comprised of a fusion protein of isomerase (ISO) and glucosededehydrogenase (GDH) in one embodiment of the enzyme electrode according to the present invention.

FIG. 8 is a schematic view of a sensor constituted using an enzyme electrode according to the present invention.

FIG. 9 is a schematic view of a fuel cell constituted using an enzyme electrode according to the present invention.

FIG. 10 is a schematic view of an enzyme electrode part, which is comprised of a fusion protein of glucose dehydrogenase and diaphorase in an embodiment of the present invention.

FIG. 11 shows a relationship between a substrate concentration and a current density measured using a glucose sensor in Embodiment 2.

FIG. 12 is a schematic view of an enzyme electrode part, which is comprised of glucose dehydrogenase and diaphorase in a comparison example of the present invention.

### DESCRIPTION OF THE EMBODIMENTS

An enzyme electrode according to the present invention comprises a conductive base plate, an enzyme immobilized to the conductive base plate, and an electron transfer mediator. Also, the enzyme only has to be immobilized to the conductive base plate and the electron transfer mediator only has to be used if needed, so that they are not essential to the present invention.

The present invention contains at least a fusion protein of two enzymes 1 and 2 having following relationships.

(A) The enzyme 1 catalyzes a reaction for producing a reaction product 1 from a reaction substrate 1.

(B) The enzyme 1 catalyzes a reaction for producing a reaction product 2 from a reaction substrate 2.

(C) The reaction product 1 becomes the reaction substrate 2 (The reaction product 1, which becomes the reaction substrate 2, may be one kind of a compound, or two or more kinds of compounds).

A preferable combination of enzymes to be fused can include combinations as follows.

(1) The combination of dehydrogenase (enzyme 1) and diaphorase (enzyme 2).

In this case, glucose dehydrogenase, alcohol dehydrogenase, lactic dehydrogenase, malic dehydrogenase or glutamic dehydrogenase is preferable as dehydrogenase.

(2) The combination of alcohol dehydrogenase (enzyme 1) and aldehyde dehydrogenase (enzyme 2).

In this case, it is preferable that diaphorase is made to coexist and immobilized on the conductive base plate.

(3) The combination of isomerase (enzyme 1) and glucosededehydrogenase (enzyme 2).

In this case, it is preferable that diaphorase is made to coexist and immobilized on the conductive base plate.

More preferable fusion protein can include those described as follows.

(A) A fusion protein having the following amino acid sequence (a), (b), (c) or (d), and having glucosededehydrogenase activity and diaphorase activity.

(a) An amino acid sequence expressed by SEQ ID NO:11.

(b) An amino acid sequence expressed by SEQ ID NO:11, wherein modifications such as addition, deletion and substitution of one or more amino acids are implemented independently or in combination of a plurality of modifications within a range not to deteriorate each of the above-mentioned activities.

(c) An amino acid sequence expressed by SEQ ID NO:23.

(d) An amino acid sequence expressed by SEQ ID NO:23, wherein modifications such as addition, deletion and substitution of one or more amino acids are implemented independently or in combination of a plurality of modifications.

(B) A fusion protein having the following amino acid sequence (e), (f), (g) or (h), and having alcohol dehydrogenase activity and diaphorase activity.

(e) An amino acid sequence expressed by SEQ ID NO:35.

(f) An amino acid sequence expressed by SEQ ID NO:35, wherein modifications such as addition, deletion and substitution of one or more amino acids are implemented independently or in combination of a plurality of modifications within a range not to deteriorate each of the above-mentioned activities.

(g) An amino acid sequence expressed by SEQ ID NO:44.

(h) An amino acid sequence expressed by SEQ ID NO:44, wherein modifications such as addition, deletion and substitution of one or more amino acids are implemented independently or in combination of a plurality of modifications within a range not to deteriorate each of the above-mentioned activities.

(C) A fusion protein having the following amino acid sequence (i), (j), (k) or (l), and having lactic dehydrogenase activity and diaphorase activity.

(i) An amino acid sequence expressed by SEQ ID NO:57.

(j) An amino acid sequence expressed by SEQ ID NO:57, wherein modifications such as addition, deletion and substitution of one or more amino acids are implemented independently or in combination of a plurality of modifications within a range not to deteriorate each of the above-mentioned activities.

(k) An amino acid sequence expressed by SEQ ID NO:69.

(l) An amino acid sequence expressed by SEQ ID NO:69, wherein modifications such as addition, deletion and substitution of one or more amino acids are implemented independently or in combination of a plurality of modifications within a range not to deteriorate each of the above-mentioned activities.

(D) A fusion protein having the following amino acid sequence (m), (n), (o) or (p), and having malic dehydrogenase activity and diaphorase activity.

(m) An amino acid sequence expressed by SEQ ID NO:79.

(n) An amino acid sequence expressed by SEQ ID NO:79, wherein modifications such as addition, deletion and substitution of one or more amino acids are implemented independently or in combination of a plurality of modifications within a range not to deteriorate each of the above-mentioned activities.

(o) An amino acid sequence expressed by SEQ ID NO: 88.

(p) An amino acid sequence expressed by SEQ ID NO:88, wherein modifications such as addition, deletion and substitution of one or more amino acids are implemented independently or in combination of a plurality of modifications within a range not to deteriorate each of the above-mentioned activities.

(E) A fusion protein having the following amino acid sequence (q), (r), (s) or (t), and having glutamic dehydrogenase activity and diaphorase activity.

(q) An amino acid sequence expressed by SEQ ID NO: 97.

(r) An amino acid sequence expressed by SEQ ID NO:97, wherein modifications such as addition, deletion and substitution of one or more amino acids are implemented independently or in combination of a plurality of modifications within a range not to deteriorate each of the above-mentioned activities.

(s) An amino acid sequence expressed by SEQ ID NO:106.

(t) An amino acid sequence expressed by SEQ ID NO:106, wherein modifications such as addition, deletion and substitution of one or more amino acids are implemented independently or in combination of a plurality of modifications within a range not to deteriorate each of the above-mentioned activities.

(F) A fusion protein having the following amino acid sequence (u), (v), (w), or (x), and having alcohol dehydrogenase activity and aldehyde dehydrogenase activity.

(u) An amino acid sequence expressed by SEQ ID NO:114.

(v) An amino acid sequence expressed by SEQ ID NO:114, wherein modifications such as addition, deletion and substitution of one or more amino acids are implemented independently or in combination of a plurality of modifications within a range not to deteriorate each of the above-mentioned activities.

(w) An amino acid sequence expressed by SEQ ID NO:121.

(x) An amino acid sequence expressed by SEQ ID NO:121, wherein modifications such as addition, deletion and substitution of one or more amino acids are implemented independently or in combination of a plurality of modifications within a range not to deteriorate each of the above-mentioned activities.

(G) A fusion protein having the following amino acid sequence (y), (z), (aa) or (ab), and having isomerase activity and glucosededehydrogenase activity.

(y) An amino acid sequence expressed by SEQ ID NO:128.

(z) An amino acid sequence expressed by SEQ ID NO:128, wherein modifications such as addition, deletion and substitution of one or more amino acids are implemented independently or in combination of a plurality of modifications within a range not to deteriorate each of the above-mentioned activities.

(aa) An amino acid sequence expressed by SEQ ID NO:135.

(ab) An amino acid sequence expressed by SEQ ID NO:135, wherein modifications such as addition, deletion and substitution of one or more amino acids are implemented independently or in combination of a plurality of modifications within a range not to deteriorate each of the above-mentioned activities.

The sensor, the fuel cell and the electrochemical reaction device can be constituted using the enzyme electrode with the above-mentioned structure.

The conductive base plate of the enzyme electrode according to the present invention is electrically connected with an external circuit when the enzyme electrode is used. Such a conductive base plate can be used, which has at least a conductive portion electrically connectable with the external circuit on an interface to an immobilized enzyme, has sufficient rigidity at the time of storage and measurement, and has sufficient electrochemical stability when the electrode is used.

For example, materials capable of constituting the conductive base plate can include metal, conductive polymer, metal oxide and carbon materials.

For example, the metal can include at least one kind of elements such as Au, Pt and Ag, and may include their alloys. Also, an appropriate base material may be plated with such metal so as to form a conductive base plate.

For example, the conductive polymer can include at least one of compounds such as polyacetylenes and polyarylenes.

For example, the metal oxide can include at least one kind of elements among In, Sn, Zn, Ti, Al, Si, Zr, Nb, Mg, Ba, Mo, W, V and Sr. When the portion made of the metal oxide does not have conductivity, conductivity can be given to that portion by other conductive materials. Also, even when the portion made of the metal oxide has conductivity, conductivity of that portion may be improved by other conductive materials. For example, the conductive material for giving conductivity to the metal oxide or for improving the conductivity of the metal oxide includes metal, conductive polymer, carbon materials and the like. For example, the carbon materials used for the conductive base plate include graphite, carbon black, carbon nanotube, carbon nanohorn, fullerene compound, and their derivatives. Also, when the portion made of the carbon materials does not have conductivity, conductivity can be given to that portion by other conductive materials. Even when the portion made of the carbon materials has conductivity, the conductivity of that portion can be improved by other conductive materials.

The conductive base plate may have voids at least at one part. The voids may be connected in a one-dimensional, two-dimensional or three-dimensional manner. For example, the voids connected in a one-dimensional manner can include columnar voids. For example, the voids connected in a two-dimensional manner can include net-like voids. For example, the voids connected in a three-dimensional manner can include voids formed after sponge-like fine particles are jointed, and voids with a structural material made using them as a template. It is preferable that such voids are large enough to introducer enzyme, and/or to sufficiently fluidize and diffuse matrices, and small enough to obtain a sufficient ratio of an effective surface area to a projection area. For example, the average diameter of the voids may include a range from 5 nanometers to 500 micrometers, and more preferably from 10 nanometers to 10 micrometers. Also, it is preferable that the thickness of the conductive base plate having the voids is small enough to constantly introduce the enzyme to a deep part of the conductive base plate, and/or to sufficiently fluidize and diffuse the matrices, and large enough to obtain a sufficient ratio of the effective surface ratio of the conductive base plate to the projection area. For example, the thickness of the conductive base plate having the voids may be from 100 nanometers to 1 centimeter and more preferably from 1 micrometer to 5 millimeters. The ratio of the effective surface of the effective surface area to the projection area of the conductive base plate having the voids needs to be large enough to obtain a sufficient ratio of the effective surface area to the projection area. For example, the ratio is 10 times or more, and more preferably 100 times or more.

It is preferable that porosity of the conductive base plate having the voids is set to satisfy at least one of following requirements (1) to (3) and a requirement (4).

(1) The porosity is large enough to obtain a sufficient ratio of the effective surface area to the projection area of a conductive member.

(2) The porosity is large enough to introduce a sufficient volume of enzymes and carriers.

(3) The porosity is large enough to sufficiently fluidize and diffuse the matrices.

(4) The porosity is small enough to obtain a sufficient mechanical strength.

For example, the porosity is 20% or more and 99% or less, and more preferably 30% or more and 98% or less.

A metallic conductive base plate having a large number of voids includes foamed metal, electrocrystallized metal, electrolytic metal, sintered metal, fibrous metal, or materials applied to one kind or a plurality of kinds of them.

A method used for manufacturing porous resin such as following methods can be exemplified as an example a method for manufacturing conductive polymer having a large number of voids.

(1) A method for locating a substance as a mold constituting a portion to be a void in conductive polymer and forming it into a predetermined shape, and then removing the substance as the mold.

(2) A method for containing a substance as a mold of a portion to be a void in a precursor of conductive polymer and polymerizing the precursor into polymer, and then removing the substance as the mold.

(3) A method for forming a layer made of particles to be a mold constituting a portion to be a void, filling the void between the particles with a precursor of polymer to form a layer, and removing the particle from the layer.

(4) A method for forming a layer made of particles to be a mold constituting a portion to be a void, filling the void between the particles with a precursor of polymer to form a layer, polymerizing the precursor into a polymer layer, and the removing the particles.

For example, a method for manufacturing metal oxide having a large number of voids can include methods such as electrocrystallization, spattering, sintering, chemical vapor deposition (CVD), electrolysis, and their combinations.

For example, a method for manufacturing a carbon material having a large number of voids includes the following method. That is to say, it can include a method for molding fibers or particles made of graphite, carbon black, carbon nanotube, carbon nanohorn, fullerene compound, and their derivatives into a predetermined shape, and sintering them.

In the enzyme electrode according to the present invention, the enzyme is immobilized adjacent to the conductive base plate. That is to say, an immobilized enzyme layer is laminated directly on a surface having conductivity of the conductive base plate. In this manner, the immobilized enzyme layer is formed directly on the conductive base plate, so that fusion protein and an electron transfer mediator can be captured physically near the conductive base plate. As a result, a quick electron transfer reaction between the enzyme and the conductive base plate via the electron transfer mediator can be promoted, and furthermore, the fusion protein and the electron transfer mediator can be prevented from being scattered from the vicinity of the conductive base plate. Furthermore, such a constitution makes it possible to repeatedly use the enzyme electrode, and improve durability of the enzyme electrode.

Enzymes can be immobilized by a method known to those skilled in the art used for capturing the fusion protein physically near the conductive base plate. Concrete methods for creating the immobilized enzyme layer can include, for example, following methods (1) to (6).

(1) Covalent Binding Method

Functional groups are introduced directly on the surface of the conductive base plate, and the functional groups are covalent bound with the enzymes, so that the enzymes are immobilized. Or, functional groups are introduced to carriers located in contact with the conductive base plate, and the functional groups are covalently bound with the enzymes, so that the enzymes are immobilized.

Such functional groups which can be used for covalent binding can include, for example, a hydroxyl group, a carboxyl group, an amino group and the like.

Also, by using a fact that a thiol group of alkylthiol acts on metal such as gold, and is easily bound so as to easily create a monomolecular film (a self-organized monomolecular film), the enzymes are bound with the metal by covalent binding via the functional groups introduced to an alkyl group of alkylthiol in advance, so as to immobilize the enzymes.

The functional groups introduced to the alkyl group of alkylthiol can be covalently bound with the enzymes, for example, using a bifunctional reagent. Representative bifunctional reagents can include glutaraldehyde, periodic acid and the like.

Also, the carriers located in contact with the conductive base plate can include agarose, agarose resolvent, κ-carrageenan, agar, alginic acid, polyacrylamide, polyisopropylacrylamide, polyvinyl alcohol, their copolymers and the like.

(2) Crosslinking Method

The enzymes are crosslinked and bound with each other and immobilized using a crosslinking agent such as glutaraldehyde. Also, a substrate substance such as gelatin and albumin is added to the enzymes to form the crosslinking between the enzymes and the substrate substance, so that the enzymes are immobilized together with the substrate substance. At the time of immobilization, synthetic polymers such as polyallylamine and polylysine are made to coexist, so that characteristics of the enzyme immobilized layer, i.e., film strength, a substrate transmission characteristic and the like may be controlled.

(3) Inclusion Method

The enzymes are included in agarose, agarose resolvent, κ-carrageenan, agar, their copolymers and the like, so that the enzymes are immobilized.

(4) Adsorption Method (No. 1)

The enzymes are immobilized by a physical adsorption method utilizing a hydrophobic interaction of the conductive base plate and the enzymes. When physical adsorption of the enzymes to the conductive base plate is impossible or insufficient, the enzymes are immobilized via a carrier for physically adsorbing the enzymes. This carrier can include carriers made of polyallylamine, polylysine, polyvinyl pyridine, dextran denatured with amino groups (for example, DEAE-dextran), chitosan, polyglutamates, polystyrene sulphonic acid, polyanion such as dextran sulfate, and polycation. The enzymes are immobilized to the carrier by an ion-bonding method utilizing an electrostatic interaction of the carrier and the enzymes, and the enzyme-immobilized carrier is located in contact with the conductive base plate.

(5) Diaphragm Process

An enzyme solution is coated and immobilized on the conductive base plate using a transmission-limiting film such as a polyimide film, an accetylcellulose film, a polysulfone film, a perfluorosulfone acid polymer film (e.g., Commodity Name “Nafion” manufactured by DuPont.) as a diaphragm.

(6) Adsorption Method (No. 2)

The enzymes are immobilized utilizing various kinds of affinity tags used for facilitating purification of genetically-modified proteins. For example, the enzymes are immobilized utilizing epitope tags such as hemagglutinin (HA), FLAG and Myc, GST, maltose-combined protein, biotinylated peptide and oligohistidine tags.

An immobilization volume of the enzymes in the enzyme electrode according to the present invention is not specially limited, and can be changed in a wide range.

The fusion protein contained in the enzyme electrode according to the present invention is a multifunctional enzyme having the enzyme activity of the enzyme 1 and the enzyme activity of the enzyme 2. In this fusion protein, at least one chemical substance of the reaction product 1 produced by the enzyme activity of the enzyme 1 is quickly utilized as a reaction substrate of the enzyme 2 existing physically near the enzyme 1. As a result, the enzyme activity to produce the reaction product 2 from the reaction substrate 1 of the fusion protein is higher when the enzyme 1 and the enzyme 2 exist separately.

The concrete combination of the enzyme 1 and the enzyme 2 is not especially limited as long as at least one chemical substance of the reaction product 1 of the enzyme 1 is identical to at least one chemical substance of the reaction substrate 2 of the enzyme 2 and has activity as an enzyme for an enzyme electrode.

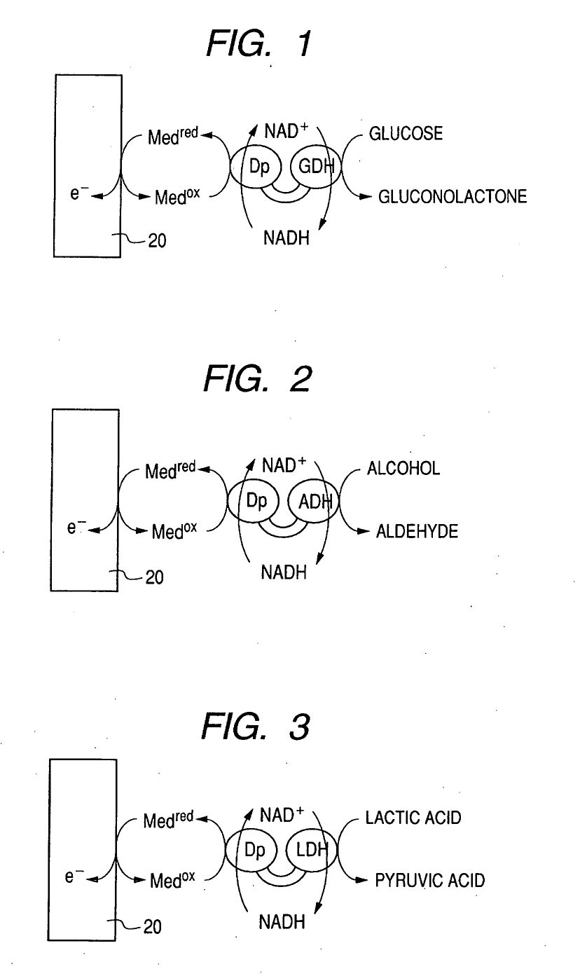

Among others, when the enzyme 1 is dehydrogenase and the enzyme 2 is diaphorase, influences of oxygen to an electrochemical response observed at the enzyme electrode together with the oxidation reaction of the substrate can be reduced, which is suitable. Additionally, most of dehydrogenase utilizes nicotinamide adenine dinucleotide or nicotinamide adenine dinucleotide phosphate as a receptor of an electron and a hydrogen atom. Therefore, by selecting a kind of dehydrogenase fused with diaphorase, a sensor with high versatility to a detection object can be manufactured. For example, such dehydrogenase can include glucose dehydrogenase, alcohol dehydrogenase, glutamic dehydrogenase, cholesterol dehydrogenase, aldehyde dehydrogenase, fructosedehydrogenase, sorbitoldehydrogenase, lactic dehydrogenase, malic dehydrogenase, glyceroldehydrogenase, 17B-hydroxysteroiddehydrogenase, estradiol 17B-dehydrogenase, amino-acid dehydrogenase, glyceraldehyde 3-phosphate dehydrogenase, 3-hydroxysteroiddehydrogenase and the like. For example, when the enzyme electrode which is made of these fusion protein is manufactured using glucose dehydrogenase (GDH) as the enzyme 1 and diaphorase (Dp) as the enzyme 2, a condition shown in FIG. 1 can be obtained.

That is to say, the enzyme immobilized layer having the fusion protein of glucose dehydrogenase GDH and diaphorase Dp and an electron transfer mediator Med is located in contact with the conductive base plate 20. When glucose is made to act on the enzyme electrode, glucose is oxidized by a catalytic action of glucose dehydrogenase in the coexistence of nicotinamide adenine dinucleotide (NAD⁺) and the like. As a result, gluconolactone and reduced nicotinamide adenine dinucleotide (NADH) are produced.

Then, the reduced nicotinamide adenine dinucleotide is immediately oxidized by a catalytic action of diaphorase existing physically near glucose dehydrogenase in the presence of an oxidized electron transfer mediator (Med^{OX}).

As a result, nicotinamide adenine dinucleotide and a reduced electron transfer mediator (Med^{red}) are produced. The resultant reduced electron transfer mediator (Med^{red}) can transfer electrons to the conductive base plate.

In FIG. 1, first reaction matrices in a first feature of the present invention are glucose and NAD⁺, and first reaction products are gluconolactone and NADH. NADH is also a second reaction substrate, and NAD⁺ is also a second reaction product.

That is to say, NADH which is a part of the first reaction products is identical to at least one part of second reaction matrices.

In the enzyme electrode according to the present invention, electrons can be transferred to the electrode from glucose more efficiently than a conventionally-known case where glucose dehydrogenase and diaphorase are independently immobilized on the electrode. Accordingly, the enzyme electrode shown in FIG. 1 can be utilized as a glucose sensor with high detection sensitivity, a glucose fuel cell with large output, and a glucose electrochemical reaction device with high reaction efficiency.

Especially by using the fusion protein of glucose dehydrogenase and diaphorase derived from thermophile, the enzyme electrode with excellent thermal resistance, durability and response in a high temperature condition can be manufactured.

When the enzyme electrode is manufactured using alcohol dehydrogenase as the enzyme 1 and diaphorase as the enzyme 2, a condition shown in FIG. 2 can be obtained. That is to say, the enzyme immobilized layer having the fusion protein of alcohol dehydrogenase ADH and diaphorase Dp and an electron transfer mediator Med is located in contact with the conductive base plate. When alcohol is made to act on the enzyme electrode, alcohol is oxidized by a catalytic action of alcohol dehydrogenase in the coexistence of nicotinamide adenine dinucleotide and the like. As a result, aldehyde and reduced nicotinamide adenine dinucleotide are produced. Then, the reduced nicotinamide adenine dinucleotide is immediately oxidized by a catalytic action of diaphorase existing physically near alcohol dehydrogenase in the presence of an oxidized electron transfer mediator. As a result, nicotinamide adenine dinucleotide and a reduced electron transfer mediator are produced. The resultant reduced electron transfer mediator can transfer electrons to the conductive base plate.

When the enzyme electrode is manufactured using lactic dehydrogenase as the enzyme 1 and diaphorase as the enzyme 2, a condition shown in FIG. 3 can be obtained. That is to say, the enzyme immobilized layer having the fusion protein of lactic dehydrogenase LDH and diaphorase Dp and an electron transfer mediator Med is located in contact with the conductive base plate. When lactic acid is made to act on the enzyme electrode, lactic acid is oxidized by a catalytic action of lactic dehydrogenase in the coexistence of nicotinamide adenine dinucleotide and the like. As a result, pyruvic acid and reduced nicotinamide adenine dinucleotide are produced. Then, the reduced nicotinamide adenine dinucleotide is immediately oxidized by a catalytic action of diaphorase existing physically near lactic dehydrogenase in the presence of an oxidized electron transfer mediator. As a result, nicotinamide adenine dinucleotide and a reduced electron transfer mediator are produced. The resultant reduced electron transfer mediator can transfer electrons to the conductive base plate.

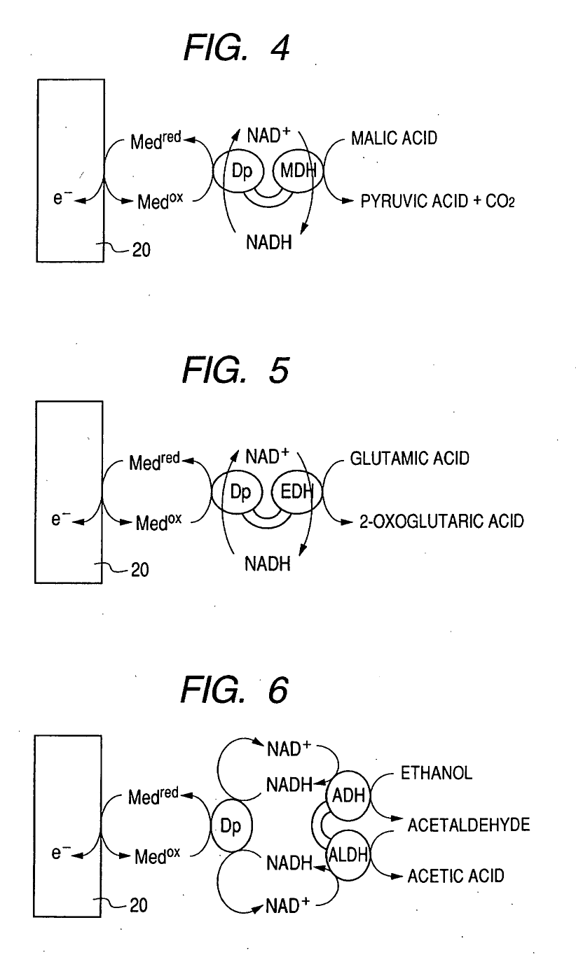

When the enzyme electrode is manufactured using malic dehydrogenase as the enzyme 1 and diaphorase as the enzyme 2, a condition shown in FIG. 4 can be obtained. That is to say, the enzyme immobilized layer having the fusion protein of malic dehydrogenase MDH and diaphorase Dp and an electron transfer mediator Med is located in contact with the conductive base plate. When malic acid is made to act on the enzyme electrode, malic acid is oxidized by a catalytic action of malic dehydrogenase in the coexistence of nicotinamide adenine dinucleotide and the like. As a result, pyruvic acid and reduced nicotinamide adenine dinucleotide are produced. Then, the reduced nicotinamide adenine dinucleotide is immediately oxidized by a catalytic action of diaphorase existing physically near malic dehydrogenase in the presence of an oxidized electron transfer mediator. As a result, nicotinamide adenine dinucleotide and a reduced electron transfer mediator are produced. The resultant reduced electron transfer mediator can transfer electrons to the conductive base plate.

When the enzyme electrode is manufactured using glutamic dehydrogenase as the enzyme 1 and diaphorase as the enzyme 2, a condition shown in FIG. 5 can be obtained. That is to say, the enzyme immobilized layer having the fusion protein of glutamic dehydrogenase EDH and diaphorase Dp and an electron transfer mediator Med is located in contact with the conductive base plate. When glutamic acid is made to act on the enzyme electrode, glutamic acid is oxidized by a catalytic action of glutamic dehydrogenase in the coexistence of nicotinamide adenine dinucleotide and the like. As a result, 2-oxoglutaric acid and reduced nicotinamide adenine dinucleotide are produced. Then, the reduced nicotinamide adenine dinucleotide is immediately oxidized by a catalytic action of diaphorase existing physically near glutamic dehydrogenase in the presence of an oxidized electron transfer mediator. As a result, nicotinamide adenine dinucleotide and a reduced electron transfer mediator are produced. The resultant reduced electron transfer mediator can transfer electrons to the conductive base plate.

In the enzyme electrode according to the present invention, electrons can be transferred to the electrode from glutamic acid more efficiently than a conventionally-known case where glutamic dehydrogenase and diaphorase are independently immobilized on the electrode. Accordingly, the enzyme electrode shown in FIG. 5 can be utilized as a glutamic acid sensor with high detection sensitivity, a glutamic acid fuel cell with large output, and a glutamic acid electrochemical reaction device with high reaction efficiency.

Especially by using the fusion protein of glutamic dehydrogenase and diaphorase derived from thermophile, the enzyme electrode with excellent thermal resistance, durability and response in a high temperature condition can be manufactured.

When the enzyme electrode is manufactured using alcohol dehydrogenase as the enzyme 1 and aldehyde dehydrogenase as the enzyme 2 and in the coexistence of diaphorase, a condition shown in FIG. 6 can be obtained. That is to say, the enzyme immobilized layer having the fusion protein of alcohol dehydrogenase ADH and aldehyde dehydrogenase ALDH, diaphorase Dp and an electron transfer mediator Med is located in contact with the conductive base plate. When alcohol is made to act on the enzyme electrode, alcohol is oxidized by a catalytic action of alcohol dehydrogenase in the coexistence of nicotinamide adenine dinucleotide and the like. As a result, aldehyde and reduced nicotinamide adenine dinucleotide are produced. Then, the resultant aldehyde is immediately oxidized by a catalytic action of aldehyde dehydrogenase existing physically near alcohol dehydrogenase in the coexistence of nicotinamide adenine dinucleotide and the like. As a result, carboxylic acid and reduced nicotinamide adenine dinucleotide are produced. The resultant reduced nicotinamide adenine dinucleotide is oxidized by a catalytic action of diaphorase in the presence of an oxidized electron transfer mediator. As a result, nicotinamide adenine dinucleotide and a reduced electron transfer mediator are produced. The resultant reduced electron transfer mediator can transfer electrons to the conductive base plate. Additionally, this enzyme electrode can prevent aldehyde species from being accumulated near the enzyme electrode, and reduce an inactivation reaction of enzyme protein by aldehyde species, so that deterioration of the activities of the enzyme electrode can be restrained.

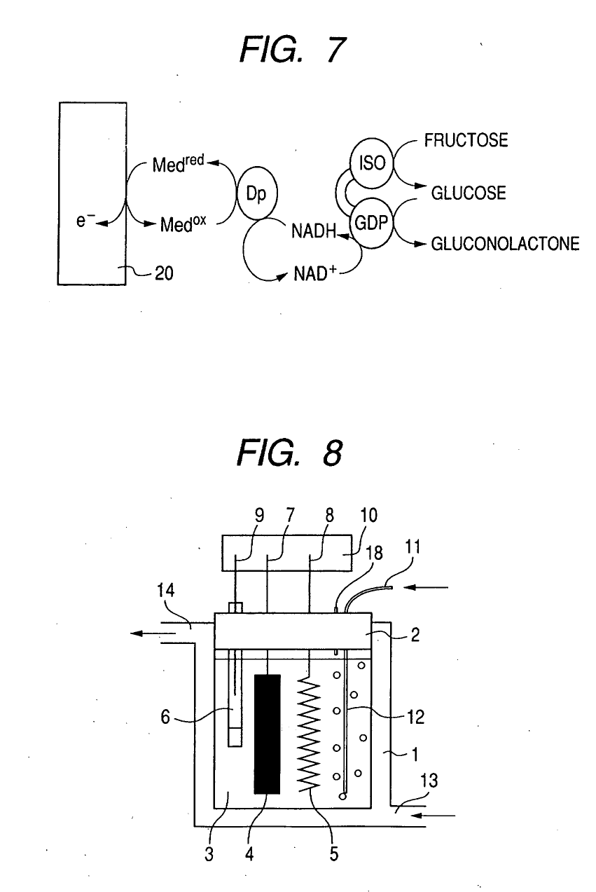

When the enzyme electrode is manufactured using isomerase as the enzyme 1 and glucosededehydrogenase as the enzyme 2 and in the coexistence of diaphorase, a condition shown in FIG. 7 can be obtained. That is to say, the enzyme immobilized layer having the fusion protein of isomerase ISO and glucosededehydrogenase GDH, diaphorase Dp and an electron transfer mediator Med is located in contact with the conductive base plate. When fructose is made to act on the enzyme electrode, fructose is isomerized by a catalytic action of isomerase so as to produce glucose. The produced glucose is immediately oxidized by a catalytic action of glucosededehydrogenase existing physically near isomerase in the coexistence of nicotinamide adenine dinucleotide and the like. As a result, gluconolactone and reduced nicotinamide adenine dinucleotide are produced. The resultant reduced nicotinamide adenine dinucleotide is oxidized by a catalytic action of diaphorase in the presence of an oxidized electron transfer mediator. As a result, nicotinamide adenine dinucleotide and a reduced electron transfer mediator are produced. The resultant reduced electron transfer mediator can transfer electrons to the conductive base plate.

The fusion protein used for the enzyme electrode according to the present invention can be manufactured using a genetic engineering technique. First, a first DNA sequence having genes for coding the amino acid sequence of the enzyme 1 and a second DNA sequence having genes for coding the amino acid sequence of the enzyme 2 are prepared. Then, the first DNA sequence and the second DNA sequence are bounded with each other under a condition that the fusion protein with these enzymes fused can be expressed. For example, the second DNA sequence is connected with the upstream of the 5′ side or the downstream of the 3′ side of the first DNA, so as to obtain the DNA sequence as recombination genes for expressing the fusion protein. The recombination genes are used for an appropriate host-vector system and expressed, so as to obtain the recombination genes coded with them. The expressed fusion protein is isolated and purified, so that it can be used for the enzyme electrode.

The genetic DNA sequences for coding the amino acid sequences of the enzyme 1 and the enzyme 2 are not especially limited to specific derivation, as long as their functions are identified.

As glucose dehydrogenase (EC1.1.1.47) for the fusion protein, a function for catalyzing the following chemical reaction is identified, and any known basic sequence of the gene or any known amino acid sequence of the enzyme can be used. That is to say, its derivation is not especially limited.
beta-D-glucose+NAD(P)⁺=D-glucono-1,5-lactone+NAD(P)H+H⁺ Reaction Formula 1:

Such glucose dehydrogenase genes can include Bacillus spp., for example Bacillus subtilis 168 [Nature 390:249-56 (1997)], Gloeobacter spp., for example Gloeobacter violaceus PCC7421 [DNA Res 10:137-45 (2003)], Thermoplasma spp., for example Thermoplasma acidophilum DSM 1728 [Nature 407:508-13 (2000)], Thermoplasma volcanium GSS1 [Proc Natl Acad Sci U.S.A. 97:14257-62 (2000)], Picrophilus spp., for example Picrophilus torridus DSM 9790 [Proc Natl Acad Sci U.S.A. 101:9091-6 (2004)], Pyrococcus subsp. Pyrococcus furiosus DSM 3638 [Genetics 152:1299-305 (1999)], and Sulfolobus spp., for example Sulfolobus solfataricus [Proc Natl Acad Sci U.S.A. 98:7835-40 (2001)], Sulfolobus tokodaii strain 7 [DNA Res 8:123-40 (2001)], any of which can be used as a component of the fusion protein according to the present invention. In particular, enzymes derived from Pyrococcus furiosus, Sulfolobus solfataricus and the like has thermal resistance, and can be suitably used for the present invention.

As alcohol dehydrogenase (EC1.1.1.1) for the fusion protein, a function for catalyzing the following chemical reaction is identified, and any known basic sequence of the gene or any known amino acid sequence of the enzyme can be used. That is to say, its derivation is not especially limited.
alcohol+NAD+=aldehyde or ketone+NADH+H+ Reaction Formula 2:

Such alcohol dehydrogenase genes can include Saccharomyces spp., for example Saccharomyces cerevisiae S288C [Science 274:546-67 (1996)), Pseudomonas spp., for example Pseudomonas aeruginosa PA01 [Nature 406:959-64 (2000)], Pseudomonas putida KT2440 [Environ Microbiol 4:799-808 (2002)], Acinetobacter spp., for example Acinetobacter sp. ADP1 [Nucleic Acids Res 32:5766-79 (2004)], Bacillus spp., for example Bacillus subtilis 168 [Nature 390:249-56 (1997)], Lactococcus spp., for example Lactococcus lactis subsp. lactis IL1403 [Genome Res 11:731-53 (2001)], Lactobacillus spp., for example Lactobacillus plantarum WCFS1 [Proc Natl Acad Sci U.S.A. 100:1990-5 (2003)], Thermus spp., for example Thermus thermophilus HB27 [Nat Biotechnol 22:547-53 (2004)], Aquifex spp., for example Aquifex aeolicus VF5 [Nature 392:353-8 (1998)], Thermotoga spp., for example Thermotoga maritima MSB8 [Nature 399:323-9 (1999)], Methanococcus spp., for example Methanococcus maripaludis S2 [J Bacteriol 186:6956-69 (2004)], Methanosarcina spp., for example Methanosarcina acetivorans C2A[Genome Res 12:532-42 (2002)], Methanosarcina mazei Goe1 [J Mol Microbiol Biotechnol 4:453-61 (2002)], Thermoplasma spp., for example Thermoplasma acidophilum DSM1728 [Nature 407:508-13 (2000)), Thermoplasma volcanium GSS1 [Proc Natl Acad Sci U.S.A. 97:14257-62 (2000)], Pyrococcus spp., for example Pyrococcus horikoshii OT3 [DNA Res 5:55-76 (1998)], Pyrococcus abyssi GE5, Pyrococcus furiosus DSM 3638 [Genetics 152:1299-305 (1999), Mol Microbiol 38:684-93 (2000), Methods Enzymol 330:134-57 (2001)], Aeropyrum spp., for example Aeropyrum pernix K1 [DNA Res 6:83-101, 145-52 (1999)], Sulfolobus spp., for example Sulfolobus solfataricus [Proc Natl Acad Sci U.S.A. 98:7835-40 (2001)], Sulfolobus tokodaii strain 7 [DNA Res 8:123-40 (2001)], and Pyrobaculum spp., for example Pyrobaculum aerophilum IM2 [Proc Natl Acad Sci U.S.A. 99:984-9 (2002)], any of which can be used for a component of the fusion protein according to the present invention. In particular, enzymes derived from Corynebacterium efficiens, Thermus thermophilus Aquifex aeolicus, Thermotoga maritima, Archaeoglobus fulgidus, Pyrococcus horikoshii, Pyrococcus abyssi, Pyrococcus furiosus, Aeropyrum pernix, Pyrobaculum aerophilum and the like has thermal resistance, and can be suitably used for the present invention.

As aldehyde dehydrogenase for the fusion protein, a function for catalyzing following chemical reactions is identified, and any known basic sequence of the gene or any known amino acid sequence of the enzyme can be used. That is to say, its derivation is not especially limited.

(1) Enzyme requiring NAD as an electron receptor (EC 1.2.1.3)
aldehyde+NAD⁺+H₂O=acid+NADH+H⁺ Reaction Formula 3:

(2) Enzyme requiring NAD or NADP as an electron receptor (EC 1.2.1.5)
aldehyde+NAD(P)⁺+H₂O=acid+NAD(P)H+H⁺ Reaction Formula 4:

In particular, as aldehyde dehydrogenase for oxidizing acetaldehyde, a function for catalyzing the following chemical reaction is identified, and any known basic sequence of the gene or any known amino acid sequence of the enzyme can be used. That is to say, its derivation is not especially limited.
acetaldehyde+CoA+NAD⁺=acetyl-CoA+NADH+H⁺ Reaction Formula 5:

In the case of aldehyde dehydrogenase (EC 1.2.1.3) requiring NAD as an electron receptor, such aldehyde dehydrogenase genes can include Acinetobacter spp., for example Acinetobacter sp. ADP1 [Nucleic Acids Res, 32:5766-79 (2004)], Bacillus spp., for example Bacillus subtilis 168 [Nature 390:249-56 (1997)], Thermus spp., for example Thermus thermophilus HB27 [Nat Biotechnol 22:547-53 (2004)], Pyrococcus spp., for example Pyrococcus furiosus DSM 3638 [Genetics 152:1299-305 (1999), Mol Microbiol 38:684-93 (2000), Methods Enzymol 330:134-57 (2001)], Aquifex spp., for example Aquifex aeolicus VF5 [Nature 392:353-8 (1998)], and the like, any of which can be used as a component of the fusion protein according to the present invention.

Also, in the case of aldehyde dehydrogenase (EC 1.2.1.5) requiring NAD or NADP as the electron receptor, such aldehyde dehydrogenase genes can include Caenorhabditis spp., for example Caenorhabditis elegans [Science 282:2012-8 (1998)], Bacillus spp., for example Bacillus thuringiensis 97-27 (serovar konkukian), and the like, any of which can be used for a component of the fusion protein according to the present invention.

Also, in the case of aceto aldehyde dehydrogenase (EC 1.2.1.10), the genes can include Bacillus spp., for example Bacillus cereus ATCC 14579 [Nature 423:87-91 (2003)], Bifidobacterium spp., for example Bifidobacterium longum NCC2705 [Proc Natl Acad Sci U.S.A. 99:14422-7 (2002)], and the like, any of which can be used for a component of the fusion protein according to the present invention.

In particular, enzymes derived from Thermus thermophilus, Pyrococcus furiosus, Aquifex aeolicus and the like has thermal resistance, and can be suitably used for the present invention.

As lactic dehydrogenase for the fusion protein (EC 1.1.1.27), a function for catalyzing the following chemical reaction is identified, and any known basic sequence of the gene or any known amino acid sequence of the enzyme can be used. That is to say, its derivation is not especially limited.
(S)-lactate+NAD⁺=pyruvate+NADH+H⁺ Reaction Formula 6:

Such lactic dehydrogenase genes can include Bacillus spp., for example Bacillus subtilis 168 [Nature 390:249-56 (1997)], Lactococcus spp., for example Lactococcus lactis subsp. lactis IL1403 [Genome Res 11:731-53 (2001)], Lactobacillus spp., for example Lactobacillus plantarum WCFS1 [Proc Natl Acad Sci U.S.A. 100:1990-5 (2003)], Lactobacillus johnsonii NCC 533 [Proc Natl Acad Sci U.S.A.: (2004)], Deinococcus spp., for example Deinococcus radiodurans R1 [Science 286:1571-7 (1999)], Thermus spp., for example Thermus thermophilus HB27 [Nat Biotechnol 22:547-53 (2004)], Thermotoga spp., for example Thermotoga maritima MSB8 [Nature 399:323-9 (1999)], and the like, any of which can be used for a component of the fusion protein according to the present invention. In particular, enzymes derived from Thermus thermophilus, Thermotoga maritima and the like has thermal resistance, and can be suitably used for the present invention.

Diaphorase activity is recognized as diaphorase for the fusion protein, and any known basic sequence of the genetic DNA or any known amino acid sequence of the enzyme can be used. That is to say, its derivation is not especially limited. The diaphorase activity is a catalytic reaction for oxidizing NADH or NADPH in the present of an artificial electron receptor such as methylene blue and 2,6-dichlorophenol-indophenol. The enzymes having such diaphorase activity are classified as follows depending on whether they have specificity in any one or both of NADH and NADPH.

(1) EC 1.6.99.1; NADPH: (acceptor) oxidoreductase

(2) EC 1.6.99.2; NAD(P)H: (quinone-acceptor) oxidoreductase

(3) EC 1.6.99.3; NADH: (acceptor) oxidoreductase

(4) EC 1.6.99.5; NADH: (quinone-acceptor) oxidoreductase

(5) EC 1.8.1.4; protein-N-6-(dihydrolipoyl) lysine:NAD+ oxidoreductase

(6) EC 1.14.13.39; L-arginine, NADPH:oxygen oxidoreductase (nitric-oxide-forming)

In the enzyme electrode according to the present invention, it is desirable to select and use diaphorase having a substrate specificity identical to dehydrogenase depending on which substrate specificity dehydrogenase fused or coexisted with diaphorase has in NADPH or NADH.

When the above-mentioned diaphorase (1) is used, function for catalyzing the following chemical reaction is identified, and any known basic sequence of the gene or any known amino acid sequence of the enzyme can be used. That is to say, its derivation is not especially limited.
NADPH+H⁺+acceptor=NADP⁺+reduced acceptor Reaction Formula 7:

Such diaphorase genes can include Saccharomyces spp., for example Saccharomyces cerevisiae S288C [Science 274:546-67 (1996), Proc Natl Acad Sci U.S.A. 92:3809-13 (1995), EMBO J. 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J. 15:2031-49 (1996), Nature 369:371-8 (1994), Nature 387:87-90 (1997), Nature 387:90-3 (1997), Nature 387:93-8 (1997), Nature 387:98-102 (1997), Nature 387:103-5 (1997)], Candida spp., for example Candida albicans SC5314 [Proc Natl Acad Sci U.S.A. 101:7329-34 (2004)] and the like.

When the above-mentioned diaphorase (2) is used, function for catalyzing the following chemical reaction is identified, and any known basic sequence of the gene or any known amino acid sequence of the enzyme can be used. That is to say, its derivation is not especially limited.
NAD(P)H+H⁺+acceptor=NAD(P)⁺+reduced acceptor Reaction Formula 8:

Such diaphorase genes can include Pseudomonas spp., for example Pseudomonas aeruginosa PA01 [Nature 406:959-64 (2000)], Pseudomonas putida KT2440 [Environ Microbiol 4:799-808 (2002)], Bacillus spp., for example Bacillus cereus ATCC 14579 [Nature 423:87-91 (2003)], and the like.

When the above-mentioned diaphorase (3) is used, function for catalyzing the following chemical reaction is identified, and any known basic sequence of the gene or any known amino acid sequence of the enzyme can be used. That is to say, its derivation is not especially limited.
NADH+H⁺+acceptor=NAD⁺+reduced acceptor Reaction Formula 9:

Such diaphorase genes can include Saccharomyces spp., for example Saccharomyces cerevisiae S288C [Science 274:546-67 (1996), Proc Natl Acad Sci U.S.A. 92:3809-13 (1995), EMBO J. 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J. 15:2031-49 (1996), Nature 369:371-8 (1994), Nature 387:87-90 (1997), Nature 387:90-3 (1997), Nature 387:93-8 (1997), Nature 387:98-102 (1997), Nature 387:103-5 (1997)], Pseudomonas spp., for example Pseudomonas aeruginosa PA01 [Nature 406:959-64 (2000)], Pseudomonas putida KT2440 [Environ Microbiol 4:799-808 (2002)], Acinetobacter spp., for example Acinetobacter sp. ADP1 [Nucleic Acids Res 32:5766-79 (2004)], Bacillus spp., for example Bacillus subtilis 168 [Nature 390:249-56 (1997)], Lactobacillus spp., for example Lactobacillus plantarum WCFS1 [Proc Natl Acad Sci U.S.A. 100:1990-5 (2003)], Deinococcus spp., for example Deinococcus radiodurans R1 [Science 286:1571-7 (1999)], Thermus spp., for example Thermus thermophilus HB27 [Nat Biotechnol 22:547-53 (2004)], Aquifex spp., for example Aquifex aeolicus VF5 [Nature 392:353-8 (1998)], Pyrococcus spp., for example Pyrococcus abyssi GE5, Pyrococcus furiosus DSM 3638 [Genetics 152:1299-305 (1999), Mol Microbiol 38:684-93 (2000), Methods Enzymol 330:134-57 (2001)], and the like.

When the above-mentioned diaphorase (4) is used, function for catalyzing the following chemical reaction is identified, and any known basic sequence of the gene or any known amino acid sequence of the enzyme can be used. That is to say, its derivation is not especially limited.
NADH+H⁺+acceptor=NAD++reduced acceptor Reaction Formula 10:

Such diaphorase genes can include Burkholderia spp., for example Burkholderia mallei ATCC 23344 [Proc Natl Acad Sci U.S.A. 101:14246-51 (2004)), Haloarcula spp., for example Haloarcula marismortui ATCC 43049 [Genome Res 14:2221-34 (2004)], and the like.

When the above-mentioned diaphorase (5) is used, function for catalyzing the following chemical reaction is identified, and any known basic sequence of the gene or any known amino acid sequence of the enzyme can be used. That is to say, its derivation is not especially limited.
protein N6-(dihydrolipoyl)lysine+NAD⁺=protein N6-(lipoyl)lysine+NADH+H⁺ Reaction Formula 11:

Such diaphorase genes can include Saccharomyces spp., for example Saccharomyces cerevisiae S288C [Science 274:546-67 (1996), Proc Natl Acad Sci U.S.A. 92:3809-13 (1995), EMBO J. 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J. 15:2031-49 (1996), Nature 369: 371-8 (1994), Nature 387:87-90 (1997), Nature 387:90-3 (1997), Nature 387:93-8 (1997), Nature 387:98-102 (1997), Nature 387:103-5 (1997)], Lactobacillus spp., for example Lactobacillus plantarum WCFS1 [Proc Natl Acad Sci U.S.A. 100:1990-5 (2003)], Deinococcus spp., for example Deinococcus radiodurans R1 [Science 286:1571-7 (1999)], Thermus spp., for example Thermus thermophilus HB27 [Nat Biotechnol 22:547-53 (2004)], Aquifex spp., for example Aquifex aeolicus VF5 [Nature 392:353-8 (1998)], Thermotoga spp., for example Thermotoga maritima MSB8 [Nature 399:323-9 (1999)], Sulfolobus spp., for example Sulfolobus solfataricus [Proc Natl Acad Sci U.S.A. 98:7835-40 (2001)], Sulfolobus tokodaii strain 7 [DNA Res 8:123-40 (2001)], Pyrobaculum spp., for example Pyrobaculum aerophilum IM2 [Proc Natl Acad Sci U.S.A. 99:984-9 (2002)], and the like.

When the above-mentioned diaphorase (6) is used, a function for catalyzing the following chemical reaction is identified, and any known basic sequence of the gene or any known amino acid sequence of the enzyme can be used. That is to say, its derivation is not especially limited.
L-arginine+nNADPH+nH⁺+mO₂=citrulline+nitric oxide+nNADP+ Reaction Formula 12:

Such diaphorase genes can include Bacillus spp., for example Bacillus cereus ATCC 14579 [Nature 423:87-91 (2003)], and the like.

In particular, enzymes derived from Thermus thermophilus, Thermotoga maritime, Sulfolobus tokodaii, Pyrobaculum aerophilum and the like has thermal resistance, and can be suitably used for the present invention.

Malic dehydrogenase activity is recognized as malic dehydrogenase for the fusion protein, and any known basic sequence of the genetic DNA or any known amino acid sequence of the enzyme can be used. That is to say, its derivation is not especially limited. Malic dehydrogenase is classified as follows.

(A) EC 1.1.1.37; (S)-malate:NAD+oxidoreductase

(B) EC 1.1.1.38; (S)-malate:NAD+oxidoreductase (oxaloacetate-decarboxylating)

(C) EC 1.1.1.39; (S)-malate:NAD+oxidoreductase (decarboxylating)

(D) EC 1.1.1.40; (S)-malate:NADP+oxidoreductase (oxaloacetate-decarboxylating)

Among them, balance of a chemical reaction of the enzyme EC 1.1.1.37 is biased to a malic acid production side, so that the enzymes classified into EC 1.1.38, EC 1.1.1.39 and EC 1.1.1.40 are more desirable. Such malic dehydrogenase can include Bacillus spp., for example Bacillus cereus ATCC 10987 [Nucleic Acids Res 32:977-88 (2004)], Bacillus subtilis 168 [Nature 390:249-56 (1997)], Lactobacillus spp., for example Lactobacillus plantarum WCFS1 [Proc Natl Acad Sci U.S.A. 100:1990-5 (2003)], Pseudomonas spp., for example Pseudomonas aeruginosa PA01 [Nature 406:959-64 (2000)], Pseudomonas putida KT2440 [Environ Microbiol 4:799-808 (2002)], Pyrococcus furiosus DSM 3638 [Genetics 152:1299-305 (1999), Mol Microbiol 38:684-93 (2000), Methods Enzymol 330:134-57 (2001)], Saccharomyces spp., for example Saccharomyces cerevisiae S288C [Science 274:546-67 (1996), Proc Natl Acad Sci U.S.A. 92:3809-13 (1995), EMBO J. 13:5795-809 (1994), Nature 357:38-46 (1992), Nature 387:75-8 (1997), Nature 387:78-81 (1997), Nat Genet 10:261-8 (1995), Nature 387:81-4 (1997), Science 265:2077-82 (1994), Nature 387:84-7 (1997), EMBO J. 15:2031-49 (1996), Nature 369:371-8 (1994), Nature 387:87-90 (1997), Nature 387:90-3 (1997), Nature 387:93-8 (1997), Nature 387:98-102 (1997), Nature 387:103-5 (1997)], Sulfolobus spp., for example Sulfolobus solfataricus [Proc Natl Acad Sci U.S.A. 98:7835-40 (2001)], Sulfolobus tokodaii strain 7 [DNA Res 8:123-40 (2001)], Thermotoga spp., for example Thermotoga maritima MSB8 [Nature 399:323-9 (1999)], Thermus spp., for example Thermus thermophilus HB27 [Nat Biotechnol 22:547-53 (2004)], and the like. In particular enzymes derived from Thermus thermophilus, Thermotoga maritime, Pyrococcus furiosus, Sulfolobus tokodaii and the like has thermal resistance, and can be suitably used for the present invention.

As glutamic dehydrogenase for the fusion protein (EC 1.4.1.2, EC 1.4.1.3 and EC 1.4.1.4), a function for catalyzing the following chemical reaction is identified, and any known basic sequence of the gene or any known amino acid sequence of the enzyme can be used. That is to say, its derivation is not especially limited.
L-glutamate+H₂O+NAD(P)⁺=2-oxoglutarate+NH₃+NAD(P)H+H⁺ Reaction Formula 13:

Such glutamic dehydrogenase genes can include Bacillus spp., for example Bacillus clausii KSM-K16, Bacillus subtilis 168 [Nature 390:249-56 (1997)], Burkholderia spp., for example Burkholderia mallei ATCC 23344 [Proc Natl Acad Sci U.S.A. 101:14246-51 (2004)], Deinococcus spp., for example Deinococcus radiodurans R1 [Science 286:1571-7 (1999)], Geobacillus spp., for example Geobacillus kaustophilus HTA426 [Nucleic Acids Res 32:6292-303 (2004)], Lactobacillus spp., for example Lactobacillus plantarum WCFS1 [Proc Natl Acad Sci U.S.A. 100:1990-5 (2003)], Pyrococcus spp., for example Pyrococcus horikoshii OT3 [DNA Res 5:55-76 (1998)], Pseudomonas spp., for example Pseudomonas aeruginosa PA01 [Nature 406:959-64 (2000)], Pseudomonas putida KT2440 (Environ Microbiol 4:799-808 (2002)], Pyrococcus spp., for example Pyrococcus abyssi GE5, Pyrococcus furiosus DSM 3638 [Genetics 152:1299-305 (1999), Mol Microbiol 38:684-93 (2000), Methods Enzymol 330:134-57 (2001)], Sulfolobus spp., for example Sulfolobus solfataricus (Proc Natl Acad Sci U.S.A. 98:7835-40 (2001)], Sulfolobus tokodaii strain 7 [DNA Res 8:123-40 (2001)], Thermococcus spp., for example Thermococcus kodakaraensis KOD1 [Genome Res 15:352-63 (2005)], Thermotoga spp., for example Thermotoga maritima MSB8 [Nature 399:323-9 (1999)], Thermus spp., for example Thermus thermophilus HB27 [Nat Biotechnol 22:547-53 (2004)], Thermus thermophilus HB8, and the like. In particular, enzymes derived from Thermus thermophilus, Thermotoga maritime, Pyrococcus furiosus, Sulfolobus tokodaii and the like has thermal resistance, and can be suitably used for the present invention.

As isomerase for the fusion protein (xyloseisomerase) (EC 5.3.1.5), a function for catalyzing the following chemical reaction is identified, and any known basic sequence of the genetic DNA or any known amino acid sequence of the enzyme can be used. That is to say, its derivation is not especially limited.
D-xylose=D-xylulose Reaction Formula 14:

Such isomerase genes can include Escherichia spp., for example Escherichia coli K-12 MG1655 [Science 277:1453-74 (1997)], Escherichia coli K-12 W3110, Bacillus spp., for example Bacillus subtilis 168 [Nature 390:249-56 (1997)], Lactococcus spp., for example Lactococcus lactis subsp. lactis IL1403 [Genome Res 11:731-53 (2001)], Thermotoga spp., for example Thermotoga maritima MSB8 [Nature 399:323-9 (1999)], and the like. In particular the enzymes derived from Thermotoga maritima has thermal resistance, and can be suitably used for the present invention.

As described above, the basic sequence of DNA for coding an amino acid sequence of the fusion protein used in the present invention can be obtained by connecting a basic sequence for coding the amino acid sequence of the enzyme 2 to the upstream or downstream of the basic sequence for coding the amino acid sequence of the enzyme 1. In this connection form, each of the enzymes 1 and 2 may activity in the fusion protein to be expressed according to the connected basic sequence.

In the amino acid sequences of the fusion protein, a spacer sequence can be inserted between the amino acid sequence of the enzyme 1 and the amino acid sequence of the enzyme 2. It is preferable that the structure and length of the spacer sequence are selected according to following requirements.

(1) The enzymes 1 and 2 can take unique folding respectively, and their respective enzyme activities are surely maintained.

(2) Among the reaction products 1 among the enzyme reaction catalyzed by the enzyme 1, chemical species to be matrices of the enzyme reaction catalyzed by the enzyme 2 can reach the enzyme 2 by diffusion as quickly as possible.

The length of such a spacer sequence is preferably about 3 to 400 amino acids, and a spacer sequence is not especially limited as long as it has the above-mentioned properties. For example, such a sequence as SEQ ID NO:137 or that about 2 or more and 5 or less such sequence units are repeated can be included. Also, following types as described in Patrick (J. Mol. Biol. (1990) 211, 934-958).

Type I (STG Type):

A sequence comprising only serine (S), threonine (T) and/or glycine (G).

Type II:

A sequence comprising serine, threonine and/or glycine, as well as one of asparaginic acid (D), lysine (K), glutamine (Q), asparagine (N), alanine (A) or proline (P).

Type III (SEQ ID NO:138 Type):

A combined sequence comprising amino acid residue STG Types I and II.

(Type III (SEQ ID NO:138 Type))

A sequence of combined sequence Types I and II comprising amino acid residues of combined sequences Types I and II comprising amino acid residue STG Types I and II, and having a plurality of proline.

Type IV (Pro Type):

A sequence of Types I and II having a plurality of proline.

Type V:

A sequence comprising an arbitrary combination of amino acid residues contained in Type III and having at least 8 amino acids.

Other than the above-mentioned types, the sequences can further include SEQ ID NO:139, SEQ ID NO:140, SEQ ID NO:141, SEQ ID NO:142, SEQ ID NO:143, SEQ ID NO:144 and the like.

The spacer sequence is inserted between the basic sequence for coding the amino acid sequence of the enzyme 1 and the basic sequence for coding the amino acid sequence of the enzyme 2. At that time, it is inserted so that respective reading frames of the basic sequence for coding the amino acid sequence of the enzyme 1 and the basic sequence for coding the amino acid sequence of the enzyme 2 are not displaced. The spacer sequence can be inserted using a method well known in the art.

In the fusion protein used in the present invention, even when a functional unit of the enzyme activity of the enzyme 1 or the enzyme 2 is not single-chain polypeptide (monomer), an object of the present invention can be achieved by adopting any of below-described constitutions.

(1) When the functional unit of any one of the enzymes is homooligomer.

For example, a functional polypeptide constitution of the enzyme 1 is expressed as αₙ (n>1 (integral number)), and a functional polypeptide constitution of the enzyme 2 is expressed as β. To obtain the fusion protein (αₙ::β) with a target polypeptide constitution, a constitutive polypeptide (α) of the enzyme using homooligomer as a functional unit is coexpressed in a host cell in addition to the fusion polypeptide (α::β). Two polypeptides (α::β and α) to be coexpressed may be coded on the same plasmid or coded on different plasmids. However, when different plasmids are used, it is required that both of the plasmids do not have incompatibility. In this system, non-target proteins with the peptide constitutions such as αₙ and αₙ₋ₓ(α::β)ₓ (x is an integral number to satisfy n+1>x>0) can be produced in addition to the target fusion protein (αₙ::β). They can be fractionated and purified using a gel filtration technique and an ultrafiltration, for example, according to the difference of respective molecular masses. Also, the fusion protein with the constitution of αₙ₋ₓ(α::β)ₓ (x is an integral number to satisfy n+1>x>0) can be supplied to the enzyme electrode according to the present invention, when the enzyme activities of both of the enzyme 1 and the enzyme 2 are held.

(2) When the functional unit of any one of the enzymes is heterooligomer.

For example, a functional polypeptide constitution of the enzyme 1 is expressed as αₙα′ (α′ shall comprehensively express polypeptide chains other than α, and n is an integral number to satisfy n>0), and a functional polypeptide constitution of the enzyme 2 is expressed as β. To obtain the fusion protein (αₙα′::β) with a target polypeptide constitution, constitutive polypeptides (α and α′) of the enzyme using homooligomer as a functional unit are coexpressed in a host cell in addition to the fusion polypeptide (α::β). The polypeptide chains (α::β, α and α′) to be coexpressed may be coded on the same plasmid or coded on different plasmids. However, when different plasmids are used, it is required that both of the plasmids do not have incompatibility. In this system, non-target proteins with the peptide constitutions such as αₙ₋ₓα′ (α::β)ₓ (x is an integral number to satisfy n+1>x>0) and αₙα′ can be produced in addition to the target fusion protein (αₙα′::β). They can be fractionated and purified using a gel filtration technique and an ultrafiltration, for example, according to the difference of respective molecular masses. Also, the fusion protein with the constitution of αₙ₋ₓα′ (α::β)ₓ (x is an integral number to satisfy n+1>x>0) can be supplied to the enzyme electrode according to the present invention, when the enzyme activities of both of the enzyme 1 and the enzyme 2 are held. To restrain production of such fusion proteins, among the constitutive units of heterooligomer, a smallest number of the functional constitutions, namely to satisfy n=1 is desirably selected as α. When α to satisfy n=1 can be selected, only the polypeptide chains α::β and α′ have to be coexpressed.

(3) When the functional units of both of the enzymes are homooligomer.

For example, a functional polypeptide constitution of the enzyme 1 is expressed as αₙ (n>1 (integral number)), and a functional polypeptide constitution of the enzyme 2 is expressed as βₘ (m>1 (integral number)). To obtain the fusion protein (αₙ::β) with a target polypeptide constitution, constitutive polypeptides (α and β) of the enzyme using homooligomer as a functional unit is coexpressed in a host cell in addition to the fusion polypeptide (α::β). Three polypeptides (α::β, α and β) to be coexpressed may be coded on the same plasmid or coded on different plasmids. However, when different plasmids are used, it is required that all of the plasmids do not have incompatibility. In this system, non-target proteins with the peptide constitutions such as αₙ, βₘ and αₙ₋ₓ(α::β)ₓβₘ₋ₓ(min(n,m)>x>1) can be produced in addition to the target fusion protein (αₙ::βₘ). They can be fractionated and purified using a gel filtration technique and an ultrafiltration, for example, according to the difference of respective molecular masses. Also, the fusion protein with the constitution of αₙ₋ₓ(α::β)ₓβₘ₋ₓ (min(n,m)>x>1) can be supplied to the enzyme electrode according to the present invention, when the enzyme activities of both of the enzyme 1 and the enzyme 2 are held.

(4) When a functional unit of one of the enzymes is homooligomer and a functional unit of the other enzyme is heterooligomer.

For example, a functional polypeptide constitution of the enzyme 1 is expressed as αₙ (n>1 (integral number), and a functional polypeptide constitution of the enzyme 2 is expressed as βₘβ′ (β′ shall comprehensively express polypeptide chains other than β, and m is an integral number to satisfy m>0). To obtain the fusion protein (αₙ::βₘβ′) with a target polypeptide constitution, constitutive polypeptides (α, β and β′) of the enzyme using homooligomer as a functional unit are coexpressed in a host cell in addition to the fusion polypeptide (α::β). The polypeptide chains (α::β, α and β′) to be coexpressed may be coded on the same plasmid or coded on different plasmids. However, when different plasmids are used, it is required that both of the plasmids do not have incompatibility. In this system, non-target proteins with the peptide constitutions such as αₙ, β_{mβ′, and α}ₙ-ₓ(α::β)ₓβₘ₋ₓβ′ (min(n,m)>x>0) can be produced in addition to the target fusion protein (αₙ::βₘβ′). They can be fractionated and purified using a gel filtration technique and an ultrafiltration, for example, according to the difference of respective molecular masses. Also, the fusion protein with the constitution of αₙ-x(α::β)ₓβₘ₋ₓβ′ (min(n,m)>x>0) can be supplied to the enzyme electrode according to the present invention, when the enzyme activities of both of the enzyme 1 and the enzyme 2 are held. To restrain production of such fusion proteins, among the constitutive units of heterooligomer, a smallest number of the functional constitutions, namely to satisfy m=1 is desirably selected as β. When β to satisfy m=1 can be selected, only the polypeptide chains α::β, α and β′ have to be coexpressed.

(5) When the functional activities of both of the enzymes are heterooligomer.

For example, a functional polypeptide constitution of the enzyme 1 is expressed as αₙα′ (α′ shall comprehensively express polypeptide chains other than α, and n is an integral number to satisfy n>0), and a functional polypeptide constitution of the enzyme 2 is expressed as βₘβ′ (β′ shall comprehensively express polypeptide chains other than β, and m is an integral number to satisfy m>0). To obtain the fusion protein (αₙα′::βₘβ′) with a target polypeptide constitution, constitutive polypeptides (α, α′, β and β′) of the enzyme using homooligomer as a functional unit are coexpressed in a host cell in addition to the fusion polypeptide (α::β). The polypeptide chains (α::β, α, α′, β and β′) to be coexpressed may be coded on the same plasmid or coded on different plasmids. However, when different plasmids are used, it is required that both of the plasmids do not have incompatibility. In this system, non-target proteins with the peptide constitutions such as αₙα′, βₘβ′, and αₙ₋ₓα′ (α::β)ₓβₘ₋ₓβ′ (min(n,m)>x>0) can be produced in addition to the target fusion protein (αₙα′::βₘβ′). They can be fractionated and purified using a gel filtration technique and an ultrafiltration, for example, according to the difference of respective molecular masses. Also, the fusion protein with the constitution of αₙ-ₓα′ (α::β)ₓβₘ₋ₓβ′ (min(n,m)>x>0) can be supplied to the enzyme electrode according to the present invention, when the enzyme activities of both of the enzyme 1 and the enzyme 2 are held. To restrain production of such fusion proteins, it is desirable that among the constitutive units of heterooligomer, a smallest number of the functional constitutions, namely to satisfy n=1 is selected as α and a smallest number of the functional constitutions, namely to satisfy m=1 is selected as β. When a to satisfy n=1 and β to satisfy m=1 can be selected, only the polypeptide chains α::β, α′ and β have to be coexpressed.

Additionally, in all of the above-mentioned cases (1) to (5), constitutions of expression with the enzyme 1 and the enzyme 2 inverted are also possible.

In all of the above-mentioned cases (1) to (5), not an intracellular coexpression system, but an in vitro acellular protein synthesizing system can be adopted. In this case, after respective constitutive polypeptides are synthesized by an in vitro protein synthesizing device, they are mixed, so that the fusion protein having a target polypeptide constitution can be formed.

By inserting DNA for coding the amino acid sequence of the fusion protein used for the enzyme electrode according to the present invention to the downstream of a promoter of an appropriate expression vector, a fusion protein expression vector expressable in various hosts can be constructed. A person skilled in the art can construct a functional protein expression vector can be constructed in an arbitrary host cell according to a normal method in the art. The promoter may be selected from known ones, or newly prepared. An expression vector capable of producing the fusion protein at high level can be constructed by modification (for example, exchange of promoter) using a normal skill.

The host cell used for genetic transformation with a fusion protein expression vector may be any of a prokaryotic cell such as E. coli and a eukaryotic cell such as yeast, and furthermore may be a cell of higher organisms generally available. The host cell can include microorganisms (prokaryotic organism (bacteria such as E. coli and Bacillus subtilis) and eukaryotic organisms (such as yeast)], animal cells, or cultured plant cells. Prokaryotic organisms and yeast are preferable as microorganisms. Strains especially belonging to Escherichia spp. (for example, such as E. coli) are preferable as prokaryotic organisms. Strains especially belonging to Saccharomycess spp. (for example, S. cerevisiae) and Candida spp. (for example, C. boidinii) are preferable as yeast. Animal cell strains can include, for example, cells of mouse L929, cells of Chinese hamster ovarian (CHO), and the like.

An expression vector suitable for using microorganism, in particular E. coli as a host cell is already known. For example, the expression vector can have a conventional promoter such as a lac promoter and a tac promoter. It is preferable the expression vector for expressing the fusion protein by yeast contains a promoter such as a GAL promoter and an AOD promoter. Also, the expression vector for expressing the fusion protein by mammal cells has a promoter such as an SV40 promoter. However, in view of easiness of operations and procurements, a prokaryotic host is preferable as a host cell, and E. coli is more preferable. A prokaryotic host-vector system is described in many publications, and already known in the art, but will be simply explained as follows. (For example, see Molecular Cloning: A LABORATORY MANUAL, Cold Spring Harbor Laboratory Press.)

To express DNA for coding the fusion protein with E. coli, the DNA is inserted on the downstream of a promoter of a plasmid suitable for genetic transformation using E. coli.

One aspect of expression with E. coli will be described in below-described examples.

However, in expression in other aspects, for example DNA for coding the fusion protein is treated with an appropriate enzyme, so as to obtain a DNA fragment for coding the fusion protein. Then, the fragment is embedded in an appropriate vector, so that peptide having the fusion protein activity can be expressed in various hosts. Here, an appropriate enzyme means, for example, a restriction enzyme, alkaline phosphatase, polynucleotide kinase, DNA ligase, DNA polymerase and the like.

A method for genetically transforming a host cell with a vector for expressing protein is already known, and the fusion protein used in the present invention can be expressed using this known method. For example, the fusion protein can be expressed by a competent cell production method in the case of a prokaryotic host, by a competent cell production method in the case of a eukaryotic host, and by a transfection method and an electroporation in the case of a mammal cell. Then, the resultant transformant is cultured in an appropriate culture medium. The culture medium can contain carbon sources (for example, such as glucose, methanol, galactose and fructose), and inorganic or organic sources (for example, such as ammonium sulfate, ammonium chloride, sodium nitrate, peptone, and casamino acid). If desired, other nutrition sources (for example, inorganic salts (such as sodium chloride and potassium chloride), vitamins (for example, vitamin B1), and antibiotics (for example, such as ampicillin, tetracycline and kanamycin) may be added to the culture medium. An eagle culture medium is suitable for culturing the mammal cells.

The transformant may be cultured for 8 to 48 hours normally at pH 6.0 to 8.0, preferably pH 7.0 and at 25 to 40° C., preferably 30 to 37° C. When the produced fusion protein exists in a culture solution, the cultured substance is filtrated or centrifuged. The cultured substance can be purified using a normal method used for purifying and isolating natural or synthetic proteins from a supernatant of the collected culture solution. For example, dialysis, gel filtration, affinity column chromatography using a corresponding anti-fusion protein monoclonal antibody, column chromatography using an appropriate adsorbent, high-speed liquid chromatography and the like can be used. When the produced fusion protein exists in a periplasm and a cytoplasm of the cultured transformant, the cells are collected by filtration or centrifugation, and their cell walls and/or cell membranes are broken, for example, by a ultrasonic and/or lysozyme treatment, so as to obtain cytocidal substances. An appropriate aqueous solution (for example, buffer) is mixed with the cytocidal substances, so that the fusion protein can be purified by a normal method. When it is necessary to regenerate (refold) the produced fusion protein in E. coli, it can be regenerated by a normal method. In particular, when the fusion enzymes derived from thermophile are prepared, proteins derived from host room-temperature microorganisms are aggregated by constantly holding the cyptocidal liquid at the temperature of 70° C. or more. The aggregates are removed by a centrifugal operation, so that it can be simply purified. Also, it can be transformed into a fold having activity.

Also, the fusion protein does not have to be completely purified according to usage, and may be any of following items (1) to (6).

(1) Living cell: a cell which is separated from a cultured substance by a normal method such as filtration or centrifugation.

(2) Dry cell: a living cell described in Item (1), which is freeze-dried or vacuum-dried.

(3) Cell extract: a cell described in Item (1) or (2), which is treated by a normal method (for example, self-bacteriolysis in an organic solvent, trituration by mixture with alumina and sea sands, or ultrasonic treatment).

(4) Enzyme solution: the cell extract described in Item (3), which is normally or partially purified.

(5) Purified enzyme: the enzyme solution described in Item (4), which is further purified not to contain impurities.

(6) Fragment having enzyme activity: a peptide fragment which is obtained by fractionating the purified enzyme described in Item (5) by an appropriate method.

The fusion protein used for the enzyme electrode according to the present invention can be mostly acquired as a holoenzyme bonded with a prosthetic group such as a flavine compound, a metal atom (such as Fe, Cu and Mo) and hem. In particular, by adding these prosthetic groups in the culture medium in advance at the time of biosynthesis, a recovery rate of the holoenzymes can be increased. However, when the fusion peptides are acquired in vitro using an acellular protein synthesizing device, the protein is acquired as an apoenzyme, to which the prosthetic group is not bounded. In this case, a step for holding the acquired apoenzymes in a buffer, to which the prosthetic groups are added, is added, so that holoenzymes can be reconstructed.

The corresponding protein with mutation introduced in the amino acid sequence of the fusion protein can be produced by a normal method genetically used on the basis of a genetic basic sequence of the fusion protein used for the enzyme electrode according to the present invention. This mutation can include substitution, deletion, insertion, transfer or addition of one or more amino acids. Such mutation, transformation and modification methods are described in following documents.

The Japanese Biochemical Society ed., “Zoku Seikagaku Jikken Kouza 1, Idenshi Kenkyu-hou II (Follow-up Biolchemical Experiment Course 1, Genetic Study II)”, p. 105 (Susumu Hirose), Tokyo Kagaku Dozin Co., Ltd. (1986): The Japanese Biochemical Society ed., “Shin Seikagaku Jikken Kouza 2, Kakusan III, Kumikae DNA Gijutsu (New Biochemical Experiment Course 2, Nucleic Acid III, Recombinant DNA Technique)”, p. 233 (Susumu Hirose), Tokyo Kagaku Dozin Co., Ltd. (1992); R. Wu, L. Grossman, ed., “Methods in Enzymology”, Vol. 154, p. 350 & p. 367, Academic Press, New York (1987); R. Wu, L. Grossman, ed., “Methods in Enzymology”, Vol. 100, p. 457 & p. 468, Academic Press, New York (1983); J. A. Wells et al., Gene, 34: 315, 1985; T. Grundstroem et al., Nucleic Acids Res., 13: 3305, 1985; J. Taylor et al., Nucleic Acids Res., 13: 8765, 1985; R. Wu ed., “Methods in Enzymology”, Vol. 155, p. 568, Academic Press, New York (1987); and A. R. Oliphant et al., Gene, 44: 177, 1986.

For example, the methods include a position-identifying mutation introducing method (a site-specific mutation introducing method) using synthetic oligonucleotide, a Kunkel method, dNTP[αS] method (Eckstein), a region-identifying mutation introducing method using sulfurous acid and nitrous acid, and the like.

Furthermore, amino acid residues contained in the resultant fusion protein can be modified by a chemical manner. Furthermore, the fusion protein can be modified or partially decomposed into its derivative using enzymes such as peptidase, for example, pepsin, chymotrypsin, papain, bromelain, endopeptidase and exopepetidase.

Also, the protein may be expressed as the fusion protein fused with tags for purification or a transition signal sequence. Such fusion protein fused with tags for purification or a transition signal sequence can be produced using a fusion producing method normally used in a genetic engineering. The fusion protein fused with the tags for purification can be purified by an affinity chromatography using its tag parts for purification, or can be used as an immobilizing tag for forming the above-mentioned enzyme immobilized layer.

That is to say, in the fusion protein of the enzyme electrode according to the present invention, one or more amino acid residues may be different from natural amino acid residues of the enzymes 1 and 2 in terms of identity, and the position of one or more amino acid residues may be different from that of the natural amino acid residues. The fusion protein may be a deletion analogue, wherein one or more (for example, such as 1 to 80, preferably 1 to 60, more preferably 1 to 40, much more preferably 1 to 20, and in particular 1 to 10) amino acid residues unique to natural protein of the enzymes 1 and 2 are deleted. Also, the fusion protein may be a substitution analogue, wherein one or more amino acid residues unique to natural protein of the enzymes 1 and 2 are substituted by other residues. In this case, a number of the substituted amino acid residues may be 1 to 80, preferably 1 to 60, more preferably 1 to 40, much more preferably 1 to 20, and in particular 1 to 10.

The fusion protein may be an addition analogue, wherein one or more amino acid residues unique to natural protein of the enzymes 1 and 2 are added. In this case, a number of the added amino acid residues may be 1 to 80, preferably 1 to 60, more preferably 1 to 40, much more preferably 1 to 20, and in particular 1 to 10. These mutations can include those having maintained domain structures which are characteristics of the natural protein of the enzymes 1 and 2. Also, these mutations can include those having primary structural conformations substantially equivalent to the natural protein of the enzymes 1 and 2, or one part thereof. Furthermore, these mutations can include those having biological activity substantially equivalent to that of the natural protein of the enzymes 1 and 2. The above-mentioned mutants can be all used as the fusion protein of the enzyme electrode according to the present invention.

The mutant fusion protein includes the protein, which has high homology to amino acid sequences expressed by SEQ ID NO:11, 23, 35, 44, 57, 69, 79, 88, 97, 106, 114, 121, 128 or 135 in a sequence list, and whose activities of the enzymes 1 and 2 are maintained. Such protein includes those having homogenous amino acid sequences having homology of 70% or more, more preferably 80% or more, and most preferably 90% or more.

The mutant fusion protein translated from the DNA consisting of a basic sequence complementary to the DNA including a basic sequence for coding the amino acid sequence of the natural protein of the enzymes 1 and 2, and the mutant DNA hybridized under a stringent condition can be also used in the present invention. The basic sequences as a reference of the mutation under this stringent condition include basic sequences expressed by SEQ ID NO:10, 22, 34, 43, 56, 68, 78, 87, 96, 105, 113, 120, 127 and 134. Also, the mutation in a range to be hybridized under this stringent condition does not deteriorate the activities of the enzymes 1 and 2.

Furthermore, the following mutant fusion protein having enzyme activities substantially equivalent to those of the enzymes 1 and 2 can be used as the fusion protein of the enzyme electrode according to the present invention.

Enzyme 1:

SEQ ID NO:10, 22, 127 and 134: glucose dehydrogenase

SEQ ID NO:34, 43, 113 and 120: alcohol dehydrogenase

SEQ ID NO:56 and 68: lactic dehydrogenase

SEQ ID NO:78 and 87: malic dehydrogenase

SEQ ID NO:96 and 105: glutamic dehydrogenase

Enzyme 2:

SEQ ID NO:10, 22, 34, 43, 56, 68, 78, 87, 96 and 105: diaphorase

SEQ ID NO:113 and 120: aldehyde dehydrogenase

SEQ ID NO:127 and 134: isomerase

Herein, the above-mentioned DNA “hybridized under a stringent condition” refers to following DNA.

(1) By hybridization under a temperature condition of 65° C. in a high ion concentration, the DNA consisting of sequences complementary to the above-mentioned basic sequences as a reference, and the DNA consisting of basic sequences including mutation form DNA-DNA hybrid.

(2) The hybrid achieved in the above-mentioned item (1) is maintained even after it is cleansed for 30 minutes under a temperature condition of 65° C. in a low ion concentration.

Also, the high ion concentration condition in the above-mentioned item (1) can be achieved by 6×SSC (900 mN sodium chloride and 90 mN sodium citrate). The low ion concentration condition in the above-mentioned item (2) can be achieved by 0.1×SCC (15 mN sodium chloride and 1.5 mN sodium citrate).

Concretely, the DNA is comprised of basic sequences, wherein one or several amino acid residues are deleted, substituted or added to basic sequences expressed by SEQ ID NO:10, 22, 34, 43, 56, 68, 78, 87, 96, 105, 113, 120, 127 or 134. This mutation is done within a range not to deteriorate the activities of the enzymes 1 and 2.

Herein, “substantially equivalent” to the natural protein of the enzymes 1 and 2 means that activities of the protein, for example catalytic activity, physiological activity and biological activity are substantially identical.

Substitution, deletion or insertion of the amino acids often does not cause a big change to a physiological characteristic or a chemical characteristic of polypeptide. In this case, such amino-acid substituted, deleted or inserted polypeptide is substantially identical to polypeptide not substituted, deleted or inserted. A substantially identical substituent of the amino acid in the amino acid sequence can be selected from other amino acids in classes, to which the amino acid belong. For example, a nonpolar (hydrophobic) amino acid includes alanine, phenylalanine, leucine, isoleucine, valine, proline, tryptophan, methionine and the like. A polar (neutral) amino acid includes glycine, serine, threonine, cysteine, tyrosine, asparagines, glutamine and the like. A positive electric amino acid (basic amino acid) includes arginine, lysine, histidine and the like, and a negative electric amino acid (acidic amino acid) includes asparaginic acid, glutamic acid and the like.

An electron transfer mediator of the enzyme electrode according to the present invention is a substance which is contained in an electrode system to promote an electron reception reaction between the enzyme and the conductive base plate and heighten measuring sensitivity and current density. This electrode mediator is not especially limited, as long as it is the above-mentioned substance. For example, the substance includes a metal complex consisting of central metal and its ligand or its ionized material, metallocenes, phenazine methosulfate, 1-methoxy-phenazine methosulfate, quinines, phenazines, phenothiazine, viologen, benzylviologen, 2,6-dichlorophenol-indophenol (DCIP), methylene blue, meldola blue, toluidine blue, gallocyanine, thionine, thionine dimethylsulfoxide, new methylene blue, brilliant cresyl blue, gallocyanine, resorufin, alizarine brilliant blue, safranine, their derivatives and the like.

The metal complex compound includes at least one kind selected from Os, Fe, Ru, Co, Cu, Ni, V, Mo, Cr, Mn, Pt, Ph, Pd, Mg, Ca, Sr, Ba, Ti, Ir, Zn, Cd, Hg and W as central metal. The ligand to the central metal includes pyrrole, pyrazole, imidazole, 1,2,3- or 1,2,4-triazole, tetrazole, 2,2′-biimidazole, pyridine, 2,2′-bithiophene, 2,2′-bipyridine, 2,2′:6′2″-terpyridine, ethylenediamine, porphyrin, phthalocyanine, acetylacetone, xylinol, ammonia, cyanogen ion, triphenylphosphine oxide, cyclopentadienyl ring and their derivatives.

Concretely, the metal complex and its ionized material can include a bipyridine complex formed of metal such as osmium, ruthenium, cobalt and nickel, and bipyridine, and a metal complex ion such as ferricyanic ion, octacyano tungsten acid ion and octacyano molybdenum acid ion.

Metallocenes can include, for example, ferrocene derivatives such as ferrocene, 1,1′-dimethyl ferrocene, ferrocene carboxylate and ferrocene carboxyaldehyde.

Two or more kinds of these electron mediators can be combined within a range not to deteriorate advantages of the present invention.

The electron mediators are contained in the enzyme electrode according to the present invention in an amount of 0.5 to 10 wt %, preferably 1 to 5 wt % of the entire components. Herein, the entire components mean components contained in the enzyme immobilized layer on the conductive base plate.

Enzyme electrode devices (biosensor, fuel cell and electrochemical reaction device) which can be used for various usages by connecting wires for receiving and giving electrons can be manufactured to the enzyme electrode according to the present invention. The devices can be constituted by using the plate-like (or film-like or layer-like) enzyme electrode in a single layer or a plurality of the electrodes. When a plurality of the electrodes are used, the electrodes can be laminated in a manner that a surface of one electrode is opposed to the rear surface of the other. Also, when a plurality of the electrodes are used, respective enzyme electrodes may have constant characteristic, or different characteristics in combination. For example, like a below-described fuel cell, anodes and cathodes can be alternatively arranged. The devices can cope with required voltage and output by changing a number of stages of the electrode from a single layer to multiple layers. The enzyme as a catalyst of the enzyme electrode has substrate selectivity higher than that of a noble metal catalyst (for example, platinum) generally used in an electrochemical field. Therefore, there is no need of a mechanism for isolating a reaction substance of one electrode from the reaction substance of the other electrode. As a result, the devices can be simplified.

The sensor in one preferred embodiment of the present invention has a constitution that the enzyme electrode according to the present invention is used as a detection portion for detecting substances. A representative constitution can include such a constitution that the enzyme electrode used as a reaction electrode in set with a reference electrode and a counter electrode and detects a detectable current (by a function of the enzyme immobilized to the electrode). By detecting existence and a volume of the current, existence and a volume of the substances in a liquid, with which these electrodes come into contact, can be detected. Concretely, the sensor with the constitution shown in FIG. 8 can be shown. The sensor shown in FIG. 8 has a reaction electrode 4, a platinum wire counter electrode 5, and a silver chloride reference electrode 6, and lead wires 7, 8 and 9 are wired to the respective electrodes and connected with a potentiostat 10. The sensor is arranged in a storage area of a sample solution 3 in a water jacket cell 1 sealable with a cover 2. By measuring a steady-state current, upon application of a potential to the reaction electrode, a substrate in an electrolyte can be detected. Also, when measurement in an inactive gas atmosphere is required, inactive gas such as nitrogen is introduced from a gas blow port 11 at an external terminal of a gas tube 12. The temperature can be controlled by supplying liquid for temperature control utilizing a temperature control water flow inlet 13 and a temperature control water exhaust port 14.

The fuel cell in one preferred embodiment of the present invention uses the enzyme electrode as at least one of an anode and a cathode. In this case, the enzyme electrode can be made into a plate-like or layer-like form, and used in a single layer or in a laminated structure of two or more layers. Furthermore, when the laminated structure is adopted, the anode and the cathode may be located in a predetermined alignment in a lamination direction. The representative constitution can include such a constitution that the fuel cell has a reaction vessel capable of storing electrolyte containing a substance to be fuel, and an anode and a cathode arranged in the reaction vessel at a predetermined interval, and the enzyme electrode according to the present invention is used for at least one of the anode and the cathode. Also, the fuel cell may be of a type for complementing or circulating the electrolyte, or a type not for complementing or circulating the electrolyte. A kind, a structure and a function of the fuel cell are note limited, as long as the enzyme electrode can be used in the fuel cell. At that time, the enzyme used as a catalyst of the enzyme electrode has high substrate selectivity, so that there is no need of a mechanism for isolating a reaction substance of one electrode from the reaction substance of the other electrode. As a result, the devices can be simplified. The fuel cell can oxidize and reduce substances at low overvoltage by a high catalytic reaction unique to the enzyme used as a catalyst of the electrode reaction, so that a high driving voltage can be obtained, and a long life and high output can be realized by high stability and current density.

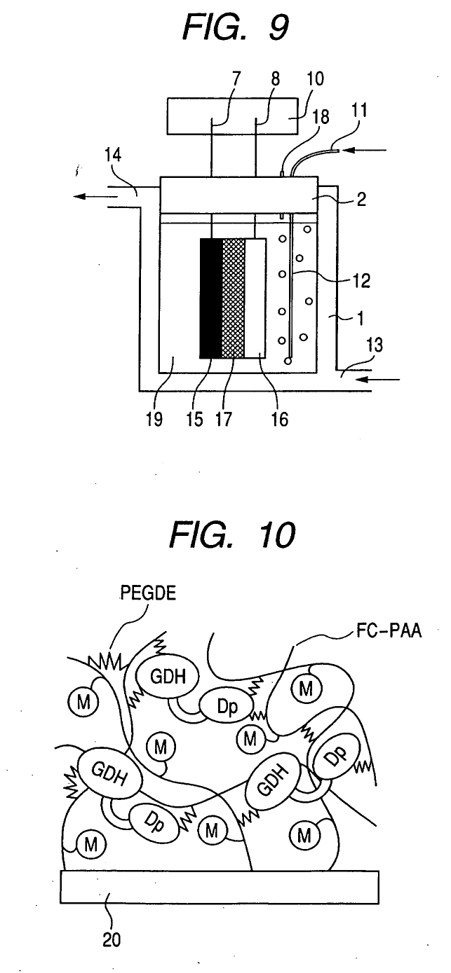

One example of the fuel cell is shown in FIG. 9. The constitution of cells of the fuel cell is approximately identical to those of a substrate measuring device of the sensor previously shown in FIG. 8. Identical numbers are added to identical members. Instead of the sensor shown in FIG. 8, an electrode unit with a constitution that an anode 15 and a cathode 16 are laminated via a porous polypropylene film 17 is used, and oxygen gas is introduced in the cell via the tube 12, so that it is made to act as a fuel cell.

The electrochemical reaction device in one preferred embodiment of the present invention can have high substrate selectivity and catalytic ability unique to the enzyme used as a catalyst of an electrode reaction. Additionally, quantitative property of a response which is a characteristic of the electrochemical reaction can be further obtained. As a result, the device can be quantitatively controlled at high selectivity and high efficiency, and a long life and high output of the device can be realized at high stability and current density of the enzyme electrode using a carrier and a plurality of mediators. In this case, the enzyme electrode can be made into a plate-like or layer-like form, and used in a single layer or in a laminated structure of two or more layers. A representative constitution can include such a constitution that a pair of electrodes and a reference electrode installed if necessary are arranged in a reaction vessel capable of storing reaction liquid and current is conducted between a pair of the electrodes, so that an electrochemical reaction is caused in substances in the reaction liquid to obtain a target reaction product, resolvent or the like. In this case, the enzyme electrode according to the present invention can be used for at least one of a pair of the electrodes. The constitutions of the device such as kinds of the reaction liquid and condition of the reaction are not especially limited, as long as the enzyme electrode can be used. For example, the device can be used to acquire the reaction product by an oxidation-reduction reaction, or acquire a target resolvent.

Also, such an example is known that it is effective to use the fusion protein with following enzymes 1 and 2 fused for catalyzing two associated reactions for a reaction system comprising plural kinds of enzymes, for improving reaction efficiency and reaction speed.

Enzyme 1: Catalyzing a chemical reaction for producing a reaction product 1 from a reaction substrate 1.

Enzyme 2: Catalyzing a chemical reaction for producing a reaction product 2 from a reaction substrate 2.

Seo et al. (Applied and Environmental Microbiology 66(6) 2000, 2484-2490) discloses influences of fusion of enzymes on a reaction for synthesizing trehalose from UDP-glucose and glucose-6-phosphate via trehalose-6-phosphate. Here, the fusion protein of trehalose-6-phosphate synthetase and trehalose-6-phosphate phosphatase is used. When this fusion protein is used, the reaction speed is improved 3.5 to 4.0 times faster than that when the respective enzymes are made to function in an isolated condition.

However, in the fusion protein known in the above-mentioned document, electron reception to the electrodes is not recognized, so that it cannot be used as an enzyme electrode.

Therefore, the present inventors tried to produce a new fusion protein as described above, and the present invention was made.

The present invention will be explained in detail, but a method of the present invention is not limited to these examples. Example Nos. 14, 15, 16, 22, 23, 24, 30, 31, 32, 38, 39, 40, 46, 47 and 48 are intentionally missing.

### EXAMPLE 1

### Preparation of Fusion Protein (His-busGDH::ppuDp) (SEQ ID NO:10 and 11) of Glucose Dehydrogenase (busGDH) Derived from Bacillus subtilis and Diaphorase (ppuDp) Derived from Pseudomonas putida

Genomic DNA is prepared from Bacillus subtilis by a normal method. The genomic DNA is used as a mold and following synthetic oligo DNA is used as a primersynthetic oligo DNAs SEQ ID NO:1 having a sequence recognized by BamHI and SEQ ID NO₂: having a sequence recognized by HindIII are used as primers for performing PCR, so as to obtain a DNA amplified product of 805 bp.

The DNA amplified product is cut by restriction enzymes BamHI and HindIII, and inserted in the same restriction enzyme site of pETDuet-1 (manufactured by Novagen) so as to produce a His-busGDH expression vector pETDuet-busGDH.

Then, genomic DNA is prepared from Pseudomonas putida KT2440 (ATCC 47054) by a normal method. The genomic DNA is used as a mold and following synthetic oligo DNA is used as a primersynthetic oligo DNAs SEQ ID NO:3 having a sequence recognized by NdeI and SEQ ID NO:4 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pETDuet-busGDH so as to produce a His-busGDH, ppuDp coexpression vector pETDuet-busGDH-ppuDp.

Then, following 5′ end phosphorylated synthetic oligo DNA is equivalent mixed in a TE buffer, and heated, and then annealed by gradual cooling.

SEQ ID NO:5

SEQ ID NO:6

The DNA fragment is inserted in a recognition site of the restriction enzymes HindIII and NdeI of pETDuet-busGDH-ppuDp so as to produce a fusion protein His-busGDH::ppuDp expression vector pETDuet-busGDH::ppuDp (SEQ ID NO:7) wherein Hiswherein His-busGDH and ppuDp are bounded by a spacer sequence SEQ ID NO:145.

Then, the genomic DNA of Bacillus subtilis (ATCC 27370) is used as a mold and following synthetic oligo DNA is used as a primersynthetic oligo DNAs SEQ ID NO:8 having a sequence recognized by NcoI and SEQ ID NO:2 having a sequence recognized by HindIII are used as primers for performing PCR, so as to obtain a DNA amplified product of 805 bp.

The DNA amplified product is cut by restriction enzymes NcoI and HindIII, and inserted in the same restriction enzyme site of pCDFDuet-1 (manufactured by Novagen) so as to produce a busGDH expression vector pCDFDuet-busGDH.

Then, the genomic DNA of Pseudomonas putida KT2440 (ATCC 47054) is used as a mold and following synthetic oligo DNA is used as a primersynthetic oligo DNAs SEQ ID NO:3 having a sequence recognized by NdeI and SEQ ID NO:4 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pCDFDuet-busGDH so as to produce a busGDH, ppuDp coexpression vector pCDFDuet-busGDH-ppuDp (SEQ ID NO:9)

E. coli BL21 (DE3) is transformed by the expression vectors pETDuet-busGDH::ppuDp and pCDFDuet-busGDH-ppuDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### COMPARISON EXAMPLE 1

### Preparation of busGDH and ppuDp as a Control

Genomic DNA is prepared from Bacillus subtilis (ATCC 27370) by a normal method. The genomic DNA is used as a mold and following synthetic oligo DNA is used as a primersynthetic oligo DNAs SEQ ID NO:12 having a sequence recognized by NdeI and SEQ ID NO:13 having a sequence recognized by XhoI are used as primers for performing PCR, so as to obtain a DNA amplified product of about 800 bp.

The DNA amplified product is cut by restriction enzymes NdeI and XhoI, and inserted in the same restriction enzyme site of pET-21a(+) (manufactured by Novagen) so as to produce a busGDH-His expression vector pET21-busGDH.

Then, genomic DNA is prepared from Pseudomonas putida KT2440 (ATCC 47054) by a normal method. The genomic DNA is used as a mold and following synthetic oligo DNA is used as a primersynthetic oligo DNAs SEQ ID NO:3 having a sequence recognized by NdeI and SEQ ID NO:14 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pET-21a(+) (manufactured by Novagen) so as to produce a ppuGDH-His expression vector pET21-ppuDp.

E. coli BL21 (DE3) is transformed according to a normal method by independently using the expression vectors pET21-busGDH and pET21-ppuDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion proteins are purified using a nickel chelate column.

### EXAMPLE 2

### Glucose Sensor

Example 2 will be explained with reference to FIG. 10 and FIG. 8. The sensor in Example 2 is a glucose sensor for determining the quantity of glucose in a sample solution. The enzyme electrode part of the glucose sensor in Example 2 will be explained with reference to FIG. 10. The enzyme electrode part is constituted as follows.

A conductive base plate 20 is glassy carbon with diameter of 3 mm. The fusion protein (His-busGDH::ppuDp), and ferrocene-bounded polyallylamine (Fc-PAA) are crosslinked and immobilized on the conductive base plate 20 by poly(ethylene glycol) diglycigyl ether. The fusion protein is the fusion protein of glucose dehydrogenase (busGDH) derived from Bacillus subtilis and diaphorase (ppDp) derived from Pseudomonas putida prepared in Example 1. Hereinafter, poly(ethylene glycol) diglycigyl ether is abbreviated as PEGDE.

Each of the components is immobilized at following optimal concentrations, respectively. That is to say, the components are prepared at His-busGDH::ppuDp (busGDH: 0.3 unit and ppuDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute a glucose sensor. The counter electrode 5 is formed of a platinum wire, and the reference electrode 6 is made of a silver/silver chloride electrode. These electrodes measure current and the like via a potentiostat 10, respectively. 0.1 M PIPES-NaOH buffer aqueous solution (pH 7.5) containing a predetermined concentration of glucose and 1 mM NAD is used for the sample solution 3. The measured temperature is held at 37° C. by a constant temperature circulation vessel.

The quantity is determined using the above-mentioned glucose sensor as follows. A potential of 300 mV is applied to the reference electrode 6 in the reaction electrode 4. At that time, glucose in the sample solution 3 is oxidized to gluconolactone in the presence of glucose dehydrogenase, and NAD is reduced to NADH by this reaction. Then, NADH is oxidized to NAD in the presence of diaphorase fused to the glucose dehydrogenase. Ferrocene as an electron transfer mediator is oxidized by this reaction so as to produce a ferricinium ions. The ferricinium ions receive electrons from the reaction electrode 4 and are reduced to ferrocene (because the potential of 300 mV is applied to the reference electrode 6 in the reaction electrode 4). The current by movement of the electrons at the reaction electrode 4 is measured, so as to measure the concentration of glucose in the sample solution.

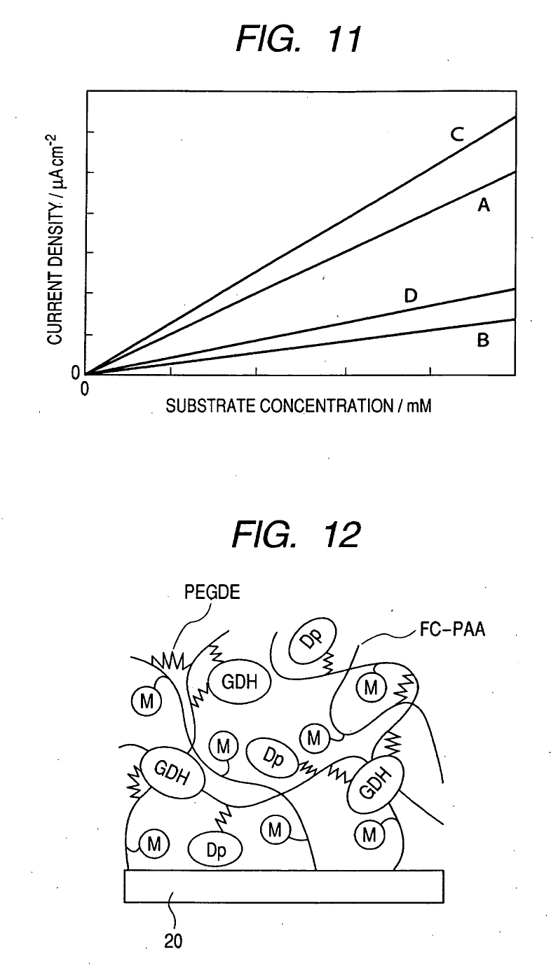

One example of a trend of measured results in Example 2 is shown in FIG. 11. A solid line A in FIG. 11 shows a relationship between a variation volume of the reducing current measured at the reaction electrode 4 and the glucose concentration. From FIG. 11, it is confirmed that the variation volume of the reducing current and the glucose concentration show a good proportional relationship. Therefore, the quantity of glucose can be precisely determined using the glucose sensor in Example 2.

### COMPARISON EXAMPLE 2

### Glucose Sensor

Comparison Example 2 will be explained with reference to FIG. 12 and FIG. 8. The sensor in Comparison Example 2 is a glucose sensor for determining the quantity of glucose in a sample solution. The enzyme electrode part of the glucose sensor in Comparison Example 2 will be explained with reference to FIG. 12.

The enzyme electrode part is constituted as follows. A conductive base plate 20 is glassy carbon with diameter of 3 mm. Glucose dehydrogenase (busGDH), diaphorase (ppDp), and ferrocene-bounded polyallylamine (Fc-PAA) are crosslinked and immobilized on the conductive base plate 20 by PEGDE. Glucose dehydrogenase (busGDH) is glucose dehydrogenase derived from Bacillus subtilis prepared from Comparison Example 1, and diaphorase (ppDp) is diaphorase derived from Pseudomonas putida. Each of the components is immobilized at following optimal concentrations, respectively. That is to say, the components are prepared at busGDH: 0.3 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute a glucose sensor. As is similar to Example 2, when measurement is done, a trend is shown in a solid line B.

From Example 2 and Comparison Example 2, it becomes apparent that the glucose sensor of Example 2 has higher sensitivity to the glucose concentration and can determine the quantity of glucose with lower concentration than the glucose sensor of Comparison Example 2. Although the quantities of glucose dehydrogenase and diaphorase immobilized on the enzyme electrodes are identical, glucose dehydrogenase and diaphorase are fused in the enzyme electrode of Example 2, so that both of the enzymes are held physically near each other. Therefore, it is considered that both of the enzymes can receive and give electrons quickly via NAD/NADH.

### EXAMPLE 3

### Glucose Fuel Cell

Example 3 will be explained with reference to FIG. 10 and FIG. 9. The fuel cell in Example 3 is a glucose fuel cell using glucose as fuel. The anode electrode part of the glucose fuel cell in Example 3 will be explained with reference to FIG. 10. The anode electrode part is constituted as follows.

A conductive base plate 20 is glassy carbon of 0.5 cm². The fusion protein (His-busGDH::ppuDp) prepared in Example 5, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-busGDH::ppuDp (busGDH: 0.3 unit and ppuDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 in Example 3 is made of a platinum plate of 0.5 cm². Electrolyte solution 3 is 50 mM phosphate buffer (pH 7.5) containing 100 mM sodium chloride, 5 mM glucose and 1 mM nicotinamide-adeninedinucleotide. A porous polypropylene film (thickness of 20 μm) is inserted between the anode electrode 15 and the cathode electrode 16, and the electrodes are located in the electrolyte solution 3 (10 mL) in the water jacket cell 1 with the cover 2. The measured temperature is held at 37° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 987 μA/cm²

Maximum power: 100 μW/cm²

### COMPARISON EXAMPLE 3

### Glucose Fuel Cell

Comparison Example 3 will be explained with reference to FIG. 12 and FIG. 9. The fuel cell in Comparison Example 2 is a glucose fuel cell using glucose as fuel. The anode electrode part of the glucose fuel cell in Comparison Example 3 will be explained with reference to FIG. 12. The anode electrode part is constituted as follows.

A conductive base plate 20 is glassy carbon of 0.5 cm². Glucose dehydrogenase (busGDH), diaphorase (ppuDp), and ferrocene-bounded polyallylamine (Fc-PAA) are crosslinked and immobilized on the conductive base plate 20 by PEGDE. Glucose dehydrogenase (busGDH) is glucose dehydrogenase derived from Bacillus subtilis prepared from Comparison Example 1, and diaphorase (ppuDp) is diaphorase derived from Pseudomonas putida. Each of the components is immobilized at following optimal concentrations, respectively. That is to say, the components are prepared at busGDH: 0.3 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The voltage-current characteristic is measured as is similar to Example 3.

The output of the cell is observed as follows.

Short circuit current density: 296 μA/cm²

Maximum power: 31 μW/cm²

From Example 3 and Comparison Example 3, it becomes apparent that the glucose fuel cell of Example 3 has higher current density and can pick up larger current that the glucose fuel cell of Comparison Example 3. Although the quantities of glucose dehydrogenase and diaphorase immobilized on the enzyme electrodes are identical, glucose dehydrogenase and diaphorase are fused in the enzyme electrode of Example 3, so that both of the enzymes are held physically near each other. Therefore, it is considered that both of the enzymes can receive and give electrons quickly via NAD/NADH.

### EXAMPLE 4

### Glucose Electrochemical Reaction Device

Example 4 will be explained with reference to FIG. 10 and FIG. 8. The glucose electrochemical reaction device in Example 4 is a glucose electrochemical reaction device for producing gluconolactone using glucose as a substrate.

The enzyme electrode part of the glucose electrochemical reaction device of Example 4 will be explained with reference to FIG. 10. The enzyme electrode part is constituted as follows. A conductive base plate 20 is glassy carbon of 0.5 cm². The fusion protein (His-busGDH::ppuDp) prepared in Example 1, and ferrocene-bounded polyallylamine (Fc-PAA) are crosslinked and immobilized on the conductive base plate 20 by PEGDE. Each of the components is immobilized at following optimal concentrations, respectively. The components are prepared at His-busGDH::ppuDp (busGDH: 0.3 unit and ppuDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode is used as the reaction electrode 4 of FIG. 8, so as to constitute a glucose electrochemical reaction device. A 3-pole cell using a silver chloride electrode as the reference electrode 6 and a platinum wire as the counter electrode 5 is constituted. 50 mM phosphate buffer (pH 7.5) containing 100 mM sodium chloride, 5 mM glucose and 1 mM nicotinamide-adeninedinucleotide is used as the sample solution 3. The temperature is held at 37° C. by a constant temperature circulation vessel, and the potential of 0.3 VvsAg/AgCl under a nitrogen atmosphere in the water jacket cell 1 is applied for 100 minutes, so that the quantity of the product is determined by high-speed liquid chromatography.

Gluconolactone is detected from the reaction electrolyte. Therefore, it is recognized that a reaction charge volume and a produced gluconolactone volume have high correlation, and the reaction advances quantitatively.

### COMPARISON EXAMPLE 4

### Glucose Electrochemical Reaction Device

Comparison Example 4 will be explained with reference to FIG. 12 and FIG. 8. The glucose electrochemical reaction device in Comparison Example 4 is a glucose electrochemical reaction device for producing gluconolactone using glucose as a substrate. The enzyme electrode part of the glucose electrochemical reaction device of Comparison Example 4 will be explained with reference to FIG. 12.

The enzyme electrode part is constituted as follows. A conductive base plate 20 is glassy carbon of 0.5 cm². Glucose dehydrogenase (busGDH), diaphorase (ppuDp), and ferrocene-bounded polyallylamine (Fc-PAA) are crosslinked and immobilized on the conductive base plate 20 by PEGDE. Glucose dehydrogenase (busGDH) is glucose dehydrogenase derived from Bacillus subtilis prepared in Comparison Example 1, and diaphorase (ppDp) is diaphorase derived from Pseudomonas putida. Each of the components is immobilized at following optimal concentrations, respectively. That is to say, the components are prepared at busGDH: 0.3 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

In this enzyme electrode, as is similar to Example 4, the quantity of the product is determined by high-speed chromatography.

Gluconolactone is detected from the reaction electrolyte. Therefore, it is recognized that a reaction charge volume and a produced gluconolactone volume have high correlation, and the reaction advances quantitatively. However, it is recognized that a reaction charge volume per unit time has a smaller value than that of Example 4. From Example 4 and Comparison Example 4, it becomes apparent that the glucose electrochemical reaction device of Example 4 has a higher reaction charge volume per unit time and can transform glucose dehydrogenase into gluconolactone more efficiently than the glucose electrochemical reaction device of Comparison Example 4. Although the quantities of glucose dehydrogenase and diaphorase immobilized on the enzyme electrodes are identical, glucose dehydrogenase and diaphorase are fused in the enzyme electrode of Example 4, so that both of the enzymes are held physically near each other. Therefore, it is considered that both of the enzymes can receive and give electrons quickly via NAD/NADH.

### EXAMPLE 5

### Preparation of Fusion Protein (His-pfuGDH::phoDp) (SEQ ID NO:22 and 23) of Glucose Dehydrogenase (pfuGDH) Derived from Pyrococcus furiosus and Diaphorase (phoDp) Derived from Pyrococcus horikoshii

Genomic DNA is prepared from Pyrococcus furiosus (ATCC 43587) by a normal method. The genomic DNA is used as a mold and following synthetic oligo DNA is used as a primersynthetic oligo DNAs SEQ ID NO:15 having a sequence recognized by BamHI and SEQ ID NO:16 having a sequence recognized by HindIII are used as primers for performing PCR, so as to obtain a DNA amplified product of 799 bp.

The DNA amplified product is cut by restriction enzymes BamHI and HindIII, and inserted in the same restriction enzyme site of pETDuet-1 (manufactured by Novagen) so as to produce a His-pfuGDH expression vector pETDuet-pfuGDH.

Then, genomic DNA is prepared from Pyrococcus horikoshii KT2440 (ATCC 700860) by a normal method. The genomic DNA is used as a mold and following synthetic oligo DNA is used as a primersynthetic oligo DNAs SEQ ID NO:17 having a sequence recognized by NdeI and SEQ ID NO:18 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pETDuet-pfuGDH so as to produce a His-pfuGDH, phoDp coexpression vector pETDuet-pfuGDH-phoDp.

Then, following 5′ end phosphorylated synthetic oligo DNA is equivalent mixed in a TE buffer, and heated, and then annealed by gradual cooling.

SEQ ID NO:5

SEQ ID NO:6

The DNA fragment is inserted in a recognition site of the restriction enzymes HindIII and NdeI of pETDuet-V pfuGDH-phoDp so as to produce a fusion protein His-pfuGDH::phoDp expression vector pETDuet-pfuGDH::phoDp (SEQ ID NO:19) wherein Hiswherein His-pfuGDH and phoDp are bounded by a spacer sequence SEQ ID NO:145.

Then, the genomic DNA of Pyrococcus furiosus (ATCC 43587) is used as a mold and following synthetic oligo DNA is used as a primersynthetic oligo DNAs SEQ ID NO:20 having a sequence recognized by NcoI and SEQ ID NO:16 having a sequence recognized by HindIII are used as primers for performing PCR, so as to obtain a DNA amplified product of 797 bp.

The DNA amplified product is cut by restriction enzymes NcoI and HindIII, and inserted in the same restriction enzyme site of pCDFDuet-1 (manufactured by Novagen) so as to produce a pfuGDH expression vector pCDFDuet-pfuGDH.

Then, the genomic DNA of Pyrococcus horikoshii KT2440 (ATCC 700860) is used as a mold and following synthetic oligo DNA is used as a primersynthetic oligo DNAs SEQ ID NO:17 having a sequence recognized by NdeI and SEQ ID NO:18 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pCDFDuet-pfuGDH so as to produce a pfuGDH, phoDp coexpression vector pCDFDuet-pfuGDH-phoDp (SEQ ID NO:21).

E. coli BL21 (DE3) is transformed by the expression vectors pETDuet-pfuGDH::phoDp and pCDFDuet-pfuGDH-phoDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Thus obtained fusion protein can be applied to, for example, a glucose sensor, a glucose fuel cell and a glucose electrochemical reaction device.

### COMPARISON EXAMPLE 5

### Preparation of pfuGDH and phoDp as a Control

Genomic DNA is prepared from Pyrococcus furiosus (ATCC 43587) by a normal method. The genomic DNA is used as a mold and following synthetic oligo DNA is used as a primersynthetic oligo DNAs SEQ ID NO:24 having a sequence recognized by NdeI and SEQ ID NO:25 having a sequence recognized by XhoI are used as primers for performing PCR, so as to obtain a DNA amplified product of about 800 bp.

The DNA amplified product is cut by restriction enzymes NdeI and XhoI, and inserted in the same restriction enzyme site of pET-21a(+) (manufactured by Novagen) so as to produce a pfuGDH-His expression vector pET21-pfuGDH.

Then, genomic DNA is prepared from Pyrococcus horikoshii KT2440 (ATCC 700860) by a normal method. The genomic DNA is used as a mold and following synthetic oligo DNA is used as a primersynthetic oligo DNAs SEQ ID NO:17 having a sequence recognized by NdeI and SEQ ID NO:26 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pET-21a(+) (manufactured by Novagen) so as to produce a phoDp-His expression vector pET21-phoDp.

E. coli BL21 (DE3) is transformed according to a normal method by independently using the expression vectors pET21-pfuGDH and pET21-phoDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, the transformant is purified in a manner similar to Example 5.

### EXAMPLE 6

### Glucose Sensor

Example 6 will be explained with reference to FIG. 10 and FIG. 8. The sensor in Example 6 is a glucose sensor for determining the quantity of glucose in a sample solution. The enzyme electrode part of the glucose sensor in Example 6 will be explained with reference to FIG. 10. The enzyme electrode part is constituted as follows.

A conductive base plate 20 is glassy carbon with diameter of 3 mm. The fusion protein (His-pfuGDH::phoDp) prepared in Example 5, and ferrocene-bounded polyallylamine (Fc-PAA) are crosslinked and immobilized on the conductive base plate 20 by PDGDE. Each of the components is immobilized at following optimal concentrations, respectively. That is to say, the components are prepared at His-pfuGDH::phoDp (pfuGDH: 0.3 unit and phoDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is measured as is similar to Example 2. A trend is shown in a solid line C of FIG. 11. That is to say, the line C has a larger inclination than the line A.

### COMPARISON EXAMPLE 6

### Glucose Sensor

Comparison Example 6 will be explained with reference to FIG. 12 and FIG. 8. The sensor in Comparison Example 6 is a glucose sensor for determining the quantity of glucose in a sample solution. The enzyme electrode part of the glucose sensor in Comparison Example 6 will be explained with reference to FIG. 12.

The enzyme electrode part is constituted as follows. A conductive base plate 20 is glassy carbon with diameter of 3 mm. Glucose dehydrogenase (pfuGDH), diaphorase (phoDp) derived from Pyrococcus horikoshii, and ferrocene-bounded polyallylamine (Fc-PAA) are crosslinked and immobilized on the conductive base plate 20 by PEGDE. Glucose dehydrogenase (pfuGDH) is glucose dehydrogenase derived from Pyrococcus furiosus prepared in Comparison Example 5, and diaphorase (phoDp) is diaphorase derived from Pseudomonas putida. Each of the components is immobilized at following optimal concentrations, respectively. That is to say, the components are prepared at pfuGDH: 0.3 unit, phoDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is measured as is similar to Example 6. A trend is shown in a solid line D of FIG. 11. That is to say, the line D has a larger inclination than the line B.

### EXAMPLE 7

### Glucose Fuel Cell

Example 7 will be explained with reference to FIG. 10 and FIG. 9. The fuel cell in Example 7 is a glucose fuel cell using glucose as fuel. The anode electrode part of the glucose fuel cell in Example 7 will be explained with reference to FIG. 10. The anode electrode part is constituted as follows.

A conductive base plate 20 is glassy carbon of 0.5 cm². The fusion protein (His-pfuGDH::phoDp) prepared in Example 5, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-pfuGDH::phoDp (pfuGDH: 0.3 unit and phoDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 in Example 7 is made of a platinum plate of 0.5 cm². Electrolyte solution 3 is 50 mM phosphate buffer (pH 7.5) containing 100 mM sodium chloride, 5 mM glucose and 1 mM nicotinamide-adeninedinucleotide. A porous polypropylene film (thickness of 20 μm) 17 is inserted between the anode electrode 15 and the cathode electrode 16, and the electrodes are located in the electrolyte solution 3 (10 mL) in the water jacket cell 1 with the cover 2. The measured temperature is held at 75° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 1320 μA/cm²

Maximum power: 70125 μW/cm²

### COMPARISON EXAMPLE 7

### Glucose Fuel Cell

Comparison Example 7 will be explained with reference to FIG. 12 and FIG. 9. The fuel cell in Comparison Example 7 is a glucose fuel cell using glucose as fuel. The anode electrode part of the glucose fuel cell in Comparison Example 7 will be explained with reference to FIG. 12. The anode electrode part is constituted as follows.

A conductive base plate 20 is glassy carbon of 0.5 cm². Glucose dehydrogenase (pfuGDH), diaphorase (phoDp) derived from Pyrococcus horikoshii, and ferrocene-bounded polyallylamine (Fc-PAA) are crosslinked and immobilized on the conductive base plate 20 by PEGDE. Glucose dehydrogenase (pfuGDH) is glucose dehydrogenase derived from Pyrococcus furiosus prepared from Comparison Example 5. Each of the components is immobilized at following optimal concentrations, respectively. That is to say, the components are prepared at pfuGDH: 0.3 unit, phoDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The glucose fuel cell of Comparison Example 7 is measured as is similar to Example 7.

The output of the cell is observed as follows.

Short circuit current density: 422 μA/cm²

Maximum power: 40 μW/cm²

From Example 7 and Comparison Example 7, it becomes apparent that the glucose fuel cell of Example 7 has higher current density and can pick up larger current that the glucose fuel cell of Comparison Example 7. Although the quantities of glucose dehydrogenase and diaphorase immobilized on the enzyme electrodes are identical, glucose dehydrogenase and diaphorase are fused in the enzyme electrode of Example 7, so that both of the enzymes are held physically near each other. Therefore, it is considered that both of the enzymes can receive and give electrons quickly via NAD/NADH.

### EXAMPLE 8

### Glucose Electrochemical Reaction Device

Example 8 will be explained with reference to FIG. 10 and FIG. 8. The glucose electrochemical reaction device in Example 8 is a glucose electrochemical reaction device for producing gluconolactone using glucose as a substrate. The enzyme electrode part of the glucose electrochemical reaction device of Example 8 will be explained with reference to FIG. 10. The enzyme electrode part is constituted as follows.

A conductive base plate 20 is glassy carbon of 0.5 cm². The fusion protein (His-pfuGDH::phoDp) prepared in Example 5, and ferrocene-bounded polyallylamine (Fc-PAA) are crosslinked and immobilized on the conductive base plate 20 by PEGDE. Each of the components is immobilized at following optimal concentrations, respectively. The components are prepared at His-pfuGDH::phoDp (pfuGDH: 0.3 unit and phoDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

As is similar to Example 4, the quantity of the product is determined by high-speed chromatography using this enzyme electrode. Gluconolactone is detected from the reaction electrolyte. Therefore, it is recognized that a reaction charge volume and a produced gluconolactone volume have high correlation, and the reaction advances quantitatively.

### COMPARISON EXAMPLE 8

### Glucose Electrochemical Reaction Device

Comparison Example 8 will be explained with reference to FIG. 12 and FIG. 8. The glucose electrochemical reaction device in Comparison Example 8 is a glucose electrochemical reaction device for producing gluconolactone using glucose as a substrate. The enzyme electrode part of the glucose electrochemical reaction device of Comparison Example 8 will be explained with reference to FIG. 12. The enzyme electrode part is constituted as follows.

A conductive base plate 20 is glassy carbon of 0.5 cm². Glucose dehydrogenase (pfuGDH), diaphorase (phoDp) derived from Pyrococcus horikoshii, and ferrocene-bounded polyallylamine (Fc-PAA) are crosslinked and immobilized on the conductive base plate 20 by PEGDE. Glucose dehydrogenase (pfuGDH) is glucose dehydrogenase derived from Pyrococcus furiosus prepared in Comparison Example 5. Each of the components is immobilized at following optimal concentrations, respectively. That is to say, the components are prepared at pfuGDH: 0.3 unit, phoDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

Using this enzyme electrode, as is similar to Example 8, the quantity of the product is determined by high-speed chromatography.

Gluconolactone is detected from the reaction electrolyte. Therefore, it is recognized that a reaction charge volume and a produced gluconolactone volume have high correlation, and the reaction advances quantitatively. However, it is recognized that a reaction charge volume per unit time has a smaller value than that of Example 8.

From Example 8 and Comparison Example 8, it becomes apparent that the glucose electrochemical reaction device of Example 8 has a higher reaction charge volume per unit time and can transform glucose dehydrogenase into gluconolactone more efficiently than the glucose electrochemical reaction device of Comparison Example 8. Although the quantities of glucose dehydrogenase and diaphorase immobilized on the enzyme electrodes are identical, glucose dehydrogenase and diaphorase are fused in the enzyme electrode of Example 8, so that both of the enzymes are held physically near each other. Therefore, it is considered that both of the enzymes can receive and give electrons quickly via NAD/NADH.

### EXAMPLE 9

### Preparation of Fusion Protein (His-sceADH::ppuDp (SEQ ID NO:34 and 35) of Alcohol Dehydrogenase (ppuADH) Derived from Saccharomyces cerevisiae and Diaphorase (ppuDp) Derived from Pseudomonas putida

Genomic DNA is prepared from Saccharomyces cerevisiae (ATCC 47058) by a normal method. The genomic DNA is used as a mold and following synthetic oligo DNA is used as a primersynthetic oligo DNAs SEQ ID NO:27 having a sequence recognized by BamHI and SEQ ID NO:28 having a sequence recognized by HindIII are used as primers for performing PCR, so as to obtain a DNA amplified product of 1075 bp.

The DNA amplified product is cut by restriction enzymes BamHI and HindIII, and inserted in the same restriction enzyme site of pETDuet-1 (manufactured by Novagen) so as to produce a His-sceADH expression vector pETDuet-sceADH.

Then, genomic DNA is prepared from Pseudomonas putida KT2440 (ATCC 47054) by a normal method. The genomic DNA is used as a mold and following synthetic oligo DNA is used as a primersynthetic oligo DNAs SEQ ID NO:3 having a sequence recognized by NdeI and SEQ ID NO:4 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pETDuet-sceADH so as to produce a His-sceADH, ppuDp coexpression vector pETDuet-sceADH-ppuDp.

Then, following 5′ end phosphorylated synthetic oligo DNA is equivalent mixed in a TE buffer, and heated, and then annealed by gradual cooling.

SEQ ID NO:29

SEQ ID NO:30

The DNA fragment is inserted in a recognition site of the restriction enzymes HindIII and NdeI of pETDuet-sceADH-ppuDp so as to produce a fusion protein His-sceADH::ppuDp expression vector pETDuet-sceADH::ppuDp (SEQ ID NO:31) wherein Hiswherein His-sceADH and ppuDp are bounded by a spacer sequence SEQ ID NO:146.

Then, the genomic DNA of Saccharomyces cerevisiae (ATCC 47058) is used as a mold and following synthetic oligo DNA is used as a primersynthetic oligo DNAs SEQ ID NO:32 having a sequence recognized by NcoI and SEQ ID NO:28 having a sequence recognized by HindIII are used as primers for performing PCR, so as to obtain a DNA amplified product of 1073 bp.

The DNA amplified product is cut by restriction enzymes NcoI and HindIII, and inserted in the same restriction enzyme site of pCDFDuet-1 (manufactured by Novagen) so as to produce a sceADH expression vector pCDFDuet-sceADH.

Then, the genomic DNA of Pseudomonas putida KT2440 (ATCC 47054) is used as a mold and following synthetic oligo DNA is used as a primersynthetic oligo DNAs SEQ ID NO:3 having a sequence recognized by NdeI and SEQ ID NO:4 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pCDFDuet-sceADH so as to produce a sceADH, ppuDp coexpression vector pCDFDuet-sceADH-ppuDp (SEQ ID NO:33).

E. coli BL21 (DE3) is transformed by the expression vectors pETDuet-sceADH::ppuDp and pCDFDuet-sceADH-ppuDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and shake-cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### COMPARISON EXAMPLE 9

### Preparation of sceADH and ppuDp as a Control

Genomic DNA is prepared from Saccharomyces cerevisiae (ATCC 47058) by a normal method. The genomic DNA is used as a mold and following synthetic oligo DNA is used as a primersynthetic oligo DNAs SEQ ID NO:36 having a sequence recognized by NdeI and SEQ ID NO:37 having a sequence recognized by XhoI are used as primers for performing PCR, so as to obtain a DNA amplified product of about 1074 bp.

The DNA amplified product is cut by restriction enzymes NdeI and XhoI, and inserted in the same restriction enzyme site of pET-21a(+) (manufactured by Novagen) so as to produce a sceADH-His expression vector pET21-sceADH.

Then, genomic DNA is prepared from Pseudomonas putida KT2440 (ATCC 47054) by a normal method. The genomic DNA is used as a mold and following synthetic oligo DNA is used as a primersynthetic oligo DNAs SEQ ID NO:3 having a sequence recognized by NdeI and SEQ ID NO:14 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pET-21a(+) (manufactured by Novagen) so as to produce a ppuDp-His expression vector pET21-ppuDp.

E. coli BL21 (DE3) is transformed according to a normal method by independently using the expression vectors pET21-sceADH and pET21-ppuDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### EXAMPLE 10

### Alcohol Sensor

Example 10 will be explained with reference to FIG. 16 and FIG. 8. The sensor in Example 10 is an alcohol sensor for determining the quantity of alcohol in a sample solution. The enzyme electrode part of the alcohol sensor in Example 10 will be explained with reference to FIG. 16. The enzyme electrode part is constituted as follows.

A conductive base plate 20 is glassy carbon with diameter of 3 mm. The fusion protein (His-sceADH::ppuDp) prepared in Example 9, and ferrocene-bounded polyallylamine (Fc-PAA) are crosslinked and immobilized on the conductive base plate 20 by PDGDE. Each of the components is immobilized at following optimal concentrations, respectively. That is to say, the components are prepared at His-sceADH::ppuDp (sceADH: 0.3 unit and ppuDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute an alcohol sensor. The counter electrode 5 is formed of a platinum wire, and the reference electrode 6 is made of a silver/silver chloride electrode. These electrodes measure current and the like via a potentiostat 10, respectively. 0.1 M PIPES-NaOH buffer aqueous solution (pH 7.5) containing a predetermined concentration of alcohol (ethanol) and 1 mM NAD is used for the sample solution 3. The measured temperature is held at 37° C. by a constant temperature circulation vessel.

The quantity is determined using the above-mentioned alcohol sensor as follows. A potential of 300 mV is applied to the reference electrode 6 in the reaction electrode 4. At that time, alcohol in the sample solution 3 is oxidized to acetaldehyde in the presence of alcohol dehydrogenase, and NAD is reduced to NADH by this reaction. Then, NADH is oxidized to NAD in the presence of diaphorase fused to the alcohol dehydrogenase. Ferrocene as an electron transfer mediator is oxidized by this reaction so as to produce a ferricinium ions. The ferricinium ions receive electrons from the reaction electrode 4 and are reduced to ferrocene (because the potential of 300 mV is applied to the reference electrode 6 in the reaction electrode 4). The current by movement of the electrons at the reaction electrode 4 is measured, so as to measure the concentration of alcohol in the sample solution.

### COMPARISON EXAMPLE 10

### Alcohol Sensor

Comparison Example 10 will be explained with reference to FIG. 18 and FIG. 8. The sensor in Comparison Example 10 is an alcohol sensor for determining the quantity of alcohol in a sample solution. The enzyme electrode part of the alcohol sensor in Comparison Example 10 will be explained with reference to FIG. 18. The enzyme electrode part is constituted as follows.

A conductive base plate 20 is glassy carbon with diameter of 3 mm. Alcohol dehydrogenase (sceADH), diaphorase (ppuDp) derived from Pseudomonas putida, and ferrocene-bounded polyallylamine (Fc-PAA) are crosslinked and immobilized on the conductive base plate 20 by PEGDE. Alcohol dehydrogenase (sceADH) is alcohol dehydrogenase derived from Pyrococcus furiosus prepared in Comparison Example 9. Each of the components is immobilized at following optimal concentrations, respectively. That is to say, the components are prepared at sceADH: 0.3 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

When the enzyme electrode part is used to constitute an alcohol sensor and measurement is done as is similar to Example 10, the difference between the sensitivities of the sensor of Example 10 and the sensor of Comparison Example 10 is similar to the relationship between the solid lines A and B of FIG. 11.

### EXAMPLE 11

### Alcohol Fuel Cell

Example 11 will be explained with reference to FIG. 16 and FIG. 9. The fuel cell in Example 11 is an alcohol fuel cell using alcohol as fuel. The anode electrode part of the alcohol fuel cell in Example 11 will be explained with reference to FIG. 16. The anode electrode part is constituted as follows.

A conductive base plate 20 is glassy carbon of 0.5 cm². The fusion protein (His-sceADH::ppuDp) prepared in Example 9, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-sceADH::ppuDp (sceADH: 0.3 unit and ppuDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 of the alcohol fuel cell in Example 11 is made of a platinum plate of 0.5 cm². Electrolyte solution 3 is 50 mM phosphate buffer (pH 7.5) containing 100 mM sodium chloride, 5 mM alcohol (ethanol) and 1 mM nicotinamide-adeninedinucleotide. A porous polypropylene film (thickness of 20 μm) 17 is inserted between the anode electrode 15 and the cathode electrode 16, and the electrodes are located in the electrolyte solution 3 (10 mL) in the water jacket cell 1 with the cover 2. The measured temperature is held at 75° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 820 μA/cm²

Maximum power: 79 μW/cm²

### COMPARISON EXAMPLE 11

### Alcohol Fuel Cell

Comparison Example 11 will be explained with reference to FIG. 18 and FIG. 9. The fuel cell in Comparison Example 11 is an alcohol fuel cell using alcohol as fuel. The anode electrode part of the alcohol fuel cell in Comparison Example 11 will be explained with reference to FIG. 18. The anode electrode part is constituted as follows.

A conductive base plate 20 is glassy carbon of 0.5 cm². Alcohol dehydrogenase (sceADH), diaphorase (ppuDp) derived from Pseudomonas putida, and ferrocene-bounded polyallylamine (Fc-PAA) are crosslinked and immobilized on the conductive base plate 20 by PEGDE. Alcohol dehydrogenase (sceADH) is alcohol dehydrogenase derived from Saccharomyces cerevisiae prepared from Comparison Example 9. Each of the components is immobilized at following optimal concentrations, respectively. That is to say, the components are prepared at sceADH: 0.3 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them. As is similar to Example 11, the voltage-current characteristic is measured.

The output of the cell is observed as follows.

Short circuit current density: 245 μA/cm²

Maximum power: 24 μW/cm²

From Example 11 and Comparison Example 11, it becomes apparent that the alcohol fuel cell of Example 11 has higher current density and can pick up larger current that the alcohol fuel cell of Comparison Example 11. Although the quantities of alcohol dehydrogenase and diaphorase immobilized on the enzyme electrodes are identical, alcohol dehydrogenase and diaphorase are fused in the enzyme electrode of Example 11, so that both of the enzymes are held physically near each other. Therefore, it is considered that both of the enzymes can receive and give electrons quickly via NAD/NADH.

### EXAMPLE 12

### Alcohol Electrochemical Reaction Device

Example 12 will be explained with reference to FIG. 16 and FIG. 8. The alcohol electrochemical reaction device in Example 12 is an alcohol electrochemical reaction device for producing acetaldehyde using alcohol (ethanol) as a substrate. The enzyme electrode part of the alcohol electrochemical reaction device of Example 12 will be explained with reference to FIG. 16. The enzyme electrode part is constituted as follows.

A conductive base plate 20 is glassy carbon of 0.5 cm². The fusion protein (His-sceADH::ppuDp) prepared in Example 9, and ferrocene-bounded polyallylamine (Fc-PAA) are crosslinked and immobilized on the conductive base plate 20 by PEGDE. Each of the components is immobilized at following optimal concentrations, respectively. The components are prepared at His-sceADH::ppuDp (sceADH: 0.3 unit and ppuDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode is used as the reaction electrode 4 of FIG. 8, so as to constitute an alcohol electrochemical reaction device. A 3-pole cell using a silver chloride electrode as the reference electrode 6 and a platinum wire as the counter electrode 5 is constituted. 50 mM phosphate buffer (pH 7.5) containing 100 mM sodium chloride, 5 mM alcohol and 1 mM nicotinamide-adeninedinucleotide is used as the sample solution 3. The temperature is held at 37° C. by a constant temperature circulation vessel, and the potential of 0.3 VvsAg/AgCl under a nitrogen atmosphere in the water jacket cell 1 is applied for 100 minutes, so that the quantity of the product is determined by high-speed liquid chromatography. Acetaldehyde is detected from the reaction electrolyte. Therefore, it is recognized that a reaction charge volume and a produced acetaldehyde volume have high correlation, and the reaction advances quantitatively.

### COMPARISON EXAMPLE 12

### Alcohol Electrochemical Reaction Device

Comparison Example 12 will be explained with reference to FIG. 18 and FIG. 8. The alcohol electrochemical reaction device in Comparison Example 12 is an alcohol electrochemical reaction device for producing acetaldehyde using alcohol (ethanol) as a substrate. The enzyme electrode part of the alcohol electrochemical reaction device of Comparison Example 12 will be explained with reference to FIG. 12. The enzyme electrode part is constituted as follows.

A conductive base plate 20 is glassy carbon of 0.5 cm². Alcohol dehydrogenase (sceADH), diaphorase (ppuDp) derived from Pseudomonas putida, and ferrocene-bounded polyallylamine (Fc-PAA) are crosslinked and immobilized on the conductive base plate 20 by PEGDE. Alcohol dehydrogenase (sceADH) is alcohol dehydrogenase derived from Saccharomyces cerevisiae prepared in Comparison Example 9. Each of the components is immobilized at following optimal concentrations, respectively. That is to say, the components are prepared at sceADH: 0.3 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

Using this enzyme electrode, as is similar to Example 12, the quantity of the product is determined by high-speed chromatography.

Acetaldehyde is detected from the reaction electrolyte. Therefore, it is recognized that a reaction charge volume and a produced acetaldehyde volume have high correlation, and the reaction advances quantitatively. However, it is recognized that a reaction charge volume per unit time has a smaller value than that of Example 12.

From Example 12 and Comparison Example 12, it becomes apparent that the alcohol electrochemical reaction device of Example 12 has a higher reaction charge volume per unit time and can transform alcohol dehydrogenase into acetaldehyde more efficiently than the alcohol electrochemical reaction device of Comparison Example 12. Although the quantities of alcohol dehydrogenase and diaphorase immobilized on the enzyme electrodes are identical, alcohol dehydrogenase and diaphorase are fused in the enzyme electrode of Example 12, so that both of the enzymes are held physically near each other. Therefore, it is considered that both of the enzymes can receive and give electrons quickly via NAD/NADH.

### EXAMPLE 13

### Preparation of Fusion Protein (His-pfuADH::phoDp) (SEQ ID NO:43 and 44) of Alcohol Dehydrogenase (pfuADH) Derived from Pyrococcus furiosus and Diaphorase (phoDp) Derived from Pyrococcus horikoshii

Genomic DNA is prepared from Pyrococcus furiosus (ATCC 43587) by a normal method. The genomic DNA is used as a mold and following synthetic oligo DNA is used as a primersynthetic oligo DNAs SEQ ID NO:38 having a sequence recognized by BamHI and SEQ ID NO:39 having a sequence recognized by HindIII are used as primers for performing PCR, so as to obtain a DNA amplified product of 1147 bp.

The DNA amplified product is cut by restriction enzymes BamHI and HindIII, and inserted in the same restriction enzyme site of pETDuet-1 (manufactured by Novagen) so as to produce a His-pfuADH expression vector pETDuet-pfuADH.

Then, genomic DNA is prepared from Pyrococcus horikoshii KT2440 (ATCC 700860) by a normal method. The genomic DNA is used as a mold and following synthetic oligo DNA is used as a primersynthetic oligo DNAs SEQ ID NO:17 having a sequence recognized by NdeI and SEQ ID NO:18 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pETDuet-pfuADH so as to produce a His-pfuADH, phoDp coexpression vector pETDuet-pfuADH-phoDp.

Then, following 5′ end phosphorylated synthetic oligo DNA is equivalent mixed in a TE buffer, and heated, and then annealed by gradual cooling.

SEQ ID NO:29

SEQ ID NO:30

The DNA fragment is inserted in a recognition site of the restriction enzymes HindIII and NdeI of pETDuet-pfuADH-phoDp so as to produce a fusion protein His-pfuADH::phoDp expression vector pETDuet-pfuADH::phoDp (SEQ ID NO:40) wherein Hiswherein His-pfuADH and phoDp are bounded by a spacer sequence SEQ ID NO:146.

Then, the genomic DNA of Pyrococcus furiosus (ATCC 43587) is used as a mold and following synthetic oligo DNA is used as a primersynthetic oligo DNAs SEQ ID NO:41 having a sequence recognized by NcoI and SEQ ID NO:39 having a sequence recognized by HindIII are used as primers for performing PCR, so as to obtain a DNA amplified product of 1145 bp.

The DNA amplified product is cut by restriction enzymes NcoI and HindIII, and inserted in the same restriction enzyme site of pCDFDuet-1 (manufactured by Novagen) so as to produce a pfuADH expression vector pCDFDuet-pfuADH.

Then, the genomic DNA of Pyrococcus horikoshii KT2440 (ATCC 700860) is used as a mold and following synthetic oligo DNA is used as a primersynthetic oligo DNAs SEQ ID NO:17 having a sequence recognized by NdeI and SEQ ID NO:18 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pCDFDuet-pfuADH so as to produce a pfuADH, phoDp coexpression vector pCDFDuet-pfuADH-phoDp (SEQ ID NO:42).

E. coli BL21 (DE3) is transformed by the expression vectors pETDuet-pfuADH::phoDp and pCDFDuet-pfuADH-phoDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

By using thus obtained fusion protein derived from thermophile, an alcohol sensor, a fuel cell and the like can be manufactured in a manner similar to the above-mentioned examples.

### EXAMPLE 17

### Preparation of Fusion Protein (His-lplLDH::goxDp) (SEQ ID NO:56 and 57) of Lactic Dehydrogenase (lplLDH) Derived from Lactobacillus plantarum and Diaphorase (goxDp) Derived from Gluconobacter oxydans

Genomic DNA is prepared from Lactobacillus plantarum (ATCC 10241) by a normal method. The genomic DNA is used as a mold and following synthetic oligo DNA is used as a primersynthetic oligo DNAs SEQ ID NO:47 having a sequence recognized by BamHI and SEQ ID NO:48 having a sequence recognized by HindIII are used as primers for performing PCR, so as to obtain a DNA amplified product of 982 bp.

The DNA amplified product is cut by restriction enzymes BamHI and HindIII, and inserted in the same restriction enzyme site of pETDuet-1 (manufactured by Novagen) so as to produce a His-lplLDH expression vector pETDuet-lplLDH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:49 having a sequence recognized by NdeI and SEQ ID NO:50 having a sequence recognized by XhoI are used as primers for performing PCR, so as to obtain a DNA amplified product of 1428 bp.

The DNA amplified product is cut by restriction enzymes NdeI and XhoI, and inserted in the same restriction enzyme site of pETDuet-lplLDH so as to produce a His-lplLDH, goxbp coexpression vector pETDuet-lplLDH-goxDp.

Then, following 5′ end phosphorylated synthetic oligo DNA is equivalent mixed in a TE buffer, and heated, and then annealed by gradual cooling.

SEQ ID NO:51

SEQ ID NO:52

The DNA fragment is inserted in a recognition site of the restriction enzymes HindIII and NdeI of pETDuet-lplLDH-goxDp so as to produce a fusion protein His-lplLDH::goxDp expression vector pETDuet-lplLDH::goxDp (SEQ ID NO:53) wherein Hiswherein His-lplLDH and goxDp are bounded by a spacer sequence SEQ ID NO:142.

Then, the genomic DNA of Lactobacillus plantarum (ATCC 10241) is used as a mold and following synthetic oligo DNA is used as a primersynthetic oligo DNAs SEQ ID NO:54 having a sequence recognized by NcoI and SEQ ID NO:48 having a sequence recognized by HindIII are used as primers for performing PCR, so as to obtain a DNA amplified product of 980 bp.

The DNA amplified product is cut by restriction enzymes NcoI and HindIII, and inserted in the same restriction enzyme site of pCDFDuet-1 (manufactured by Novagen) so as to produce a lplLDH expression vector pCDFDuet-lplLDH.

Then, the genomic DNA of Gluconobacter oxydans (ATCC 621H) is used as a mold and following synthetic oligo DNA is used as a primersynthetic oligo DNAs SEQ ID NO:49 having a sequence recognized by NdeI and SEQ ID NO:50 having a sequence recognized by XhoI are used as primers for performing PCR, so as to obtain a DNA amplified product of 1248 bp.

The DNA amplified product is cut by restriction enzymes NdeI and XhoI, and inserted in the same restriction enzyme site of pCDFDuet-lplLDH so as to produce a lplLDH, goxDp coexpression vector pCDFDuet-lplLDH-goxDp (SEQ ID NO:55).

E. coli BL21 (DE3) is transformed by the expression vectors pETDuet-lplLDH::goxDp and pCDFDuet-lplLDH-goxDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### COMPARISON EXAMPLE 17

### Preparation of lplLDH and goxDp as a Control

Genomic DNA is prepared from Lactobacillus plantarum (ATCC 10241) by a normal method. The genomic DNA is used as a mold and following synthetic oligo DNA is used as a primersynthetic oligo DNAs SEQ ID NO:58 having a sequence recognized by NdeI and SEQ ID NO:59 having a sequence recognized by XhoI are used as primers for performing PCR, so as to obtain a DNA amplified product of about 980 bp.

The DNA amplified product is cut by restriction enzymes NdeI and XhoI, and inserted in the same restriction enzyme site of pET-21a(+) (manufactured by Novagen) so as to produce a lplLDH-His expression vector pET21-lplLDH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:49 having a sequence recognized by NdeI and SEQ ID NO:60 having a sequence recognized by XhoI are used as primers for performing PCR, so as to obtain a DNA amplified product of about 1420 bp.

The DNA amplified product is cut by restriction enzymes NdeI and XhoI, and inserted in the same restriction enzyme site of pET-21a(+) (manufactured by Novagen) so as to produce a goxDp-His expression vector pET21-goxDp.

E. coli BL21 (DE3) is transformed according to a normal method by independently using the expression vectors pET21-lplLDH and pET21-goxDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### EXAMPLE 18

### Lactic Acid Sensor

Example 18 will be explained with reference to FIG. 22 and FIG. 8. The sensor in Example 18 is a lactic acid sensor for determining the quantity of lactic acid in a sample solution. The enzyme electrode part of the lactic acid sensor in Example 18 will be explained with reference to FIG. 22.

The enzyme electrode part is constituted as follows. A conductive base plate 20 is glassy carbon with diameter of 3 mm. The fusion protein (His-lplLDH::goxDp) prepared in Example 17, and ferrocene-bounded polyallylamine (Fc-PAA) are crosslinked and immobilized on the conductive base plate 20 by PDGDE. Each of the components is immobilized at following optimal concentrations, respectively. That is to say, the components are prepared at His-lplLDH::goxDp (lplLDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute a lactic acid sensor. The counter electrode 5 is formed of a platinum wire, and the reference electrode 6 is made of a silver/silver chloride electrode. These electrodes measure current and the like via a potentiostat 10, respectively. 0.1 M PIPES-NaOH buffer aqueous solution (pH 7.5) containing a predetermined concentration of lactic acid (L-lactic acid) and 1 mM NAD is used for the sample solution 3. The measured temperature is held at 30° C. by a constant temperature circulation vessel.

The quantity is determined using the above-mentioned lactic acid sensor as follows. A potential of 300 mV is applied to the reference electrode 6 in the reaction electrode 4. At that time, lactic acid in the sample solution 3 is oxidized to pyruvic acid in the presence of lactic dehydrogenase, and NAD is reduced to NADH by this reaction. Then, NADH is oxidized to NAD in the presence of diaphorase fused to the lactic dehydrogenase. Ferrocene as an electron transfer mediator is oxidized by this reaction so as to produce a ferricinium ions. The ferricinium ions receive electrons from the reaction electrode 4 and are reduced to ferrocene (because the potential of 300 mV is applied to the reference electrode 6 in the reaction electrode 4). The current by movement of the electrons at the reaction electrode 4 is measured, so as to measure the concentration of lactic acid in the sample solution.

### COMPARISON EXAMPLE 18

### Lactic Acid Sensor

Comparison Example 18 will be explained with reference to FIG. 22 and FIG. 8. The sensor in Comparison Example 18 is a lactic acid sensor for determining the quantity of lactic acid in a sample solution. The enzyme electrode part of the lactic acid sensor in Comparison Example 18 will be explained with reference to FIG. 22.

The enzyme electrode part is constituted as follows. A conductive base plate 20 is glassy carbon with diameter of 3 mm. Lactic dehydrogenase (lplLDH), diaphorase (goxDp), and ferrocene-bounded polyallylamine (Fc-PAA) are crosslinked and immobilized on the conductive base plate 20 by PEGDE. Lactic dehydrogenase (lplLDH) is lactic dehydrogenase derived from Lactobacillus plantarum prepared in Comparison Example 17, and diaphorase (goxDp) is diaphorase derived from Gluconobacter oxydans. Each of the components is immobilized at following optimal concentrations, respectively. That is to say, the components are prepared at lplLDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

In this enzyme electrode part, as is similar to Example 18, the concentration of lactic acid in the sample solution is measured. The difference between the results of Example 18 and Comparison Example 18 is similar to the difference between the lines A and B of FIG. 11.

From Example 18 and Comparison Example 18, it becomes apparent that the lactic acid sensor of Example 18 has higher sensitivity to the lactic acid concentration and can determine the quantity of lactic acid with lower concentration than the lactic acid sensor of Comparison Example 18. Although the quantities of lactic dehydrogenase and diaphorase immobilized on the enzyme electrodes are identical, lactic dehydrogenase and diaphorase are fused in the enzyme electrode of Example 18, so that both of the enzymes are held physically near each other. Therefore, it is considered that both of the enzymes can receive and give electrons quickly via NAD/NADH.

### EXAMPLE 19

### Lactic Acid Fuel Cell

Example 19 will be explained with reference to FIG. 22 and FIG. 9. The fuel cell in Example 19 is a lactic acid fuel cell using lactic acid as fuel. The anode electrode part of the lactic acid fuel cell in Example 19 will be explained with reference to FIG. 22.

The anode electrode part is constituted as follows. A conductive base plate 20 is glassy carbon of 0.5 cm². The fusion protein (His-lplLDH::goxDp) prepared in Example 17, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-lplLDH::goxDp (lplLDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 of the lactic acid fuel cell in Example 19 is made of a platinum plate of 0.5 cm². Electrolyte solution 3 is 50 mM phosphate buffer (pH 7.5) containing 100 mM sodium chloride, 5 mM lactic acid (L-lactic acid) and 1 mM nicotinamide-adeninedinucleotide. A porous polypropylene film (thickness of 20 μm) 17 is inserted between the anode electrode 15 and the cathode electrode 16, and the electrodes are located in the electrolyte solution 3 (10 mL) in the water jacket cell 1 with the cover 2. The measured temperature is held at 30° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 1330 μA/cm²

Maximum power: 120 μW/cm²

### COMPARISON EXAMPLE 19

### Lactic Acid Fuel Cell

Comparison Example 19 will be explained with reference to FIG. 24 and FIG. 9. The fuel cell in Comparison Example 19 is a lactic acid fuel cell using lactic acid as fuel. The anode electrode part of the lactic acid fuel cell in Comparison Example 19 will be explained with reference to FIG. 24.

The anode electrode part is constituted as follows. A conductive base plate 20 is glassy carbon of 0.5 cm². Lactic dehydrogenase (lplLDH), diaphorase (goxDp), and ferrocene-bounded polyallylamine (Fc-PAA) are crosslinked and immobilized on the conductive base plate 20 by PEGDE. Lactic dehydrogenase (lplLDH) is lactic dehydrogenase derived from Lactobacillus plantarum prepared in Comparison Example 17. Diaphorase (goxDp) is diaphorase derived from Gluconobacter oxydans. Each of the components is immobilized at following optimal concentrations, respectively. That is to say, the components are prepared at lplLDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

As is similar to Example 19, the voltage-current characteristic is measured using the cathode electrode of the lactic acid fuel cell of Comparison Example 19.

The output of the cell is observed as follows.

Short circuit current density: 396 μA/cm²

Maximum power: 37 μW/cm²

From Example 19 and Comparison Example 19, it becomes apparent that the lactic acid fuel cell of Example 19 has higher current density and can pick up larger current that the lactic acid fuel cell of Comparison Example 19. Although the quantities of lactic dehydrogenase and diaphorase immobilized on the enzyme electrodes are identical, lactic dehydrogenase and diaphorase are fused in the enzyme electrode of Example 19, so that both of the enzymes are held physically near each other. Therefore, it is considered that both of the enzymes can receive and give electrons quickly via NAD/NADH.

### EXAMPLE 20

### Lactic Acid Electrochemical Reaction Device

Example 20 will be explained with reference to FIG. 22 and FIG. 8. The lactic acid electrochemical reaction device in Example 20 is a lactic acid electrochemical reaction device for producing pyruvic acid using lactic acid (L-lactic acid) as a substrate. The enzyme electrode part of the lactic acid electrochemical reaction device of Example 20 will be explained with reference to FIG. 22. The enzyme electrode part is constituted as follows.

A conductive base plate 20 is glassy carbon of 0.5 cm² The fusion protein (His-lplLDH::goxDp) prepared in Example 17, and ferrocene-bounded polyallylamine (Fc-PAA) are crosslinked and immobilized on the conductive base plate 20 by PEGDE. Each of the components is immobilized at following optimal concentrations, respectively. The components are prepared at His-lplLDH::goxDp (lplLDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode is used as the reaction electrode 4 of FIG. 8, so as to constitute a lactic acid electrochemical reaction device. A 3-pole cell using a silver chloride electrode as the reference electrode 6 and a platinum wire as the counter electrode 5 is constituted. 50 mM phosphate buffer (pH 7.5) containing 100 mM sodium chloride, 5 mM lactic acid and 1 mM nicotinamide-adeninedinucleotide is used as the sample solution 3. The temperature is held at 30° C. by a constant temperature circulation vessel, and the potential of 0.3 VvsAg/AgCl under a nitrogen atmosphere in the water jacket cell 1 is applied for 100 minutes, so that the quantity of the product is determined by high-speed liquid chromatography.

Pyruvic acid is detected from the reaction electrolyte. Therefore, it is recognized that a reaction charge volume and a produced pyruvic acid volume have high correlation, and the reaction advances quantitatively.

### COMPARISON EXAMPLE 20

### Lactic Acid Electrochemical Reaction Device

Comparison Example 20 will be explained with reference to FIG. 24 and FIG. 8. The lactic acid electrochemical reaction device in Comparison Example 20 is a lactic acid electrochemical reaction device for producing pyruvic acid using lactic acid (L-latic acid) as a substrate. The enzyme electrode part of the lactic acid electrochemical reaction device of Comparison Example 20 will be explained with reference to FIG. 24. The enzyme electrode part is constituted as follows.

A conductive base plate 20 is glassy carbon of 0.5 cm². Lactic dehydrogenase (lplLDH), diaphorase (goxDp), and ferrocene-bounded polyallylamine (Fc-PAA) are crosslinked and immobilized on the conductive base plate 20 by PEGDE. Lactic dehydrogenase (lplLDH) is lactic dehydrogenase derived from Lactobacillus plantarum prepared in Comparison Example 17, and diaphorase (goxDp) is a diaphorase derived from Gluconobacter oxydans. Each of the components is immobilized at following optimal concentrations, respectively. That is to say, the components are prepared at lplLDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

Using this enzyme electrode, as is similar to Example 20, the quantity of the product is determined by high-speed chromatography.

Pyruvic acid is detected from the reaction electrolyte. Therefore, it is recognized that a reaction charge volume and a produced pyruvic acid volume have high correlation, and the reaction advances quantitatively. However, it is recognized that a reaction charge volume per unit time has a smaller value than that of Example 20.

From Example 20 and Comparison Example 20, it becomes apparent that the lactic acid electrochemical reaction device of Example 20 has a higher reaction charge volume per unit time and can transform lactic dehydrogenase into pyruvic acid more efficiently than the lactic acid electrochemical reaction device of Comparison Example 20. Although the quantities of lactic dehydrogenase and diaphorase immobilized on the enzyme electrodes are identical, lactic dehydrogenase and diaphorase are fused in the enzyme electrode of Example 20, so that both of the enzymes are held physically near each other. Therefore, it is considered that both of the enzymes can receive and give electrons quickly via NAD/NADH.

### EXAMPLE 21

### Preparation of Fusion Protein (His-tmaLDH::tmaDp) (SEQ ID NO:68 and 60) of Lactic Dehydrogenase (tmaLDH) Derived from Themotoga maritima and Diaphorase (tmaDp) Derived from Themotoga maritima

Genomic DNA is prepared from Themotoga maritima (ATCC 43589) by a normal method. The genomic DNA is used as a mold and following synthetic oligo DNA is used as a primersynthetic oligo DNAs SEQ ID NO:61 having a sequence recognized by BamHI and SEQ ID NO:62 having a sequence recognized by HindIII are used as primers for performing PCR, so as to obtain a DNA amplified product of 979 bp.

The DNA amplified product is cut by restriction enzymes BamHI and HindIII, and inserted in the same restriction enzyme site of pETDuet-1 (manufactured by Novagen) so as to produce a His-tmaLDH expression vector pETDuet-tmaLDH.

Then, genomic DNA is prepared from Themotoga maritima (ATCC 43589) by a normal method. The genomic DNA is used as a mold and following synthetic oligo DNA is used as a primersynthetic oligo DNAs SEQ ID NO:63 having a sequence recognized by NdeI and SEQ ID NO:64 having a sequence recognized by XhoI are used as primers for performing PCR, so as to obtain a DNA amplified product of 1371 bp.

The DNA amplified product is cut by restriction enzymes NdeI and XhoI, and inserted in the same restriction enzyme site of pETDuet-tmaLDH so as to produce a His-tmaLDH, tmaDp coexpression vector pETDuet-tmaLDH-tmaDp.

Then, following 5′ end phosphorylated synthetic oligo DNA is equivalent mixed in a TE buffer, and heated, and then annealed by gradual cooling.

SEQ ID NO:51

SEQ ID NO:52

The DNA fragment is inserted in a recognition site of the restriction enzymes HindIII and NdeI of pETDuet-tmaLDH-tmaDp so as to produce a fusion protein His-tmaLDH::tmaDp expression vector pETDuet-tmaLDH::tmaDp (SEQ ID NO:65) wherein Hiswherein His-tmaLDH and tmaDp are bounded by a spacer sequence SEQ ID NO:142.

Then, the genomic DNA of Themotoga maritima (ATCC 43589) is used as a mold and following synthetic oligo DNA is used as a primersynthetic oligo DNAs SEQ ID NO:66 having a sequence recognized by NcoI and SEQ ID NO:62 having a sequence recognized by HindIII are used as primers for performing PCR, so as to obtain a DNA amplified product of 977 bp.

The DNA amplified product is cut by restriction enzymes NcoI and HindIII, and inserted in the same restriction enzyme site of pCDFDuet-1 (manufactured by Novagen) so as to produce a tmaLDH expression vector pCDFDuet-tmaLDH.

Then, the genomic DNA of Themotoga maritima (ATCC 43589) is used as a mold and following synthetic oligo DNA is used as a primersynthetic oligo DNAs SEQ ID NO:63 having a sequence recognized by NdeI and SEQ ID NO:64 having a sequence recognized by XhoI are used as primers for performing PCR, so as to obtain a DNA amplified product of 1371 bp.

The DNA amplified product is cut by restriction enzymes NdeI and XhoI, and inserted in the same restriction enzyme site of pCDFDuet-tmaLDH so as to produce a tmaLDH, tmaDp coexpression vector pCDFDuet-tmaLDH-tmaDp (SEQ ID NO:67).

E. coli BL21 (DE3) is transformed by the expression vectors pETDuet-tmaLDH::tmaDp and pCDFDuet-tmaLDH-tmaDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 37° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Thus obtained fusion protein derived from thermophile can be applied to a lactic acid sensor and the like.

### EXAMPLE 25

### Preparation of Fusion Protein (His-ppuMDH::goxDp) (SEQ ID NO:78 and 79) of Malic Dehydrogenase (ppuMDH) Derived from Pseudomonas putida and Diaphorase (goxDp) Derived from Gluconobacter oxydans

Genomic DNA is prepared from Pseudomonas putida (ATCC 47054) by a normal method. The genomic DNA is used as a mold and following synthetic oligo DNA is used as a primersynthetic oligo DNAs SEQ ID NO:73 having a sequence recognized by BamHI and SEQ ID NO:74 having a sequence recognized by HindIII are used as primers for performing PCR, so as to obtain a DNA amplified product of 1288 bp.

The DNA amplified product is cut by restriction enzymes BamHI and HindIII, and inserted in the same restriction enzyme site of pETDuet-1 (manufactured by Novagen) so as to produce a His-ppuMDH expression vector pETDuet-ppuMDH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:49 having a sequence recognized by NdeI and SEQ ID NO:50 having a sequence recognized by XhoI are used as primers for performing PCR, so as to obtain a DNA amplified product of 1428 bp.

The DNA amplified product is cut by restriction enzymes NdeI and XhoI, and inserted in the same restriction enzyme site of pETDuet-ppuMDH so as to produce a His-ppuMDH, goxDp coexpression vector pETDuet-ppuMDH-goxDp.

Then, following 5′ end phosphorylated synthetic oligo DNA is equivalent mixed in a TE buffer, and heated, and then annealed by gradual cooling.

SEQ ID NO:51

SEQ ID NO:52

The DNA fragment is inserted in a recognition site of the restriction enzymes HindIII and NdeI of pETDuet-ppuMDH-goxDp so as to produce a fusion protein His-ppuMDH::goxDp expression vector pETDuet-ppuMDH::goxDp (SEQ ID NO:75) wherein Hiswherein His-ppuMDH and goxDp are bounded by a spacer sequence SEQ ID NO:142.

Then, the genomic DNA of Pseudomonas putida (ATCC 47054) is used as a mold and following synthetic oligo DNA is used as a primersynthetic oligo DNAs SEQ ID NO:76 having a sequence recognized by NcoI and SEQ ID NO:74 having a sequence recognized by HindIII are used as primers for performing PCR, so as to obtain a DNA amplified product of 1286 bp.

The DNA amplified product is cut by restriction enzymes NcoI and HindIII, and inserted in the same restriction enzyme site of pCDFDuet-1 (manufactured by Novagen) so as to produce a ppuMDH expression vector pCDFDuet-ppuMDH.

Then, the genomic DNA of Gluconobacter oxydans (ATCC 621H) is used as a mold and following synthetic oligo DNA is used as a primersynthetic oligo DNAs SEQ ID NO:49 having a sequence recognized by NdeI and SEQ ID NO:50 having a sequence recognized by XhoI are used as primers for performing PCR, so as to obtain a DNA amplified product of 1428 bp.

The DNA amplified product is cut by restriction enzymes NdeI and XhoI, and inserted in the same restriction enzyme site of pCDFDuet-ppuMDH so as to produce a ppuMDH, goxDp coexpression vector pCDFDuet-ppuMDH-goxDp (SEQ ID NO:77)

E. coli BL21 (DE3) is transformed by the expression vectors pETDuet-ppuMDH::goxDp and pCDFDuet-ppuMDH-goxDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### COMPARISON EXAMPLE 25

### Preparation of ppuMDH and goxDp as a Control

Genomic DNA is prepared from Pseudomonas putida (ATCC 47054) by a normal method. The genomic DNA is used as a mold and following synthetic oligo DNA is used as a primersynthetic oligo DNAs SEQ ID NO:80 having a sequence recognized by NdeI and SEQ ID NO:81 having a sequence recognized by XhoI are used as primers for performing PCR, so as to obtain a DNA amplified product of about 1290 bp.

The DNA amplified product is cut by restriction enzymes NdeI and XhoI, and inserted in the same restriction enzyme site of pET-21a(+) (manufactured by Novagen) so as to produce a ppuMDH-His expression vector pET21-ppuMDH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:49 having a sequence recognized by NdeI and SEQ ID NO:60 having a sequence recognized by XhoI are used as primers for performing PCR, so as to obtain a DNA amplified product of about 1420 bp.

The DNA amplified product is cut by restriction enzymes NdeI and XhoI, and inserted in the same restriction enzyme site of pET-21a(+) (manufactured by Novagen) so as to produce a goxDp-His expression vector pET21-goxDp.

E. coli BL21 (DE3) is transformed according to a normal method by independently using the expression vectors pET21-ppuMDH and pET21-goxDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### EXAMPLE 26

### Malic Acid Sensor

Example 26 will be explained with reference to FIG. 28 and FIG. 8. The sensor in Example 26 is a malic acid sensor for determining the quantity of malic acid in a sample solution. The enzyme electrode part of the malic acid sensor in Example 26 will be explained with reference to FIG. 28.

The enzyme electrode part is constituted as follows. A conductive base plate 20 is glassy carbon with diameter of 3 mm. The fusion protein (His-ppuMDH::goxDp) prepared in Example 25, and ferrocene-bounded polyallylamine (Fc-PAA) are crosslinked and immobilized on the conductive base plate 20 by PDGDE. Each of the components is immobilized at following optimal concentrations, respectively. That is to say, the components are prepared at His-ppuMDH::goxDp (ppuMDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute a malic acid sensor. The counter electrode 5 is formed of a platinum wire, and the reference electrode 6 is made of a silver/silver chloride electrode. These electrodes measure current and the like via a potentiostat 10, respectively. 0.1 M PIPES-NaOH buffer aqueous solution (pH 7.5) containing a predetermined concentration of malic acid and 1 mM NAD is used for the sample solution 3. The measured temperature is held at 30° C. by a constant temperature circulation vessel.

The quantity is determined using the above-mentioned malic acid sensor as follows. A potential of 300 mV is applied to the reference electrode 6 in the reaction electrode 4. At that time, malic acid in the sample solution 3 is oxidized to pyruvic acid in the presence of malic dehydrogenase, and NAD is reduced to NADH by this reaction. Then, NADH is oxidized to NAD in the presence of diaphorase fused to the malic dehydrogenase. Ferrocene as an electron transfer mediator is oxidized by this reaction so as to produce a ferricinium ions. The ferricinium ions receive electrons from the reaction electrode 4 and are reduced to ferrocene (because the potential of 300 mV is applied to the reference electrode 6 in the reaction electrode 4). The current by movement of the electrons at the reaction electrode 4 is measured, so as to measure the concentration of malic acid in the sample solution.

### COMPARISON EXAMPLE 26

### Malic Acid Sensor

Comparison Example 26 will be explained with reference to FIG. 30 and FIG. 8. The sensor in Comparison Example 26 is a malic acid sensor for determining the quantity of malic acid in a sample solution. The enzyme electrode part of the malic acid sensor in Comparison Example 26 will be explained with reference to FIG. 30.

The enzyme electrode part is constituted as follows. A conductive base plate 20 is glassy carbon with diameter of 3 mm. Malic dehydrogenase (ppuMDH), diaphorase (goxDp), and ferrocene-bounded polyallylamine (Fc-PAA) are crosslinked and immobilized on the conductive base plate 20 by PEGDE. Malic dehydrogenase (ppuMDH) is malic dehydrogenase derived from Pseudomonas putida prepared in Comparison Example 25, and diaphorase (goxDp) is diaphorase derived from Gluconobacter oxydans. Each of the components is immobilized at following optimal concentrations, respectively. That is to say, the components are prepared at ppuMDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

In this enzyme electrode part, as is similar to Example 26, the concentration of malic acid in the sample solution is measured. The difference between the results of Example 26 and Comparison Example 26 is similar to the difference between the lines A and B of FIG. 11.

From Example 26 and Comparison Example 26, it becomes apparent that the malic acid sensor of Example 26 has higher sensitivity to the malic acid concentration and can determine the quantity of malic acid with lower concentration than the malic acid sensor of Comparison Example 26. Although the quantities of malic dehydrogenase and diaphorase immobilized on the enzyme electrodes are identical, malic dehydrogenase and diaphorase are fused in the enzyme electrode of Example 26, so that both of the enzymes are held physically near each other. Therefore, it is considered that both of the enzymes can receive and give electrons quickly via NAD/NADH.

### EXAMPLE 27

### Malic Acid Fuel Cell

Example 27 will be explained with reference to FIG. 28 and FIG. 9. The fuel cell in Example 27 is a malic acid fuel cell using malic acid as fuel. The anode electrode part of the malic acid fuel cell in Example 27 will be explained with reference to FIG. 28.

The anode electrode part is constituted as follows. A conductive base plate 20 is glassy carbon of 0.5 cm². The fusion protein (His-ppuMDH::goxDp) prepared in Example 25, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-ppuMDH::goxDp (ppuMDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 of the malic acid fuel cell in Example 27 is made of a platinum plate of 0.5 cm². Electrolyte solution 3 is 50 mM phosphate buffer (pH 7.5) containing 100 mM sodium chloride, 5 mM malic acid and 1 mM nicotinamide-adeninedinucleotide. A porous polypropylene film (thickness of 20 μm) 17 is inserted between the anode electrode 15 and the cathode electrode 16, and the electrodes are located in the electrolyte solution 3 (10 mL) in the water jacket cell 1 with the cover 2. The measured temperature is held at 30° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 1025 μA/cm²

Maximum power: 98 μW/cm²

### COMPARISON EXAMPLE 27

### Malic Acid Fuel Cell

Comparison Example 27 will be explained with reference to FIG. 30 and FIG. 9. The fuel cell in Comparison Example 27 is a malic acid fuel cell using malic acid as fuel. The anode electrode part of the malic acid fuel cell in Comparison Example 27 will be explained with reference to FIG. 30.

The anode electrode part is constituted as follows. A conductive base plate 20 is glassy carbon of 0.5 cm². Malic dehydrogenase (ppuMDH), diaphorase (goxDp), and ferrocene-bounded polyallylamine (Fc-PAA) are crosslinked and immobilized on the conductive base plate 20 by PEGDE. Malic dehydrogenase (ppuMDH) is malic dehydrogenase derived from Pseudomonas putida prepared in Comparison Example 25. Diaphorase (goxDp) is diaphorase derived from Gluconobacter oxydans. Each of the components is immobilized at following optimal concentrations, respectively. That is to say, the components are prepared at ppuMDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

As is similar to Example 27, the voltage-current characteristic is measured using the malic acid fuel cell of Comparison Example 27.

The output of the cell is observed as follows.

Short circuit current density: 297 μA/cm²

Maximum power: 30 μW/cm²

From Example 27 and Comparison Example 27, it becomes apparent that the malic acid fuel cell of Example 27 has higher current density and can pick up larger current that the malic acid fuel cell of Comparison Example 27. Although the quantities of malic dehydrogenase and diaphorase immobilized on the enzyme electrodes are identical, malic dehydrogenase and diaphorase are fused in the enzyme electrode of Example 27, so that both of the enzymes are held physically near each other. Therefore, it is considered that both of the enzymes can receive and give electrons quickly via NAD/NADH.

### EXAMPLE 28

### Malic Acid Electrochemical Reaction Device

Example 28 will be explained with reference to FIG. 28 and FIG. 8. The malic acid electrochemical reaction device in Example 28 is a malic acid electrochemical reaction device for producing pyruvic acid using malic acid as a substrate. The enzyme electrode part of the malic acid electrochemical reaction device of Example 28 will be explained with reference to FIG. 28.

The enzyme electrode part is constituted as follows. A conductive base plate 20 is glassy carbon of 0.5 cm². The fusion protein (His-ppuMDH::goxDp) prepared in Example 25, and ferrocene-bounded polyallylamine (Fc-PAA) are crosslinked and immobilized on the conductive base plate 20 by PEGDE. Each of the components is immobilized at following optimal concentrations, respectively. The components are prepared at His-ppuMDH::goxDp (ppuMDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode is used as the reaction electrode 4 of FIG. 8, so as to constitute a malic acid electrochemical reaction device. A 3-pole cell using a silver chloride electrode as the reference electrode 6 and a platinum wire as the counter electrode 5 is constituted. 50 mM phosphate buffer (pH 7.5) containing 100 mM sodium chloride, 5 mM malic acid and 1 mM nicotinamide-adeninedinucleotide is used as the sample solution 3. The temperature is held at 30° C. by a constant temperature circulation vessel, and the potential of 0.3 VvsAg/AgCl under a nitrogen atmosphere in the water jacket cell 1 is applied for 100 minutes, so that the quantity of the product is determined by high-speed liquid chromatography.

Pyruvic acid is detected from the reaction electrolyte. Therefore, it is recognized that a reaction charge volume and a produced pyruvic acid volume have high correlation, and the reaction advances quantitatively.

### COMPARISON EXAMPLE 28

### Malic Acid Electrochemical Reaction Device

Comparison Example 28 will be explained with reference to FIG. 30 and FIG. 8. The malic acid electrochemical reaction device in Comparison Example 28 is a malic acid electrochemical reaction device for producing pyruvic acid using malic acid as a substrate. The enzyme electrode part of the malic acid electrochemical reaction device of Comparison Example 28 will be explained with reference to FIG. 30.

The enzyme electrode part is constituted as follows. A conductive base plate 20 is glassy carbon of 0.5 cm². Malic dehydrogenase (ppuMDH), diaphorase (goxDp), and ferrocene-bounded polyallylamine (Fc-PAA) are crosslinked and immobilized on the conductive base plate 20 by PEGDE. Malic dehydrogenase (ppuMDH) is malic dehydrogenase derived from Pseudomonas putida prepared in Comparison Example 25, and diaphorase (goxDp) is a diaphorase derived from Gluconobacter oxydans. Each of the components is immobilized at following optimal concentrations, respectively. That is to say, the components are prepared at ppuMDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

Using this enzyme electrode, as is similar to Example 28, the quantity of the product is determined by high-speed chromatography.

Pyruvic acid is detected from the reaction electrolyte. Therefore, it is recognized that a reaction charge volume and a produced pyruvic acid volume have high correlation, and the reaction advances quantitatively. However, it is recognized that a reaction charge volume per unit time has a smaller value than that of Example 28.

From Example 28 and Comparison Example 28, it becomes apparent that the malic acid electrochemical reaction device of Example 28 has a higher reaction charge volume per unit time and can transform malic dehydrogenase into pyruvic acid more efficiently than the malic acid electrochemical reaction device of Comparison Example 28. Although the quantities of malic dehydrogenase and diaphorase immobilized on the enzyme electrodes are identical, malic dehydrogenase and diaphorase are fused in the enzyme electrode of Example 28, so that both of the enzymes are held physically near each other. Therefore, it is considered that both of the enzymes can receive and give electrons quickly via NAD/NADH.

### EXAMPLE 29

### Preparation of Fusion Protein (His-pfuMDH::tmaDp) (SEQ ID NO:87 and 88) of Malic Dehydrogenase (pfuMDH) Derived from Pyrococcus furiosus and Diaphorase (tmaDp) Derived from Themotoga maritima

Genomic DNA is prepared from Pyrococcus furiosus (ATCC 43587) by a normal method. The genomic DNA is used as a mold and following synthetic oligo DNA is used as a primersynthetic oligo DNAs SEQ ID NO:82 having a sequence recognized by BamHI and SEQ ID NO:83 having a sequence recognized by HindIII are used as primers for performing PCR, so as to obtain a DNA amplified product of 1327 bp.

The DNA amplified product is cut by restriction enzymes BamHI and HindIII, and inserted in the same restriction enzyme site of pETDuet-1 (manufactured by Novagen) so as to produce a His-pfuMDH expression vector pETDuet-pfuMDH.

Then, genomic DNA is prepared from Themotoga maritima (ATCC 43589) by a normal method. The genomic DNA is used as a mold and following synthetic oligo DNA is used as a primersynthetic oligo DNAs SEQ ID NO:63 having a sequence recognized by NdeI and SEQ ID NO:64 having a sequence recognized by XhoI are used as primers for performing PCR, so as to obtain a DNA amplified product of 1371 bp.

The DNA amplified product is cut by restriction enzymes NdeI and XhoI, and inserted in the same restriction enzyme site of pETDuet-pfuMDH so as to produce a His-pfuMDH, tmaDp coexpression vector pETDuet-pfuMDH-tmaDp.

Then, following 5′ end phosphorylated synthetic oligo DNA is equivalent mixed in a TE buffer, and heated, and then annealed by gradual cooling.

SEQ ID NO:51

SEQ ID NO:52

The DNA fragment is inserted in a recognition site of the restriction enzymes HindIII and NdeI of pETDuet-pfuMDH-tmaDp so as to produce a fusion protein His-pfuMDH::tmaDp expression vector pETDuet-pfuMDH::tmaDp (SEQ ID NO:84) wherein Hiswherein His-pfuMDH and tmaDp are bounded by a spacer sequence SEQ ID NO:142.

Then, the genomic DNA of Pyrococcus furiosus (ATCC 43587) is used as a mold and following synthetic oligo DNA is used as a primersynthetic oligo DNAs SEQ ID NO:85 having a sequence recognized by NcoI and SEQ ID NO:83 having a sequence recognized by HindIII are used as primers for performing PCR, so as to obtain a DNA amplified product of 1325 bp.

The DNA amplified product is cut by restriction enzymes NcoI and HindIII, and inserted in the same restriction enzyme site of pCDFDuet-1 (manufactured by Novagen) so as to produce a pfuMDH expression vector pCDFDuet-pfuMDH.

Then, the genomic DNA of Themotoga maritima (ATCC 43589) is used as a mold and following synthetic oligo DNA is used as a primersynthetic oligo DNAs SEQ ID NO:63 having a sequence recognized by NdeI and SEQ ID NO:64 having a sequence recognized by XhoI are used as primers for performing PCR, so as to obtain a DNA amplified product of 1371 bp.

The DNA amplified product is cut by restriction enzymes NdeI and XhoI, and inserted in the same restriction enzyme site of pCDFDuet-pfuMDH so as to produce a pfuMDH, tmaDp coexpression vector pCDFDuet-pfuMDH-tmaDp (SEQ ID NO:86).

E. coli BL21 (DE3) is transformed by the expression vectors pETDuet-pfuMDH::tmaDp and pCDFDuet-pfuMDH-tmaDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 37° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.) so as to remove solid foreign matters. After it is confirmed that a target expression protein exists in a supernatant by SDS-PAGE, the induction-expressed His-tag fusion protein is purified using a nickel chelate column.

By using thus obtained fusion protein derived from thermophile, a malic acid sensor, a malic fuel cell and the like can be constituted.

### EXAMPLE 33

### Preparation of Fusion Protein (His-bmaEDH::goxDp) (SEQ ID NO:96 and 97) of Glutamic Dehydrogenase (bmaEDH) Derived from Burkholderia mallei and Diaphorase (goxDp) Derived from Gluconobacter oxydans

Genomic DNA is prepared from Burkholderia mallei (ATCC 23344) by a normal method. The genomic DNA is used as a mold and following synthetic oligo DNA is used as a primersynthetic oligo DNAs SEQ ID NO:91 having a sequence recognized by BamHI and SEQ ID NO:92 having a sequence recognized by HindIII are used as primers for performing PCR, so as to obtain a DNA amplified product of 1324 bp.

The DNA amplified product is cut by restriction enzymes BamHI and HindIII, and inserted in the same restriction enzyme site of pETDuet-1 (manufactured by Novagen) so as to produce a His-bmaEDH expression vector pETDuet-bmaEDH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:49 having a sequence recognized by NdeI and SEQ ID NO:50 having a sequence recognized by XhoI are used as primers for performing PCR, so as to obtain a DNA amplified product of 1428 bp.

The DNA amplified product is cut by restriction enzymes NdeI and XhoI, and inserted in the same restriction enzyme site of pETDuet-bmaEDH so as to produce a His-bmaEDH, goxDp coexpression vector pETDuet-bmaEDH-goxDp.

Then, following 5′ end phosphorylated synthetic oligo DNA is equivalent mixed in a TE buffer, and heated, and then annealed by gradual cooling.

SEQ ID NO:51

SEQ ID NO:52

The DNA fragment is inserted in a recognition site of the restriction enzymes HindIII and NdeI of pETDuet-bmaEDH-goxDp so as to produce a fusion protein His-bmaEDH::goxDp expression vector pETDuet-bmaEDH::goxDp (SEQ ID NO:93) wherein Hiswherein His-bmaEDH and goxDp are bounded by a spacer sequence SEQ ID NO:142.

Then, the genomic DNA of Burkholderia mallei (ATCC 23344) is used as a mold and following synthetic oligo DNA is used as a primersynthetic oligo DNAs SEQ ID NO:94 having a sequence recognized by NcoI and SEQ ID NO:92 having a sequence recognized by HindIII are used as primers for performing PCR, so as to obtain a DNA amplified product of 1322 bp.

The DNA amplified product is cut by restriction enzymes NcoI and HindIII, and inserted in the same restriction enzyme site of pCDFDuet-1 (manufactured by Novagen) so as to produce a bmaEDH expression vector pCDFDuet-bmaEDH.

Then, the genomic DNA of Gluconobacter oxydans (ATCC 621H) is used as a mold and following synthetic oligo DNA is used as a primersynthetic oligo DNAs SEQ ID NO:49 having a sequence recognized by NdeI and SEQ ID NO:50 having a sequence recognized by XhoI are used as primers for performing PCR, so as to obtain a DNA amplified product of 1428 bp.

The DNA amplified product is cut by restriction enzymes NdeI and XhoI, and inserted in the same restriction enzyme site of pCDFDuet-bmaEDH so as to produce a bmaEDH, goxDp coexpression vector pCDFDuet-bmaEDH-goxDp (SEQ ID NO:95).

E. coli BL21 (DE3) is transformed by the expression vectors pETDuet-bmaEDH::goxDp and pCDFDuet-bmaEDH-goxDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### COMPARISON EXAMPLE 33

### Preparation of bmaEDH and goxDp as a Control

Genomic DNA is prepared from Burkholderia mallei (ATCC 23344) by a normal method. The genomic DNA is used as a mold and following synthetic oligo DNA is used as a primersynthetic oligo DNAs SEQ ID NO:98 having a sequence recognized by NdeI and SEQ ID NO:99 having a sequence recognized by XhoI are used as primers for performing PCR, so as to obtain a DNA amplified product of about 1320 bp.

The DNA amplified product is cut by restriction enzymes NdeI and XhoI, and inserted in the same restriction enzyme site of pET-21a(+) (manufactured by Novagen) so as to produce a bmaEDH-His expression vector pET21-bmaEDH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:49 having a sequence recognized by NdeI and SEQ ID NO:60 having a sequence recognized by XhoI are used as primers for performing PCR, so as to obtain a DNA amplified product of about 1420 bp.

The DNA amplified product is cut by restriction enzymes NdeI and XhoI, and inserted in the same restriction enzyme site of pET-21a(+) (manufactured by Novagen) so as to produce a goxDp-His expression vector pET21-goxDp.

E. coli BL21 (DE3) is transformed according to a normal method by independently using the expression vectors pET21-bmaEDH and pET21-goxDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### EXAMPLE 34

### Glutamic Acid Sensor

Example 34 will be explained with reference to FIG. 34 and FIG. 8. The sensor in Example 34 is a glutamic acid sensor for determining the quantity of glutamic acid in a sample solution. The enzyme electrode part of the glutamic acid sensor in Example 34 will be explained with reference to FIG. 34.

The enzyme electrode part is constituted as follows. A conductive base plate 20 is glassy carbon with diameter of 3 mm. The fusion protein (His-bmaEDH::goxDp) prepared in Example 33, and ferrocene-bounded polyallylamine (Fc-PAA) are crosslinked and immobilized on the conductive base plate 20 by PDGDE. Each of the components is immobilized at following optimal concentrations, respectively. That is to say, the components are prepared at His-bmaEDH::goxDp (bmaEDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute a glutamic acid sensor. The counter electrode 5 is formed of a platinum wire, and the reference electrode 6 is made of a silver/silver chloride electrode. These electrodes measure current and the like via a potentiostat 10, respectively. 0.1 M PIPES-NaOH buffer aqueous solution (pH 7.5) containing a predetermined concentration of glutamic acid and 1 mM NAD is used for the sample solution 3. The measured temperature is held at 30° C. by a constant temperature circulation vessel.

The quantity is determined using the above-mentioned glutamic acid sensor as follows. A potential of 300 mV is applied to the reference electrode 6 in the reaction electrode 4. At that time, glutamic acid in the sample solution 3 is oxidized to 2-oxoglutaric acid in the presence of glutamic dehydrogenase, and NAD is reduced to NADH by this reaction. Then, NADH is oxidized to NAD in the presence of diaphorase fused to the glutamic dehydrogenase. Ferrocene as an electron transfer mediator is oxidized by this reaction so as to produce a ferricinium ions. The ferricinium ions receive electrons from the reaction electrode 4 and are reduced to ferrocene (because the potential of 300 mV is applied to the reference electrode 6 in the reaction electrode 4). The current by movement of the electrons at the reaction electrode 4 is measured, so as to measure the concentration of glutamic acid in the sample solution.

### COMPARISON EXAMPLE 34

### Glutamic Acid Sensor

Comparison Example 34 will be explained with reference to FIG. 36 and FIG. 8. The sensor in Comparison Example 34 is a glutamic acid sensor for determining the quantity of glutamic acid in a sample solution. The enzyme electrode part of the glutamic acid sensor in Comparison Example 34 will be explained with reference to FIG. 36.

The enzyme electrode part is constituted as follows. A conductive base plate 20 is glassy carbon with diameter of 3 mm. Glutamic dehydrogenase (bmaEDH), diaphorase (goxDp), and ferrocene-bounded polyallylamine (Fc-PAA) are crosslinked and immobilized on the conductive base plate 20 by PEGDE. Glutamic dehydrogenase (bmaEDH) is glutamic dehydrogenase derived from Burkholderia mallei prepared in Comparison Example 33, and diaphorase (goxDp) is diaphorase derived from Gluconobacter oxydans. Each of the components is immobilized at following optimal concentrations, respectively. That is to say, the components are prepared at bmaEDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

In this enzyme electrode part, as is similar to Example 34, the concentration of glutamic acid in the sample solution is measured. The difference between the results of Example 34 and Comparison Example 34 is similar to the difference between the lines A and B of FIG. 11.

From Example 34 and Comparison Example 34, it becomes apparent that the glutamic acid sensor of Example 34 has higher sensitivity to the glutamic acid concentration and can determine the quantity of glutamic acid with lower concentration than the glutamic acid sensor of Comparison Example 34. Although the quantities of glutamic dehydrogenase and diaphorase immobilized on the enzyme electrodes are identical, glutamic dehydrogenase and diaphorase are fused in the enzyme electrode of Example 34, so that both of the enzymes are held physically near each other. Therefore, it is considered that both of the enzymes can receive and give electrons quickly via NAD/NADH.

### EXAMPLE 35

### Glutamic Acid Fuel Cell

Example 35 will be explained with reference to FIG. 34 and FIG. 9. The fuel cell in Example 35 is a glutamic acid fuel cell using glutamic acid as fuel. The anode electrode part of the glutamic acid fuel cell in Example 35 will be explained with reference to FIG. 34.

The anode electrode part is constituted as follows. A conductive base plate 20 is glassy carbon of 0.5 cm². The fusion protein (His-bmaEDH::goxDp) prepared in Example 33, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-bmaEDH::goxDp (bmaEDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 of the glutamic acid fuel cell in Example 35 is made of a platinum plate of 0.5 cm². Electrolyte solution 3 is 50 mM phosphate buffer (pH 7.5) containing 100 mM sodium chloride, 5 mM glutamic acid and 1 mM nicotinamide-adeninedinucleotide. A porous polypropylene film (thickness of 20 μm) 17 is inserted between the anode electrode 15 and the cathode electrode 16, and the electrodes are located in the electrolyte solution 3 (10 mL) in the water jacket cell 1 with the cover 2. The measured temperature is held at 30° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 1225 μA/cm²

Maximum power: 105 μW/cm²

### COMPARISON EXAMPLE 35

### Glutamic Acid Fuel Cell

Comparison Example 35 will be explained with reference to FIG. 36 and FIG. 9. The fuel cell in Comparison Example 35 is a glutamic acid fuel cell using glutamic acid as fuel. The anode electrode part of the glutamic acid fuel cell in Comparison Example 35 will be explained with reference to FIG. 36.

The anode electrode part is constituted as follows. A conductive base plate 20 is glassy carbon of 0.5 cm². Glutamic dehydrogenase (bmaEDH), diaphorase (goxDp), and ferrocene-bounded polyallylamine (Fc-PAA) are crosslinked and immobilized on the conductive base plate 20 by PEGDE. Glutamic dehydrogenase (bmaEDH) is glutamic dehydrogenase derived from Burkholderia mallei prepared in Comparison Example 33. Diaphorase (goxDp) is diaphorase derived from Gluconobacter oxydans. Each of the components is immobilized at following optimal concentrations, respectively. That is to say, the components are prepared at bmaEDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

As is similar to Example 35, the voltage-current characteristic is measured using the glutamic acid fuel cell of Comparison Example 35.

The output of the cell is observed as follows.

Short circuit current density: 386 μA/cm²

Maximum power: 33 μW/cm²

From Example 35 and Comparison Example 35, it becomes apparent that the glutamic acid fuel cell of Example 35 has higher current density and can pick up larger current that the glutamic acid fuel cell of Comparison Example 35. Although the quantities of glutamic dehydrogenase and diaphorase immobilized on the enzyme electrodes are identical, glutamic dehydrogenase and diaphorase are fused in the enzyme electrode of Example 35, so that both of the enzymes are held physically near each other. Therefore, it is considered that both of the enzymes can receive and give electrons quickly via NAD/NADH.

### EXAMPLE 36

### Glutamic Acid Electrochemical Reaction Device

Example 36 will be explained with reference to FIG. 34 and FIG. 8. The glutamic acid electrochemical reaction device in Example 36 is a glutamic acid electrochemical reaction device for producing 2-oxoglutaric acid using glutamic acid as a substrate. The enzyme electrode part of the glutamic acid electrochemical reaction device of Example 36 will be explained with reference to FIG. 34.

The enzyme electrode part is constituted as follows. A conductive base plate 20 is glassy carbon of 0.5 cm². The fusion protein (His-bmaEDH::goxDp) prepared in Example 33, and ferrocene-bounded polyallylamine (Fc-PAA) are crosslinked and immobilized on the conductive base plate 20 by PEGDE. Each of the components is immobilized at following optimal concentrations, respectively. The components are prepared at His-bmaEDH::goxDp (bmaEDH: 0.3 unit and goxDp: 0.6 unit), Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode is used as the reaction electrode 4 of FIG. 8, so as to constitute a glutamic acid electrochemical reaction device. A 3-pole cell using a silver chloride electrode as the reference electrode 6 and a platinum wire as the counter electrode 5 is constituted. 50 mM phosphate buffer (pH 7.5) containing 100 mM sodium chloride, 5 mM glutamic acid and 1 mM nicotinamide-adeninedinucleotide is used as the sample solution 3. The temperature is held at 30° C. by a constant temperature circulation vessel, and the potential of 0.3 VvsAg/AgCl under a nitrogen atmosphere in the water jacket cell 1 is applied for 100 minutes, so that the quantity of the product is determined by high-speed liquid chromatography.

2-oxoglutaric acid is detected from the reaction electrolyte. Therefore, it is recognized that a reaction charge volume and a produced 2-oxoglutaric acid volume have high correlation, and the reaction advances quantitatively.

### COMPARISON EXAMPLE 36

### Glutamic Acid Electrochemical Reaction Device

Comparison Example 36 will be explained with reference to FIG. 36 and FIG. 8. The glutamic acid electrochemical reaction device in Comparison Example 36 is a glutamic acid electrochemical reaction device for producing 2-oxoglutaric acid using glutamic acid as a substrate. The enzyme electrode part of the glutamic acid electrochemical reaction device of Comparison Example 36 will be explained with reference to FIG. 36.

The enzyme electrode part is constituted as follows. A conductive base plate 20 is glassy carbon of 0.5 cm². Glutamic dehydrogenase (bmaEDH), diaphorase (goxDp), and ferrocene-bounded polyallylamine (Fc-PAA) are crosslinked and immobilized on the conductive base plate 20 by PEGDE. Glutamic dehydrogenase (bmaEDH) is glutamic dehydrogenase derived from Burkholderia mallei prepared in Comparison Example 33, and diaphorase (goxDp) is diaphorase derived from Gluconobacter oxydans. Each of the components is immobilized at following optimal concentrations, respectively. That is to say, the components are prepared at bmaEDH: 0.3 unit, goxDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

Using this enzyme electrode, as is similar to Example 36, the quantity of the product is determined by high-speed chromatography.

2-oxoglutaric acid is detected from the reaction electrolyte. Therefore, it is recognized that a reaction charge volume and a produced 2-oxoglutaric acid volume have high correlation, and the reaction advances quantitatively. However, it is recognized that a reaction charge volume per unit time has a smaller value than that of Example 36.

From Example 36 and Comparison Example 36, it becomes apparent that the glutamic acid electrochemical reaction device of Example 36 has a higher reaction charge volume per unit time and can transform glutamic dehydrogenase into 2-oxoglutaric acid more efficiently than the glutamic acid electrochemical reaction device of Comparison Example 36. Although the quantities of glutamic dehydrogenase and diaphorase immobilized on the enzyme electrodes are identical, glutamic dehydrogenase and diaphorase are fused in the enzyme electrode of Example 36, so that both of the enzymes are held physically near each other. Therefore, it is considered that both of the enzymes can receive and give electrons quickly via NAD/NADH.

### EXAMPLE 37

### Preparation of Fusion Protein (His-pfuEDH::tmaDp) (SEQ ID NO:105 and 106) of Glutamic Dehydrogenase (pfuEDH) Derived from Pyrococcus furiosus and Diaphorase (tmaDp) Derived from Themotoga maritima

Genomic DNA is prepared from Pyrococcus furiosus (ATCC 43587) by a normal method. The genomic DNA is used as a mold and following synthetic oligo DNA is used as a primersynthetic oligo DNAs SEQ ID NO:100 having a sequence recognized by BamHI and SEQ ID NO:101 having a sequence recognized by HindIII are used as primers for performing PCR, so as to obtain a DNA amplified product of 1282 bp.

The DNA amplified product is cut by restriction enzymes BamHI and HindIII, and inserted in the same restriction enzyme site of pETDuet-1 (manufactured by Novagen) so as to produce a His-pfuEDH expression vector pETDuet-pfuEDH.

Then, genomic DNA is prepared from Themotoga maritima (ATCC 43589) by a normal method. The genomic DNA is used as a mold and following synthetic oligo DNA is used as a primersynthetic oligo DNAs SEQ ID NO:63 having a sequence recognized by NdeI and SEQ ID NO:64 having a sequence recognized by XhoI are used as primers for performing PCR, so as to obtain a DNA amplified product of 1371 bp.

The DNA amplified product is cut by restriction enzymes NdeI and XhoI, and inserted in the same restriction enzyme site of pETDuet-pfuEDH so as to produce a His-pfuEDH, tmaDp coexpression vector pETDuet-pfuEDH-tmaDp. Then, following 5′ end phosphorylated synthetic oligo DNA is equivalent mixed in a TE buffer, and heated, and then annealed by gradual cooling.

SEQ ID NO:51

SEQ ID NO:52

The DNA fragment is inserted in a recognition site of the restriction enzymes HindIII and NdeI of pETDuet-pfuEDH-tmaDp so as to produce a fusion protein His-pfuEDH::tmaDp expression vector pETDuet-pfuEDH::tmaDp (SEQ ID NO:102) wherein Hiswherein His-pfuEDH and tmaDp are bounded by a spacer sequence SEQ ID NO:142.

Then, the genomic DNA of Pyrococcus furiosus (ATCC 43587) is used as a mold and following synthetic oligo DNA is used as a primersynthetic oligo DNAs SEQ ID NO:103 having a sequence recognized by NcoI and SEQ ID NO:101 having a sequence recognized by HindIII are used as primers for performing PCR, so as to obtain a DNA amplified product of 1280 bp.

The DNA amplified product is cut by restriction enzymes NcoI and HindIII, and inserted in the same restriction enzyme site of pCDFDuet-1 (manufactured by Novagen) so as to produce a pfuEDH expression vector pCDFDuet-pfuEDH.

Then, the genomic DNA of Themotoga maritima (ATCC 43589) is used as a mold and following synthetic oligo DNA is used as a primersynthetic oligo DNAs SEQ ID NO:63 having a sequence recognized by NdeI and SEQ ID NO:64 having a sequence recognized by XhoI are used as primers for performing PCR, so as to obtain a DNA amplified product of 1371 bp.

The DNA amplified product is cut by restriction enzymes NdeI and XhoI, and inserted in the same restriction enzyme site of pCDFDuet-pfuEDH so as to produce a pfuEDH, tmaDp coexpression vector pCDFDuet-pfuEDH-tmaDp (SEQ ID NO:104).

E. coli BL21 (DE3) is transformed by the expression vectors pETDuet-pfuEDH::tmaDp and pCDFDuet-pfuEDH-tmaDp according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 37° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

By using thus obtained fusion protein derived from thermophile, a glutamic acid sensor and the like can be constituted.

### EXAMPLE 41

### Preparation of Fusion Protein (His-sceADH::goxALDH) (SEQ ID NO:113 and 114) of Alcohol Dehydrogenase (sceADH) Derived from Saccharomyces cerevisiae and Aldehyde Dehydrogenase (goxALDH) Derived from Gluconobacter oxydans

Genomic DNA is prepared from Saccharomyces cerevisiae (ATCC 47058) by a normal method. The genomic DNA is used as a mold and following synthetic oligo DNA is used as a primersynthetic oligo DNAs SEQ ID NO:27 having a sequence recognized by BamHI and SEQ ID NO:28 having a sequence recognized by HindIII are used as primers for performing PCR, so as to obtain a DNA amplified product of 1075 bp.

The DNA amplified product is cut by restriction enzymes BamHI and HindIII, and inserted in the same restriction enzyme site of pETDuet-1 (manufactured by Novagen) so as to produce a His-sceADH expression vector pETDuet-sceADH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:109 having a sequence recognized by NdeI and SEQ ID NO:110 having a sequence recognized by XhoI are used as primers for performing PCR, so as to obtain a DNA amplified product of 1560 bp.

The DNA amplified product is cut by restriction enzymes NdeI and XhoI, and inserted in the same restriction enzyme site of pETDuet-sceADH so as to produce a His-sceADH, goxALDH coexpression vector pETDuet-sceADH-goxALDH.

Then, following 5′ end phosphorylated synthetic oligo DNA is equivalent mixed in a TE buffer, and heated, and then annealed by gradual cooling.

SEQ ID NO:29

SEQ ID NO:30

The DNA fragment is inserted in a recognition site of the restriction enzymes HindIII and NdeI of pETDuet-sceADH-goxALDH so as to produce a fusion protein His-sceADH::goxALDH expression vector pETDuet-sceADH::goxALDH (SEQ ID NO:111) wherein Hiswherein His-sceADH and goxALDH are bounded by a spacer sequence SEQ ID NO:146.

Then, the genomic DNA of Saccharomyces cerevisiae (ATCC 47058) is used as a mold and following synthetic oligo DNA is used as a primersynthetic oligo DNAs SEQ ID NO:32 having a sequence recognized by NcoI and SEQ ID NO:28 having a sequence recognized by HindIII are used as primers for performing PCR, so as to obtain a DNA amplified product of 1073 bp.

The DNA amplified product is cut by restriction enzymes NcoI and HindIII, and inserted in the same restriction enzyme site of pCDFDuet-1 (manufactured by Novagen) so as to produce a sceADH expression vector pCDFDuet-sceADH.

Then, the genomic DNA of Gluconobacter oxydans (ATCC 621H) is used as a mold and following synthetic oligo DNA is used as a primersynthetic oligo DNAs SEQ ID NO:109 having a sequence recognized by NdeI and SEQ ID NO:110 having a sequence recognized by XhoI are used as primers for performing PCR, so as to obtain a DNA amplified product of 1560 bp.

The DNA amplified product is cut by restriction enzymes NdeI and XhoI, and inserted in the same restriction enzyme site of pCDFDuet-sceADH so as to produce a sceADH, goxALDH coexpression vector pCDFDuet-sceADH-goxALDH (SEQ ID NO:112).

E. coli BL21 (DE3) is transformed by the expression vectors pETDuet-sceADH::goxALDH and pCDFDuet-sceADH-goxALDH according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Then, genomic DNA is prepared from Pseudomonas putida KT2440 (ATCC 47054) by a normal method. The genomic DNA is used as a mold and following synthetic oligo DNA is used as a primersynthetic oligo DNAs SEQ ID NO:3 having a sequence recognized by NdeI and SEQ ID NO:14 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pET-21a(+) (manufactured by Novagen) so as to produce a ppuDp-His expression vector pET21-ppuDp.

E. coli BL21 (DE3) is transformed according to a normal method by independently using the expression vector pET21-ppuDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion proteins are purified using a nickel chelate column.

Thus obtained fusion protein can be applied to an alcohol sensor and the like.

### COMPARISON EXAMPLE 41

### Preparation of sceADH and goxALDH as a Control

Genomic DNA is prepared from Saccharomyces cerevisiae (ATCC 47058) by a normal method. The genomic DNA is used as a mold and following synthetic oligo DNA is used as a primersynthetic oligo DNAs SEQ ID NO:36 having a sequence recognized by NdeI and SEQ ID NO:37 having a sequence recognized by XhoI are used as primers for performing PCR, so as to obtain a DNA amplified product of about 1074 bp.

The DNA amplified product is cut by restriction enzymes NdeI and XhoI, and inserted in the same restriction enzyme site of pET-21a(+) (manufactured by Novagen) so as to produce a sceADH-His expression vector pET21-sceADH.

Then, genomic DNA is prepared from Gluconobacter oxydans (ATCC 621H) by a normal method. The genomic DNA is used as a mold and following synthetic oligo DNA is used as a primersynthetic oligo DNAs SEQ ID NO:109 having a sequence recognized by NdeI and SEQ ID NO:115 having a sequence recognized by XhoI are used as primers for performing PCR, so as to obtain a DNA amplified product of about 1557 bp.

The DNA amplified product is cut by restriction enzymes NdeI and XhoI, and inserted in the same restriction enzyme site of pET-21a(+) (manufactured by Novagen) so as to produce a goxALDH-His expression vector pET21-goxALDH.

Then, genomic DNA is prepared from Pseudomonas putida KT2440 (ATCC 47054) by a normal method. The genomic DNA is used as a mold and following synthetic oligo DNA is used as a primersynthetic oligo DNAs SEQ ID NO:3 having a sequence recognized by NdeI and SEQ ID NO:14 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pET-21a(+) (manufactured by Novagen) so as to produce a ppuDp-His expression vector pET21-ppuDp.

E. coli BL21 (DE3) is transformed according to a normal method by independently using the expression vectors pET21-sceADH, pET21-goxALDH and pET21-ppuDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### EXAMPLE 42

### Alcohol Sensor

Example 42 will be explained with reference to FIG. 40 and FIG. 8. The sensor in Example 42 is an alcohol sensor for determining the quantity of alcohol in a sample solution. The enzyme electrode part of the alcohol sensor in Example 42 will be explained with reference to FIG. 40.

The enzyme electrode part is constituted as follows. A conductive base plate 20 is glassy carbon with diameter of 3 mm. The fusion protein (His-sceADH::goxALDH) prepared in Example 41, diaphorase (ppuDp) derived from Pseudomonas putida and ferrocene-bounded polyallylamine (Fc-PAA) are crosslinked and immobilized on the conductive base plate 20 by PDGDE. Each of the components is immobilized at following optimal concentrations, respectively. That is to say, the components are prepared at His-sceADH::goxALDH (sceADH: 0.3 unit and goxALDH: 0.6 unit), ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute an alcohol sensor. The counter electrode 5 is formed of a platinum wire, and the reference electrode 6 is made of a silver/silver chloride electrode. These electrodes measure current and the like via a potentiostat 10, respectively. 0.1 M PIPES-NaOH buffer aqueous solution (pH 7.5) containing a predetermined concentration of alcohol (ethanol) and 1 mM NAD is used for the sample solution 3. The measured temperature is held at 37° C. by a constant temperature circulation vessel.

The quantity is determined using the above-mentioned alcohol sensor as follows. A potential of 300 mV is applied to the reference electrode 6 in the reaction electrode 4. At that time, alcohol in the sample solution 3 is oxidized to acetaldehyde in the presence of alcohol dehydrogenase, and NAD is reduced to NADH by this reaction. Then, acetaldehyde is oxidized to carboxylic acid, and at the same time, NAD is reduced to NADH. NADH produced by both of the enzyme reactions of alcohol dehydrogenase and aldehyde dehydrogenase is oxidized to NAD in the presence of aldehyde dehydrogenase fused to the alcohol dehydrogenase. Ferrocene as an electron transfer mediator is oxidized by this reaction so as to produce a ferricinium ions. The ferricinium ions receive electrons from the reaction electrode 4 and are reduced to ferrocene (because the potential of 300 mV is applied to the reference electrode 6 in the reaction electrode 4). The current by movement of the electrons at the reaction electrode 4 is measured, so as to measure the concentration of alcohol in the sample solution.

### COMPARISON EXAMPLE 42

### Alcohol Sensor

Comparison Example 42 will be explained with reference to FIG. 42 and FIG. 8. The sensor in Comparison Example 42 is a alcohol sensor for determining the quantity of alcohol in a sample solution. The enzyme electrode part of the alcohol sensor in Comparison Example 42 will be explained with reference to FIG. 42.

The enzyme electrode part is constituted as follows. A conductive base plate 20 is glassy carbon with diameter of 3 mm. Alcohol dehydrogenase (sceADH), aldehyde dehydrogenase (goxALDH), diaphorase (ppuDp) and ferrocene-bounded polyallylamine (Fc-PAA) are crosslinked and immobilized on the conductive base plate 20 by PEGDE. Alcohol dehydrogenase (sceADH) is alcohol dehydrogenase derived from Saccharomyces cerevisiae prepared in Comparison Example 41, and aldehyde dehydrogenase (goxALDH) is aldehyde dehydrogenase derived from Gluconobacter oxydans. Diaphorase (ppuDp) is diaphorase derived from Pseudomonas putida. Each of the components is immobilized at following optimal concentrations, respectively. That is to say, the components are prepared at sceADH: 0.3 unit, goxALDH: 0.6 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

In this enzyme electrode part, as is similar to Example 42, the concentration of alcohol in the sample solution is measured. The difference between the results of Example 42 and Comparison Example 42 is similar to the difference between the lines A and B of FIG. 11.

From Example 42 and Comparison Example 42, it becomes apparent that the alcohol sensor of Example 42 has higher sensitivity to the alcohol concentration and can determine the quantity of alcohol with lower concentration than the alcohol sensor of Comparison Example 42. Although the quantities of alcohol dehydrogenase, aldehyde dehydrogenase, and diaphorase immobilized on the enzyme electrodes are identical, alcohol dehydrogenase and aldehyde dehydrogenase are fused in the enzyme electrode of Example 42, so that both of the enzymes are held physically near each other. Therefore, it is considered that the volume of NADH produce in a unit time is large.

### EXAMPLE 43

### Alcohol Fuel Cell

Example 43 will be explained with reference to FIG. 40 and FIG. 9. The fuel cell in Example 43 is an alcohol fuel cell using alcohol as fuel. The anode electrode part of the alcohol fuel cell in Example 43 will be explained with reference to FIG. 40.

The anode electrode part is constituted as follows. A conductive base plate 20 is glassy carbon of 0.5 cm². The fusion protein (His-sceADH::goxALDH) prepared in Example 41, and diaphorase (ppuDp) derived from Pseudomonas putida, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-sceADH::goxALDH (sceADH: 0.3 unit and goxALDH: 0.6 unit), ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 of the alcohol fuel cell in Example 43 is made of a platinum plate of 0.5 cm². Electrolyte solution 3 is 50 mM phosphate buffer (pH 7.5) containing 100 mM sodium chloride, 5 mM alcohol (ethanol) and 1 mM nicotinamide-adeninedinucleotide. A porous polypropylene film (thickness of 20 μm) 17 is inserted between the anode electrode 15 and the cathode electrode 16, and the electrodes are located in the electrolyte solution 3 (10 mL) in the water jacket cell 1 with the cover 2. The measured temperature is held at 37° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 1620 μA/cm²

Maximum power: 172 μW/cm²

### COMPARISON EXAMPLE 43

### Alcohol Fuel Cell

Comparison Example 43 will be explained with reference to FIG. 42 and FIG. 9. The fuel cell in Comparison Example 43 is an alcohol fuel cell using alcohol as fuel. The anode electrode part of the alcohol fuel cell in Comparison Example 43 will be explained with reference to FIG. 42.

The anode electrode part is constituted as follows. A conductive base plate 20 is glassy carbon of 0.5 cm². Alcohol dehydrogenase (sceADH), aldehyde dehydrogenase (goxALDH), diaphorase (ppuDp), and ferrocene-bounded polyallylamine (Fc-PAA) are crosslinked and immobilized on the conductive base plate 20 by PEGDE. Alcohol dehydrogenase (sceADH) is alcohol dehydrogenase derived from Saccharomyces cerevisiae prepared in Comparison Example 41. Aldehyde dehydrogenase (goxALDH) is aldehyde dehydrogenase derived from Gluconobacter oxydans. Diaphorase (ppuDp) is diaphorase derived from Pseudomonas putida. Each of the components is immobilized at following optimal concentrations, respectively. That is to say, the components are prepared at sceADH: 0.3 unit, goxALDH: 0.6 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

As is similar to Example 43, the voltage-current characteristic is measured using the alcohol fuel cell of Comparison Example 43.

The output of the cell is observed as follows.

Short circuit current density: 502 μA/cm²

Maximum power: 50 μW/cm²

From Example 43 and Comparison Example 43, it becomes apparent that the alcohol fuel cell of Example 43 has higher current density and can pick up larger current that the alcohol fuel cell of Comparison Example 43. Although the quantities of alcohol dehydrogenase, aldehyde dehydrogenase and diaphorase immobilized on the enzyme electrodes are identical, alcohol dehydrogenase and aldehyde dehydrogenase are fused in the enzyme electrode of Example 43, so that both of the enzymes are held physically near each other. Therefore, it is considered that the volume of NADH produce in a unit time is large.

### EXAMPLE 44

### Alcohol Electrochemical Reaction Device

Example 44 will be explained with reference to FIG. 40 and FIG. 8. The alcohol electrochemical reaction device in Example 44 is an alcohol electrochemical reaction device for producing carboxylic acid (acetic acid) using alcohol as a substrate. The enzyme electrode part of the alcohol electrochemical reaction device of Example 44 will be explained with reference to FIG. 40.

The enzyme electrode part is constituted as follows. A conductive base plate 20 is glassy carbon of 0.5 cm². The fusion protein (His-sceADH::goxALDH) prepared in Example 41, diaphorase (ppuDp) derived from Pseudomonas putida, and ferrocene-bounded polyallylamine (Fc-PAA) are crosslinked and immobilized on the conductive base plate 20 by PEGDE. Each of the components is immobilized at following optimal concentrations, respectively. The components are prepared at His-sceADH::goxALDH (sceADH: 0.3 unit and goxALDH: 0.6 unit), ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode is used as the reaction electrode 4 of FIG. 8, so as to constitute an alcohol electrochemical reaction device. A 3-pole cell using a silver chloride electrode as the reference electrode 6 and a platinum wire as the counter electrode 5 is constituted. 50 mM phosphate buffer (pH 7.5) containing 100 mM sodium chloride, 5 mM alcohol and 1 mM nicotinamide-adeninedinucleotide is used as the sample solution 3. The temperature is held at 37° C. by a constant temperature circulation vessel, and the potential of 0.3 VvsAg/AgCl under a nitrogen atmosphere in the water jacket cell 1 is applied for 100 minutes, so that the quantity of the product is determined by high-speed liquid chromatography.

Carboxylic acid (acetic acid) is detected from the reaction electrolyte. Therefore, it is recognized that a reaction charge volume and a produced carboxylic acid (acetic acid) volume have high correlation, and the reaction advances quantitatively.

### COMPARISON EXAMPLE 44

### Alcohol Electrochemical Reaction Device

Comparison Example 44 will be explained with reference to FIG. 42 and FIG. 8. The alcohol electrochemical reaction device in Comparison Example 44 is an alcohol electrochemical reaction device for producing carboxylic acid (acetic acid) using alcohol as a substrate. The enzyme electrode part of the alcohol electrochemical reaction device of Comparison Example 44 will be explained with reference to FIG. 42.

The enzyme electrode part is constituted as follows. A conductive base plate 20 is glassy carbon of 0.5 cm². Alcohol dehydrogenase (sceADH), aldehyde dehydrogenase (goxALDH), diaphorase (ppuDp), and ferrocene-bounded polyallylamine (Fc-PAA) are crosslinked and immobilized on the conductive base plate 20 by PEGDE. Alcohol dehydrogenase (sceADH) is alcohol dehydrogenase derived from Saccharomyces cerevisiae prepared in Comparison Example 41, and aldehyde dehydrogenase (goxALDH) is aldehyde dehydrogenase derived from Gluconobacter oxydans. Diaphorase (ppuDp) is diaphorase derived from Pseudomonas putida. Each of the components is immobilized at following optimal concentrations, respectively. That is to say, the components are prepared at sceADH: 0.3 unit, goxALDH: 0.6 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

Using this enzyme electrode, as is similar to Example 44, the quantity of the product is determined by high-speed chromatography.

Carboxylic acid (acetic acid) is detected from the reaction electrolyte. Therefore, it is recognized that a reaction charge volume and a produced carboxylic acid (acetic acid) volume have high correlation, and the reaction advances quantitatively. However, it is recognized that a reaction charge volume per unit time has a smaller value than that of Example 44. From Example 44 and Comparison Example 44, it becomes apparent that the alcohol electrochemical reaction device of Example 44 has a higher reaction charge volume per unit time and can transform alcohol dehydrogenase into 2-oxoglutaric acid more efficiently than the alcohol electrochemical reaction device of Comparison Example 44. Although the quantities of alcohol dehydrogenase, aldehyde dehydrogenase and diaphorse immobilized on the enzyme electrodes are identical, alcohol dehydrogenase and aldehyde dehydrogenase are fused in the enzyme electrode of Example 44, so that both of the enzymes are held physically near each other. Therefore, it is considered that the volume of NADH produce in a unit time is large.

### EXAMPLE 45

### Preparation of Fusion Protein (His-pfuADH::tthALDH) (SEQ ID NO:120 and 121) of Alcohol Dehydrogenase (pfuADH) Derived from Pyrococcus furiosus and Aldehyde Dehydrogenase (tthALDH) Derived from Thermus thermophilus

Genomic DNA is prepared from Pyrococcus furiosus (ATCC 43587) by a normal method. The genomic DNA is used as a mold and following synthetic oligo DNA is used as a primersynthetic oligo DNAs SEQ ID NO:38 having a sequence recognized by BamHI and SEQ ID NO:39 having a sequence recognized by HindIII are used as primers for performing PCR, so as to obtain a DNA amplified product of 1147 bp.

The DNA amplified product is cut by restriction enzymes BamHI and HindIII, and inserted in the same restriction enzyme site of pETDuet-1 (manufactured by Novagen) so as to produce a His-pfuADH expression vector pETDuet-pfuADH.

Then, genomic DNA is prepared from Thermus thermophilus (ATCC BAA-163) by a normal method. The genomic DNA is used as a mold and following synthetic oligo DNA is used as a primersynthetic oligo DNAs SEQ ID NO:116 having a sequence recognized by NdeI and SEQ ID NO:117 having a sequence recognized by XhoI are used as primers for performing PCR, so as to obtain a DNA amplified product of 1614 bp.

The DNA amplified product is cut by restriction enzymes NdeI and XhoI, and inserted in the same restriction enzyme site of pETDuet-pfuADH so as to produce a His-pfuADH, tthALDH coexpression vector pETDuet-pfuADH-tthALDH.

Then, following 5′ end phosphorylated synthetic oligo DNA is equivalent mixed in a TE buffer, and heated, and then annealed by gradual cooling.

SEQ ID NO:29

SEQ ID NO:30

The DNA fragment is inserted in a recognition site of the restriction enzymes HindIII and NdeI of pETDuet-pfuADH-tthALDH so as to produce a fusion protein His-pfuADH::tthALDH expression vector pETDuet-pfuADH::tthALDH (SEQ ID NO:118) wherein Hiswherein His-pfuADH and tthALDH are bounded by a spacer sequence SEQ ID NO:146.

Then, the genomic DNA of Pyrococcus furiosus (ATCC 43587) is used as a mold and following synthetic oligo DNA is used as a primersynthetic oligo DNAs SEQ ID NO:41 having a sequence recognized by NcoI and SEQ ID NO:39 having a sequence recognized by HindIII are used as primers for performing PCR, so as to obtain a DNA amplified product of 1145 bp.

The DNA amplified product is cut by restriction enzymes NcoI and HindIII, and inserted in the same restriction enzyme site of pCDFDuet-1 (manufactured by Novagen) so as to produce a pfuADH expression vector pCDFDuet-pfuADH.

Then, the genomic DNA of Thermus thermophilus (ATCC BAA-163) is used as a mold and following synthetic oligo DNA is used as a primersynthetic oligo DNAs SEQ ID NO:116 having a sequence recognized by NdeI and SEQ ID NO:117 having a sequence recognized by XhoI are used as primers for performing PCR, so as to obtain a DNA amplified product of 1614 bp.

The DNA amplified product is cut by restriction enzymes NdeI and XhoI, and inserted in the same restriction enzyme site of pCDFDuet-pfuADH so as to produce a pfuADH, tthALDH coexpression vector pCDFDuet-pfuADH-tthALDH (SEQ ID NO:119).

E. coli BL21 (DE3) is transformed by the expression vectors pETDuet-pfuADH::tthALDH and pCDFDuet-pfuADH-tthALDH according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Then, genomic DNA is prepared from Pyrococcus horikoshii KT2440 (ATCC 700860) by a normal method. The genomic DNA is used as a mold and following synthetic oligo DNA is used as a primersynthetic oligo DNAs SEQ ID NO:17 having a sequence recognized by NdeI and SEQ ID NO:26 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pET-21a(+) (manufactured by Novagen) so as to produce a phoDp-His expression vector pET21-phoDp.

E. coli BL21 (DE3) is transformed by the expression vector pET21-phoDp according to a normal method. The transformant can be selected as a resistant strain against antibiotics such as ampicillin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Thus obtained fusion protein can be applied to an alcohol sensor and the like.

### EXAMPLE 49

### Preparation of Fusion Protein (His-busGDH::ecoISO) (SEQ ID NO:127 and 128) of Glucose Dehydrogenase (busGDH) Derived from Bacillus subtilis and Xyloseisomerase (ecoISO) Derived from Escherichia coli

Genomic DNA is prepared from Bacillus subtilis (ATCC 27370) by a normal method. The genomic DNA is used as a mold and following synthetic oligo DNA is used as a primersynthetic oligo DNAs SEQ ID NO:1 having a sequence recognized by BamHI and SEQ ID NO:2 having a sequence recognized by HindIII are used as primers for performing PCR, so as to obtain a DNA amplified product of 805 bp.

The DNA amplified product is cut by restriction enzymes BamHI and HindIII, and inserted in the same restriction enzyme site of pETDuet-1 (manufactured by Novagen) so as to produce a His-busGDH expression vector pETDuet-busGDH.

Then, genomic DNA is prepared from Escherichia coli (ATCC 29425) by a normal method. The genomic DNA is used as a mold and following synthetic oligo DNA is used as a primersynthetic oligo DNAs SEQ ID NO:123 having a sequence recognized by NdeI and SEQ ID NO:124 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pETDuet-busGDH so as to produce a His-busGDH, ecoISO coexpression vector pETDuet-busGDH-ecoISO.

Then, following 5′ end phosphorylated synthetic oligo DNA is equivalent mixed in a TE buffer, and heated, and then annealed by gradual cooling.

SEQ ID NO:29

SEQ ID NO:30

The DNA fragment is inserted in a recognition site of the restriction enzymes HindIII and NdeI of pETDuet-busGDH-ecoISO so as to produce a fusion protein His-busGDH::ecoISO expression vector pETDuet-busGDH::ecoISO (SEQ ID NO:125) wherein Hiswherein His-busGDH and ecoISO are bounded by a spacer sequence SEQ ID NO:146.

Then, the genomic DNA of Bacillus subtilis (ATCC 27370) is used as a mold and following synthetic oligo DNA is used as a primersynthetic oligo DNAs SEQ ID NO:8 having a sequence recognized by NcoI and SEQ ID NO:2 having a sequence recognized by HindIII are used as primers for performing PCR, so as to obtain a DNA amplified product of 805 bp.

The DNA amplified product is cut by restriction enzymes NcoI and HindIII, and inserted in the same restriction enzyme site of pCDFDuet-1 (manufactured by Novagen) so as to produce a busGDH expression vector pCDFDuet-busGDH.

Then, the genomic DNA of Escherichia coli (ATCC 29425) is used as a mold and following synthetic oligo DNA is used as a primersynthetic oligo DNAs SEQ ID NO:123 having a sequence recognized by NdeI and SEQ ID NO:124 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pCDFDuet-busGDH so as to produce a busGDH, ecoISO coexpression vector pCDFDuet-busGDH-ecoISO (SEQ ID NO:126).

E. coli BL21 (DE3) is transformed by the expression vectors pETDuet-busGDH::ecoISO and pCDFDuet-busGDH-ecoISO according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Then, genomic DNA is prepared from Pseudomonas putida KT2440 (ATCC-47054) by a normal method. The genomic DNA is used as a mold and following synthetic oligo DNA is used as a primersynthetic oligo DNAs SEQ ID NO:3 having a sequence recognized by NdeI and SEQ ID NO:14 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pET-21a(+) (manufactured by Novagen) so as to produce a ppuDp-His expression vector pET21-ppuDp.

E. coli BL21 (DE3) is transformed according to a normal method by independently using the expression vector pET21-ppuDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion proteins are purified using a nickel chelate column.

### COMPARISON EXAMPLE 49

### Preparation of busGDH and ecoISO as a Control

Genomic DNA is prepared from Bacillus subtilis (ATCC 27370) by a normal method. The genomic DNA is used as a mold and following synthetic oligo DNA is used as a primersynthetic oligo DNAs SEQ ID NO:12 having a sequence recognized by NdeI and SEQ ID NO:13 having a sequence recognized by XhoI are used as primers for performing PCR, so as to obtain a DNA amplified product of about 800 bp.

The DNA amplified product is cut by restriction enzymes NdeI and XhoI, and inserted in the same restriction enzyme site of pET-21a(+) (manufactured by Novagen) so as to produce a busGDH-His expression vector pET21-busGDH.

Then, genomic DNA is prepared from Escherichia coli (ATCC 29425) by a normal method. The genomic DNA is used as a mold and following synthetic oligo DNA is used as a primersynthetic oligo DNAs SEQ ID NO:123 having a sequence recognized by NdeI and SEQ ID NO:129 having a sequence recognized by XhoI are used as primers for performing PCR, so as to obtain a DNA amplified product of about 1341 bp.

The DNA amplified product is cut by restriction enzymes NdeI and XhoI, and inserted in the same restriction enzyme site of pET-21a(+) (manufactured by Novagen) so as to produce a ecoISO-His expression vector pET21-ecoISO.

Then, genomic DNA is prepared from Pseudomonas putida KT2440 (ATCC 47054) by a normal method. The genomic DNA is used as a mold and following synthetic oligo DNA is used as a primersynthetic oligo DNAs SEQ ID NO:3 having a sequence recognized by NdeI and SEQ ID NO:14 having a sequence recognized by XhoI are used as primers for performing PCR, so as to obtain a DNA amplified product of 729 bp.

The DNA amplified product is cut by restriction enzymes NdeI and XhoI, and inserted in the same restriction enzyme site of pET-21a(+) (manufactured by Novagen) so as to produce a ppuDp-His expression vector pET21-ppuDp.

E. coli BL21 (DE3) is transformed according to a normal method by independently using the expression vectors pET21-busGDH, pET21-ecoISO and pET21-ppuDp. The transformant can be selected as a resistant strain against antibiotics such as ampicillin.

The resultant transformant is pre-cultured for one night in 10 mL of a LB culture medium, to which antibiotics such as ampicillin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

### EXAMPLE 50

### Fructose Sensor

Example 50 will be explained with reference to FIG. 46 and FIG. 8. The sensor in Example 50 is a fructose sensor for determining the quantity of fructose in a sample solution. The enzyme electrode part of the fructose sensor in Example 50 will be explained with reference to FIG. 46.

The enzyme electrode part is constituted as follows. A conductive base plate 20 is glassy carbon with diameter of 3 mm. The fusion protein (His-busGDH::ecoISO) prepared in Example 49, diaphorase (ppuDp) and ferrocene-bounded polyallylamine (Fc-PAA) are crosslinked and immobilized on the conductive base plate 20 by PDGDE. Diaphorase (ppuDp) is diaphorase derived from Pseudomonas putida. Each of the components is immobilized at following optimal concentrations, respectively. That is to say, the components are prepared at His-busGDH::ecoISO (busGDH: 0.3 unit and ecoISO: 0.6 unit), ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode part is used as a reaction electrode 4 in FIG. 8, so as to constitute a fructose: sensor. The counter electrode 5 is formed of a platinum wire, and the reference electrode 6 is made of a silver/silver chloride electrode. These electrodes measure current and the like via a potentiostat 10, respectively. 0.1 M PIPES-NaOH buffer aqueous solution (pH 7.5) containing a predetermined concentration of fructose and 1 mM NAD is used for the sample solution 3. The measured temperature is held at 37° C. by a constant temperature circulation vessel.

The quantity is determined using the above-mentioned fructose sensor as follows. A potential of 300 mV is applied to the reference electrode 6 in the reaction electrode 4. At that time, fructose in the sample solution 3 is oxidized to glucose in the presence of xyloseisomerase. Then, glucose is oxidized to gluconolactone, and at the same time, NAD is reduced to NADH. The produced NADH is oxidized to NAD in the presence of diaphorase. Ferrocene as an electron transfer mediator is oxidized by this reaction so as to produce a ferricinium ions. The ferricinium ions receive electrons from the reaction electrode 4 and are reduced to ferrocene (because the potential of 300 mV is applied to the reference electrode 6 in the reaction electrode 4). The current by movement of the electrons at the reaction electrode 4 is measured, so as to measure the concentration of fructose in the sample solution.

### COMPARISON EXAMPLE 50

### Fructose Sensor

Comparison Example 50 will be explained with reference to FIG. 48 and FIG. 8. The sensor in Comparison Example 50 is a fructose sensor for determining the quantity of fructose in a sample solution. The enzyme electrode part of the fructose sensor in Comparison Example 50 will be explained with reference to FIG. 48.

The enzyme electrode part is constituted as follows. A conductive base plate 20 is glassy carbon with diameter of 3 mm. Glucose dehydrogenase (busGDH), xyloseisomerase (ecoISO), diaphorase (ppuDp) and ferrocene-bounded polyallylamine (Fc-PAA) are crosslinked and immobilized on the conductive base plate 20 by PEGDE. Glucose dehydrogenase (busGDH) is glucose dehydrogenase derived from Bacillus subtilis prepared in Comparison Example 49, and xyloseisomerase (ecoISO) is xyloseisomerase derived from Escherichia coli. Diaphorase (ppuDp) is diaphorase derived from Pseudomonas putida. Each of the components is immobilized at following optimal concentrations, respectively. That is to say, the components are prepared at busGDH: 0.3 unit, ecoISO: 0.6 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

As is similar to Example 50, the concentration of fructose in the sample solution is measured using this enzyme electrode part. The difference between the results of Example 50 and Comparison Example 50 is similar to the difference between the lines A and B of FIG. 11.

From Example 50 and Comparison Example 50, it becomes apparent that the fructose sensor of Example 50 has higher sensitivity to the fructose concentration and can determine the quantity of fructose with lower concentration than the fructose sensor of Comparison Example 50. Although the quantities of glucose dehydrogenase, xyloseisomerase, and diaphorase immobilized on the enzyme electrodes are identical, glucose dehydrogenase and xyloseisomerase are fused in the enzyme electrode of Example 50, so that both of the enzymes are held physically near each other. Therefore, it is considered that fructose is quickly transformed and oxidized to gluconolactone via glucose, as a result of which the volume of NADH produced in a unit time is large.

### EXAMPLE 51

### Fructose Fuel Cell

Example 51 will be explained with reference to FIG. 46 and FIG. 9. The fuel cell in Example 51 is a fructose fuel cell using fructose as fuel. The anode electrode part of the fructose fuel cell in Example 51 will be explained with reference to FIG. 46.

The anode electrode part is constituted as follows. A conductive base plate 20 is glassy carbon of 0.5 cm². The fusion protein (His-busGDH::ecoISO) prepared in Example 49, and diaphorase (ppuDp) derived from Pseudomonas putida, and ferrocene-bounded polyallylamine (Fc-PAA) are crosslinked and immobilized on the conductive base plate 20 by PEGDE. Each of the components is immobilized at following optimal concentration ratios, respectively. The components are prepared at His-busGDH::ecoISO (busGDH: 0.3 unit and ecoISO: 0.6 unit), ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

A cathode electrode 16 of the fructose fuel cell in Example 51 is made of a platinum plate of 0.5 cm².

Electrolyte solution 3 is 50 mM phosphate buffer (pH 7.5) containing 100 mM sodium chloride, 5 mM fructose and 1 mM nicotinamide-adeninedinucleotide. A porous polypropylene film (thickness of 20 μm) 17 is inserted between the anode electrode 15 and the cathode electrode 16, and the electrodes are located in the electrolyte solution 3 (10 mL) in the water jacket cell 1 with the cover 2. The measured temperature is held at 37° C. by a constant temperature circulation vessel. The respective leads are connected with a potentiostat (Tohogiken, Model 2000) 10, and the voltage is changed from −1.2 V to 0.1 V, so as to measure a voltage-current characteristic.

The output of the cell is observed as follows.

Short circuit current density: 1105 μA/cm²

Maximum power: 100 μW/cm²

### COMPARISON EXAMPLE 51

### Fructose Fuel Cell

Comparison Example 51 will be explained with reference to FIG. 48 and FIG. 9. The fuel cell in Comparison Example 51 is a fructose fuel cell using fructose as fuel. The anode electrode part of the fructose fuel cell in Comparison Example 51 will be explained with reference to FIG. 48.

The anode electrode part is constituted as follows. A conductive base plate 20 is glassy carbon of 0.5 cm². Glucose dehydrogenase (busGDH), xyloseisomerase (ecoISO), diaphorase (ppuDp), and ferrocene-bounded polyallylamine (Fc-PAA) are crosslinked and immobilized on the conductive base plate 20 by PEGDE. Glucose dehydrogenase (busGDH) is glucose dehydrogenase derived from Bacillus subtilis prepared in Comparison Example 49. Xyloseisomerase (ecoISO) is xyloseisomerase derived from Escherichia coli. Diaphorase (ppuDp) is diaphorase derived from Pseudomonas putida. Each of the components is immobilized on the conductive base plate 20 at following optimal concentrations, respectively. That is to say, the components are prepared at busGDH: 0.3 unit, ecoISO: 0.6 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

As is similar to Example 51, the voltage-current characteristic is measured using the fructose fuel cell of Comparison Example 51.

The output of the cell is observed as follows.

Short circuit current density: 309 μA/cm²

Maximum power: 30 μW/cm²

From Example 51 and Comparison Example 51, it becomes apparent that the fructose fuel cell of Example 51 has higher current density and can pick up larger current that the fructose fuel cell of Comparison Example 51. Although the quantities of glucose dehydrogenase, xyloseisomerase and diaphorase immobilized on the enzyme electrodes are identical, glucose dehydrogenase and xyloseisomerase are fused in the enzyme electrode of Example 51, so that both of the enzymes are held physically near each other. Therefore, it is considered that fructose is quickly transformed and oxidized to gluconolactone via glucose, as a result of which the volume of NADH produced in a unit time is large.

### EXAMPLE 52

### Fructose Electrochemical Reaction Device

Example 52 will be explained with reference to FIG. 46 and FIG. 8. The fructose electrochemical reaction device in Example 52 is a fructose electrochemical reaction device for producing gluconolactone using fructose as a substrate. The enzyme electrode part of the fructose electrochemical reaction device of Example 52 will be explained with reference to FIG. 46.

The enzyme electrode part is constituted as follows. A conductive base plate 20 is glassy carbon of 0.5 cm². The fusion protein (His-busGDH::ecoISO) prepared in Example 49, diaphorase (ppuDp) derived from Pseudomonas putida, and ferrocene-bounded polyallylamine (Fc-PAA) are crosslinked and immobilized on the conductive base plate 20 by PEGDE. Each of the components is immobilized at following optimal concentrations, respectively. The components are prepared at His-busGDH::ecoISO (busGDH: 0.3 unit and ecoISO: 0.6 unit), ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

The enzyme electrode is used as the reaction electrode 4 of FIG. 8, so as to constitute a fructose electrochemical reaction device. A 3-pole cell using a silver chloride electrode as the reference electrode 6 and a platinum wire as the counter electrode 5 is constituted. 50 mM phosphate buffer (pH 7.5) containing 100 mM sodium chloride, 5 mM fructose and 1 mM nicotinamide-adeninedinucleotide is used as the sample solution 3. The temperature is held at 37° C. by a constant temperature circulation vessel, and the potential of 0.3 VvsAg/AgCl under a nitrogen atmosphere in the water jacket cell 1 is applied for 100 minutes, so that the quantity of the product is determined by high-speed liquid chromatography. Gluconolactone is detected from the reaction electrolyte. Therefore, it is recognized that a reaction charge volume and a produced gluconolactone volume have high correlation, and the reaction advances quantitatively.

### COMPARISON EXAMPLE 52

### Fructose Electrochemical Reaction Device

Comparison Example 52 will be explained with reference to FIG. 48 and FIG. 8. The fructose electrochemical reaction device in Comparison Example 52 is a fructose electrochemical reaction device for producing gluconolactone using fructose as a substrate. The enzyme electrode part of the fructose electrochemical reaction device of Comparison Example 52 will be explained with reference to FIG. 48.

The enzyme electrode part is constituted as follows. A conductive base plate 20 is glassy carbon of 0.5 cm². Glucose dehydrogenase (busGDH), xyloseisomerase (ecoISO), diaphorase (ppuDp), and ferrocene-bounded polyallylamine (Fc-PAA) are crosslinked and immobilized on the conductive base plate 20 by PEGDE. Glucose dehydrogenase (busGDH) is glucose dehydrogenase derived from Bacillus subtilis prepared in Comparison Example 49, and xyloseisomerase (ecoISO) is xyloseisomerase derived from Escherichia coli. Diaphorase (ppuDp) is diaphorase derived from Pseudomonas putida. Each of the components is immobilized at following optimal concentrations, respectively. That is to say, the components are prepared at busGDH: 0.3 unit, ecoISO: 0.6 unit, ppuDp: 0.6 unit, Fc-PAA: 16 μg, and PEGDE: 10 μg. The enzyme electrode part is manufactured by mixing each of aqueous solutions of the above-mentioned components on the electrode, leaving to stand in a room temperature for 2 or more hours, and drying them.

Using this enzyme electrode, as is similar to Example 52, the quantity of the product is determined by high-speed chromatography.

Gluconolactone is detected from the reaction electrolyte. Therefore, it is recognized that a reaction charge volume and a produced gluconolactone volume have high correlation, and the reaction advances quantitatively. However, it is recognized that a reaction charge volume per unit time has a smaller value than that of Example 52. From Example 52 and Comparison Example 52, it becomes apparent that the fructose electrochemical reaction device of Example 52 has a higher reaction charge volume per unit time and can transform fluctose into gluconolactone more efficiently than the fructose electrochemical reaction device of Comparison Example 52. Although the quantities of glucose dehydrogenase, xyloseisomerase and diaphorse immobilized on the enzyme electrodes are identical, glucose dehydrogenase and xyloseisomerase are fused in the enzyme electrode of Example 52, so that both of the enzymes are held physically near each other. Therefore, it is considered that fructose is quickly transformed and oxidized to gluconolactone via glucose, as a result of which the volume of NADH produced in a unit time is large.

### EXAMPLE 53

### Preparation of Fusion Protein (His-pfuGDH::tmaISO) (SEQ ID NO:134 and 135) of Glucose Dehydrogenase (pfuGDH) Derived from Pyrococcus furiosus and Xyloseisomerase (tmaISO) Derived from Thermotoga maritima

Genomic DNA is prepared from Pyrococcus furiosus (ATCC 43587) by a normal method. The genomic DNA is used as a mold and following synthetic oligo DNA is used as a primersynthetic oligo DNAs SEQ ID NO:15 having a sequence recognized by BamHI and SEQ ID NO:16 having a sequence recognized by HindIII are used as primers for performing PCR, so as to obtain a DNA amplified product of 799 bp.

The DNA amplified product is cut by restriction enzymes BamHI and HindIII, and inserted in the same restriction enzyme site of pETDuet-1 (manufactured by Novagen) so as to produce a His-pfuGDH expression vector pETDuet-pfuGDH.

Then, genomic DNA is prepared from Thermotoga maritima (ATCC 43589) by a normal method. The genomic DNA is used as a mold and following synthetic oligo DNA is used as a primersynthetic oligo DNAs SEQ ID NO:130 having a sequence recognized by NdeI and SEQ ID NO:131 having a sequence recognized by XhoI are used as primers for performing PCR, so as to obtain a DNA amplified product of 1356 bp.

The DNA amplified product is cut by restriction enzymes NdeI and XhoI, and inserted in the same restriction enzyme site of pETDuet-pfuGDH so as to produce a His-pfuGDH, tmaISO coexpression vector pETDuet-pfuGDH-tmaISO.

Then, following 5′ end phosphorylated synthetic oligo DNA is equivalent mixed in a TE buffer, and heated, and then annealed by gradual cooling.

SEQ ID NO:29

SEQ ID NO:30

The DNA fragment is inserted in a recognition site of the restriction enzymes HindIII and NdeI of pETDuet-pfuGDH-tmaISO so as to produce a fusion protein. His-pfuGDH::tmaISO expression vector pETDuet-pfuGDH::tmaISO (SEQ ID NO:132) wherein His-pfuGDH and tmaISO are bounded by a spacer sequence SEQ ID NO:146.

Then, the genomic DNA of Pyrococcus furiosus (ATCC 43587) is used as a mold and following synthetic oligo DNA is used as a primersynthetic oligo DNAs SEQ ID NO:20 having a sequence recognized by NcoI and SEQ ID NO:16 having a sequence recognized by HindIII are used as primers for performing PCR, so as to obtain a DNA amplified product of 797 bp.

The DNA amplified product is cut by restriction enzymes NcoI and HindIII, and inserted in the same restriction enzyme site of pCDFDuet-1 (manufactured by Novagen) so as to produce a pfuGDH expression vector pCDFDuet-pfuGDH.

Then, the genomic DNA of Thermotoga maritima (ATCC 43589) is used as a mold and following synthetic oligo DNA is used as a primersynthetic oligo DNAs SEQ ID N0130: having a sequence recognized by NdeI and SEQ ID NO:131 having a sequence recognized by XhoI are used as primers for performing PCR, so as to obtain a DNA amplified product of 1356 bp.

The DNA amplified product is cut by restriction enzymes NdeI and XhoI, and inserted in the same restriction enzyme site of pCDFDuet-pfuGDH so as to produce a pfuGDH, tmaISO coexpression vector pCDFDuet-pfuGDH-tmaISO (SEQ ID NO:133).

E. coli BL21 (DE3) is transformed by the expression vectors pETDuet-pfuGDH::tmaISO and pCDFDuet-pfuGDH-tmaISO according to a normal method. The transformant can be selected as a resistant strain against antibiotics such as ampicillin and streptomycin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Then, genomic DNA is prepared from Pyrococcus horikoshii KT2440 (ATCC 700860) by a normal method. The genomic DNA is used as a mold and following synthetic oligo DNA is used as a primersynthetic oligo DNAs SEQ ID NO:17 having a sequence recognized by NdeI and SEQ ID NO:26 having a sequence recognized by XhoI are used as primers for performing PCR, so as to obtain a DNA amplified product of 1344 bp.

The DNA amplified product is cut by restriction enzymes NdeI and XhoI, and inserted in the same restriction enzyme site of pET-21a(+) (manufactured by Novagen) so as to produce a phoDp=His expression vector pET21-phoDp.

E. coli BL21 (DE3) is transformed by the expression vector pET21-phoDp according to a normal method. The transformant can be selected as a resistant strain against antibiotics such as ampicillin.

The resultant transformant is pre-cultured for one night in 10 mL of a LB culture medium, to which antibiotics such as ampicillin and streptomycin are added. Then, 0.2 mL of the medium is added to 100 mL of an LB-Amp culture medium, and concussed and cultured for 4 hours at 30° C. and 170 rpm. Thereafter, IPTG is added (final concentration; 1 mM), and continues to be cultured at 37° C. for 4 to 12 hours. The IPTG induced transformant is collected (8000×g, 2 minutes, 4° C.), and resuspended in one-tenth volume of 4° C. PBS. The strains are fragmentized by freeze-thawing and sonication, and centrifuged (8000×g, 10 minutes, 4° C.), so as to remove solid foreign matters. After it is confirmed that a target expression protein exists in a supernatant by SDS-PAGE, the induction-expressed His-tag fusion protein is purified using a nickel chelate column.

Thus obtained fusion protein derived from thermophile can be applied to a fructose sensor and a fructose fuel cell.

By using thus obtained fusion protein derived from thermophile, a fructose sensor and the like can be constituted as the above-mentioned examples.

Though the present invention has been explained using a plurality of examples and comparison examples above, a sensor or a fuel cell using the fusion protein derived from thermophile has less deterioration of outputs over time, and excellent durability.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2005-356930, filed Dec. 9, 2005, which is hereby incorporated by reference herein in its entirety.

What is claimed is:

1. An enzyme electrode having a conductive base plate, and an enzyme electrically connected with the conductive base plate, wherein the enzyme is comprised of a fusion protein of a first enzyme to catalyze a chemical reaction for producing a first reaction product from a first reaction substrate and a second enzyme to catalyze a chemical reaction for producing a second reaction product from a second substrate, and at least one part of the first reaction product is identical to at least one part of the second reaction substrate.

2. The enzyme electrode described in claim 1, wherein the enzyme immobilized to the conductive base plate together with an electron transfer mediator.

3. The enzyme electrode described in claim 1, wherein the first enzyme is dehydrogenase, and the second enzyme is diaphorase.

4. The enzyme electrode described in claim 3, wherein the dehydrogenase is selected from glucose dehydrogenase, alcohol dehydrogenase, aldehyde dehydrogenase, lactic dehydrogenase, malic dehydrogenase and glutamic dehydrogenase.

5. The enzyme electrode described in claim 1, having diaphorase, wherein the first enzyme is alcohol dehydrogenase, and the second enzyme is aldehyde dehydrogenase.

6. The enzyme electrode described in claim 1, having diaphorase, wherein the first enzyme is isomerase, and the second enzyme is glucosededehydrogenase.

7. The enzyme electrode described in claim 1, having a glucose dehydrogenase activity and a diaphorase activity, wherein the fusion protein of dehydrogenase and diaphorase has (a) an amino acid sequence expressed by SEQ ID NO:11 or (b) an amino acid sequence expressed by SEQ ID NO:11, wherein modifications such as addition, deletion and substitution of one or more amino acids are implemented independently or in combination of a plurality of modifications within a range not to deteriorate each of the activities.

8. The enzyme electrode described in claim 1, having a glucose dehydrogenase activity and a diaphorase activity, wherein the fusion protein of dehydrogenase and diaphorase has (a) an amino acid sequence expressed by SEQ ID NO:23 or (b) an amino acid sequence expressed by SEQ ID NO:23, wherein modifications such as addition, deletion and substitution of one or more amino acids are implemented independently or in combination of a plurality of modifications within a range not to deteriorate each of the activities.

9. The enzyme electrode described in claim 1, having an alcohol dehydrogenase activity and a diaphorase activity, wherein the fusion protein of dehydrogenase and diaphorase has (a) an amino acid sequence expressed by SEQ ID NO:35 or (b) an amino acid sequence expressed by SEQ ID NO:35, wherein modifications such as addition, deletion and substitution of one or more amino acids are implemented independently or in combination of a plurality of modifications within a range not to deteriorate each of the activities.

10. The enzyme electrode described in claim 1, having an alcohol dehydrogenase activity and a diaphorase activity, wherein the fusion protein of dehydrogenase and diaphorase has (a) an amino acid sequence expressed by SEQ ID NO:44 or (b) an amino acid sequence expressed by SEQ ID NO:44, wherein modifications such as addition, deletion and substitution of one or more amino acids are implemented independently or in combination of a plurality of modifications within a range not to deteriorate each of the activities.

11. The enzyme electrode described in claim 1, having a lactic dehydrogenase activity and a diaphorase activity, wherein the fusion protein of dehydrogenase and diaphorase has (a) an amino acid sequence expressed by SEQ ID NO:57 or (b) an amino acid sequence expressed by SEQ ID NO:57, wherein modifications such as addition, deletion and substitution of one or more amino acids are implemented independently or in combination of a plurality of modifications within a range not to deteriorate each of the activities.

12. The enzyme electrode described in claim 1, having a lactic dehydrogenase activity and a diaphorase activity, wherein the fusion protein of dehydrogenase and diaphorase has (a) an amino acid sequence expressed by SEQ ID NO:69 or (b) an amino acid sequence expressed by SEQ ID NO:69, wherein modifications such as addition, deletion and substitution of one or more amino acids are implemented independently or in combination of a plurality of modifications within a range not to deteriorate each of the activities.

13. The enzyme electrode described in claim 1, having a malic dehydrogenase activity and a diaphorase activity, wherein the fusion protein of dehydrogenase and diaphorase has (a) an amino acid sequence expressed by SEQ ID NO:79 or (b) an amino acid sequence expressed by SEQ ID NO:79, wherein modifications such as addition, deletion and substitution of one or more amino acids are implemented independently or in combination of a plurality of modifications within a range not to deteriorate each of the activities.

14. The enzyme electrode described in claim 1, having a malic dehydrogenase activity and a diaphorase activity, wherein the fusion protein of dehydrogenase and diaphorase has (a) an amino acid sequence expressed by SEQ ID NO:88 or (b) an amino acid sequence expressed by SEQ ID NO:88, wherein modifications such as addition, deletion and substitution of one or more amino acids are implemented independently or in combination of a plurality of modifications within a range not to deteriorate each of the activities.

15. The enzyme electrode described in claim 1, having a glutamic dehydrogenase activity and a diaphorase activity, wherein the fusion protein of dehydrogenase and diaphorase has (a) an amino acid sequence expressed by SEQ ID NO:97 or (b) an amino acid sequence expressed by SEQ ID NO:97, wherein modifications such as addition, deletion and substitution of one or more amino acids are implemented independently or in combination of a plurality of modifications within a range not to deteriorate each of the activities.

16. The enzyme electrode described in claim 1, having a glutamic dehydrogenase activity and a diaphorase activity, wherein the fusion protein of dehydrogenase and diaphorase has (a) an amino acid sequence expressed by SEQ ID NO:106 or (b) an amino acid sequence expressed by SEQ ID NO:106, wherein modifications such as addition, deletion and substitution of one or more amino acids are implemented independently or in combination of a plurality of modifications within a range not to deteriorate each of the activities.

17. The enzyme electrode described in claim 5, having an alcohol dehydrogenase activity and an aldehyde dehydrogenase activity, wherein the fusion protein of alcohol dehydrogenase and aldehyde dehydrogenase has (a) an amino acid sequence expressed by SEQ ID NO:114 or (b) an amino acid sequence expressed by SEQ ID NO:114, wherein modifications such as addition, deletion and substitution of one or more amino acids are implemented independently or in combination of a plurality of modifications within a range not to deteriorate each of the activities.

18. The enzyme electrode described in claim 5, having an alcohol dehydrogenase activity and an aldehyde dehydrogenase activity, wherein the fusion protein of alcohol dehydrogenase and aldehyde dehydrogenase has (a) an amino acid sequence expressed by SEQ ID NO:121 or (b) an amino acid sequence expressed by SEQ ID NO:121, wherein modifications such as addition, deletion and substitution of one or more amino acids are implemented independently or in combination of a plurality of modifications within a range not to deteriorate each of the activities.

19. The enzyme electrode described in claim 6, having an isomerase activity and a glucose dehydrogenase activity, wherein the fusion protein of isomerase and glucose dehydrogenase has (a) an amino acid sequence expressed by SEQ ID NO:128 or (b) an amino acid sequence expressed by SEQ ID NO:128, wherein modifications such as addition, deletion and substitution of one or more amino acids are implemented independently or in combination of a plurality of modifications within a range not to deteriorate each of the activities.

20. The enzyme electrode described in claim 6, having an isomerase activity and a glucose dehydrogenase activity, wherein the fusion protein of isomerase and glucose dehydrogenase has (a) an amino acid sequence expressed by SEQ ID NO:135 or (b) an amino acid sequence expressed by SEQ ID NO:135, wherein modifications such as addition, deletion and substitution of one or more amino acids are implemented independently or in combination of a plurality of modifications within a range not to deteriorate each of the activities.

21. A sensor, wherein the enzyme electrode described in claim 1 is used as a detection portion for detecting a substance.

22. A fuel cell, wherein the enzyme electrode described in claim 1 is used as an anode.

23. An electrochemical reaction device, wherein the enzyme electrode described in claim 1 is used as a reaction electrode.
